(19) United States
(12) Patent Application Publication
(10) Pub. No.: US 2026/0340831 A1
(43) Pub. Date: Oct. 29, 2026

(54) COMPUTER-IMPLEMENTED HIGH THROUGHPUT SPECTRAL METHOD USEFUL FOR EVALUATION OF SEED TREATMENT PROTOCOLS

(71) Applicant: CORTEVA AGRISCIENCE LLC, INDIANAPOLIS, IN (US)

(72) Inventors: JEFFREY M. HEGSTAD, ANKENY, IA (US); ERIC SCHULTZ, CLIVE, IA (US); KAREN J. THOMPSON, URBANDALE, IA (US)

(73) Assignee: CORTEVA AGRISCIENCE LLC, INDIANAPOLIS, IN (US)

(21) Appl. No.: 19/116,914

(22) PCT Filed: Sep. 28, 2022
(86) PCT No.: PCT/US2022/077143

§ 371 Date: Mar. 28, 2025

(51) Int. Cl. G06T 7/00; A01C 1/00; G01N 21/31; G01N 33/00; G06T 7/90

(52) U.S. Cl. CPC G06T 7/0016; A01C 1/00; G01N 21/3151; G01N 33/0098; G06T 7/90; G01N 2021/3155; G06T 2207/10024; G06T 2207/10048; G06T 2207/30188

(57) ABSTRACT

Disclosed herein is a computer-implemented, high throughput total reflectance spectral analysis method for evaluating the efficacy of a seed treatment protocol in a greenhouse setting, comprising subjecting a plurality of seeds to a seed treatment protocol; allowing the plurality of seeds to germinate into plants; measuring the plant response to the seed treatment protocol at pre-determined growth stages using a high throughput total reflectance spectral analysis method; and determining the efficacy of the seed treatment protocol based upon the plant response.

### BACKGROUND

Disclosed herein are various high throughput analytical methods that are used to evaluate a plant treatment protocol. Specifically, the methods disclosed herein are used to measure plant responses to a seed treatment protocol where the plant is grown from a seed treated with a particular seed treatment protocol.

Cultivation of crops requires the efficient management of pests that can be harmful to the crops. It is believed that about 40% of global crop harvest is lost due to the pest damage each year. Significant efforts are directed to controlling or preventing the pest damage. Current methods include treating the plants with insecticides, fungicides, nematicides and other pesticide treatments that can control or eliminate harmful pathogens and pests. New and efficient treatment protocols are constantly being developed.

Treatment of the seeds prior to the germination and growing the plants from the treated seeds has been emerged as a powerful and effective technique to control the pests and pathogens. As the new treatments emerge and as pathogens develop resistance to the existing treatments, it is necessary to explore the various treatment protocols and their efficacy. Such explorations are ideally carried out in a greenhouse setting where the variables can be controlled, and where the tests can be conducted throughout the year.

The data gathered during such tests must be precise and reliable. Such tests also include gathering large amounts of data. The data must also be collected in a time-efficient manner. Some data collection methods that can be used in field settings have emerged; for example, data collection methods using Unmanned Arial Vehicles (UAV) combined with advanced photographic techniques have found increased use in the recent times. However, these techniques often are costly, and cannot be adapted to the tests conducted in a greenhouse setting. Accordingly, a high throughput method that can gather and analyze large amounts of data in a time efficient manner at a greenhouse setting needs to be developed.

### SUMMARY

Disclosed herein are a computer-implemented high throughput total reflectance spectral methods for evaluating the efficacy of a seed treatment protocol in a greenhouse setting. The methods may include subjecting a plurality of seeds to a seed treatment protocol and allowing the seeds to germinate into plants. The plant response to the seed treatment protocol may then be measured at a pre-determined time after the emergence using a high throughput total reflectance spectral method using a spectral range of 250 nm-2500 nm and a specific spectral interpolation of 1.0-4.0 nm and the efficacy of the seed treatment protocol may be determined based on the plant response. Further aspects, forms, objects, features, advantages, aspects, and benefits shall become apparent from the following description and drawings.

### BRIEF DESCRIPTION OF THE DRAWINGS

FIG. 1: Pixel area effect size by days after germination for corn hybrid P9998CYXR treated with different fungicide seed treatments compared to wild-type (not treated)

FIG. 1a: Pixel area effect size by days after germination for corn hybrid P9998CYXR treated with different fungicide seed treatments compared to wild-type (not treated) from 5 to 9 days after emergence.

FIG. 1b: Pixel area effect size by days after germination for corn hybrid P9998CYXR treated with different fungicide+polymer seed treatments compared to wild-type (not treated) from 5 to 9 days after emergence.

FIG. 2: Average standard deviation difference in hyperspectral wavelength (400-1000 nm) by days after germination for corn hybrid P998CYXR treated with different fungicide seed treatments compared to wild-type (not treated seed) from 5 to 9 days after emergence.

FIG. 2a: Average standard deviation difference in hyperspectral wavelength (400-1000 nm) by days after germination for corn hybrid P998CYXR treated with different fungicide seed treatments compared to wild-type (not treated seed) at 5 days after emergence.

FIG. 2b: Average standard deviation difference in hyperspectral wavelength (400-1000 nm) by days after germination for corn hybrid P998CYXR treated with different fungicide seed treatments compared to wild-type (not treated seed) at 7 days after emergence.

FIG. 2c: Average standard deviation difference in hyperspectral wavelength (400-1000 nm) by days after germination for corn hybrid P998CYXR treated with different fungicide seed treatments compared to wild-type (not treated seed) at 9 days after emergence.

FIG. 3: Average standard deviation difference in hyperspectral wavelength (400-1000 nm) by days after germination for corn hybrid P998CYXR treated with different fungicide+polymer seed treatments compared to wild-type (not treated seed) from 5 to 9 days after emergence.

FIG. 3a: Average standard deviation difference in hyperspectral wavelength (400-1000 nm) by days after germination for corn hybrid P998CYXR treated with different fungicide+polymer seed treatments compared to wild-type (not treated seed) at 5 days after emergence.

FIG. 3b: Average standard deviation difference in hyperspectral wavelength (400-1000 nm) by days after germination for corn hybrid P998CYXR treated with different fungicide+polymer seed treatments compared to wild-type (not treated seed) at 7 days after emergence.

FIG. 3c: Average standard deviation difference in hyperspectral wavelength (400-1000 nm) by days after germination for corn hybrid P998CYXR treated with different fungicide+polymer seed treatments compared to wild-type (not treated seed) at 9 days after emergence.

FIG. 4: Pixel area effect size by days after germination for corn hybrid P13YHRCE treated with different fungicide seed treatments compared to wild-type (not treated).

FIG. 4a: Pixel area effect size by days after germination for corn hybrid P13YHRCE treated with different fungicide seed treatments compared to wild-type (not treated) from 5 to 9 days after emergence.

FIG. 4b: Pixel area effect size by days after germination for corn hybrid P13YHRCE treated with different fungicide+polymer seed treatments compared to wild-type (not treated) from 5 to 9 days after emergence.

FIG. 5: Average standard deviation difference in hyperspectral wavelength (400-1000 nm) by days after germination for corn hybrid P1395YHRCE treated with different fungicide seed treatments compared to wild-type (not treated seed) from 5 to 9 days after emergence.

FIG. 5a: Average standard deviation difference in hyperspectral wavelength (400-1000 nm) by days after germination for corn hybrid P1395YHRCE treated with different fungicide seed treatments compared to wild-type (not treated seed) at 5 days after emergence.

FIG. 5b: Average standard deviation difference in hyperspectral wavelength (400-1000 nm) by days after germination for corn hybrid P1395YHRCE treated with different fungicide seed treatments compared to wild-type (not treated seed) at 7 days after emergence.

FIG. 5c: Average standard deviation difference in hyperspectral wavelength (400-1000 nm) by days after germination for corn hybrid P1395YHRCE treated with different fungicide seed treatments compared to wild-type (not treated seed) at 9 days after emergence.

FIG. 6: Average standard deviation difference in hyperspectral wavelength (400-1000 nm) by days after germination for corn hybrid P1395YHRCE treated with different fungicide+polymer seed treatments compared to wild-type (not treated seed) from 5 to 9 days after emergence.

FIG. 6a: Average standard deviation difference in hyperspectral wavelength (400-1000 nm) by days after germination for corn hybrid P1395YHRCE treated with different fungicide+polymer seed treatments compared to wild-type (not treated seed) at 5 days after emergence.

FIG. 6b: Average standard deviation difference in hyperspectral wavelength (400-1000 nm) by days after germination for corn hybrid P1395YHRCE treated with different fungicide+polymer seed treatments compared to wild-type (not treated seed) at 7 days after emergence.

FIG. 6c: Average standard deviation difference in hyperspectral wavelength (400-1000 nm) by days after germination for corn hybrid P1395YHRCE treated with different fungicide+polymer seed treatments compared to wild-type (not treated seed) at 9 days after emergence.

FIG. 7: Pixel area effect size by days after germination for corn hybrid P9998CYXR treated with different insecticide seed treatments compared to wild-type (not treated).

FIG. 7a: Pixel area effect size by days after germination for corn hybrid P9998CYXR treated with different insecticide seed treatments compared to wild-type (not treated) from 5 to 9 days after emergence.

FIG. 7b: Pixel area effect size by days after germination for corn hybrid P9998CYXR treated with different insecticide+polymer seed treatments compared to wild-type (not treated) from 5 to 9 days after emergence.

FIG. 8: Average standard deviation difference in hyperspectral wavelength (400-1000 nm) by days after germination for corn hybrid P998CYXR treated with different insecticide seed treatments compared to wild-type (not treated seed) from 5 to 9 days after emergence.

FIG. 8a: Average standard deviation difference in hyperspectral wavelength (400-1000 nm) by days after germination for corn hybrid P998CYXR treated with different insecticide seed treatments compared to wild-type (not treated seed) at 5 days after emergence.

FIG. 8b: Average standard deviation difference in hyperspectral wavelength (400-1000 nm) by days after germination for corn hybrid P998CYXR treated with different insecticide seed treatments compared to wild-type (not treated seed) at 7 days after emergence.

FIG. 8c: Average standard deviation difference in hyperspectral wavelength (400-1000 nm) by days after germination for corn hybrid P998CYXR treated with different insecticide seed treatments compared to wild-type (not treated seed) at 9 days after emergence.

FIG. 9: Average standard deviation difference in hyperspectral wavelength (400-1000 nm) by days after germination for corn hybrid P998CYXR treated with different insecticide+polymer seed treatments compared to wild-type (not treated seed) from 5 to 9 days after emergence.

FIG. 9a: Average standard deviation difference in hyperspectral wavelength (400-1000 nm) by days after germination for corn hybrid P998CYXR treated with different insecticide+polymer seed treatments compared to wild-type (not treated seed) at 5 days after emergence.

FIG. 9b: Average standard deviation difference in hyperspectral wavelength (400-1000 nm) by days after germination for corn hybrid P998CYXR treated with different insecticide+polymer seed treatments compared to wild-type (not treated seed) at 7 days after emergence.

FIG. 9c: Average standard deviation difference in hyperspectral wavelength (400-1000 nm) by days after germination for corn hybrid P998CYXR treated with different insecticide+polymer seed treatments compared to wild-type (not treated seed) at 9 days after emergence.

FIG. 10: Average standard deviation difference in pixel area by days after germination for corn hybrid P1395YHCE treated with different insecticide seed treatments compared to wild-type (not treated).

FIG. 10a: Average standard deviation difference in pixel area by days after germination for corn hybrid P1395YHCE treated with different insecticide seed treatments compared to wild-type (not treated) from 5 to 9 days after emergence.

FIG. 10b: Average standard deviation difference in pixel area by days after germination for corn hybrid P1395YHCE treated with different insecticide+polymer seed treatments compared to wild-type (not treated) from 5 to 9 days after emergence.

FIG. 11: Average standard deviation difference in hyperspectral wavelength (400-1000 nm) by days after germination for corn hybrid P1395YHRCE treated with different insecticide seed treatments compared to wild-type (not treated seed) from 5 to 9 days after emergence.

FIG. 11a: Average standard deviation difference in hyperspectral wavelength (400-1000 nm) by days after germination for corn hybrid P1395YHRCE treated with different insecticide seed treatments compared to wild-type (not treated seed) at 5 days after emergence.

FIG. 11b: Average standard deviation difference in hyperspectral wavelength (400-1000 nm) by days after germination for corn hybrid P1395YHRCE treated with different insecticide seed treatments compared to wild-type (not treated seed) at 7 days after emergence.

FIG. 11c: Average standard deviation difference in hyperspectral wavelength (400-1000 nm) by days after germination for corn hybrid P1395YHRCE treated with different insecticide seed treatments compared to wild-type (not treated seed) at 9 days after emergence.

FIG. 12: Average standard deviation difference in hyperspectral wavelength (400-1000 nm) by days after germination for corn hybrid P1395YHRCE treated with different insecticide+polymer seed treatments compared to wild-type (not treated seed) from 5 to 9 days after emergence.

FIG. 12a: Average standard deviation difference in hyperspectral wavelength (400-1000 nm) by days after germination for corn hybrid P1395YHRCE treated with different insecticide+polymer seed treatments compared to wild-type (not treated seed) at 5 days after emergence.

FIG. 12b: Average standard deviation difference in hyperspectral wavelength (400-1000 nm) by days after germination for corn hybrid P1395YHRCE treated with different insecticide+polymer seed treatments compared to wild-type (not treated seed) at 7 days after emergence.

FIG. 12c: Average standard deviation difference in hyperspectral wavelength (400-1000 nm) by days after germination for corn hybrid P1395YHRCE treated with different insecticide+polymer seed treatments compared to wild-type (not treated seed) at 9 days after emergence.

FIG. 13: Pixel area effect size by days after germination for different corn hybrids treated with biological nematicide or biological nematicide+polymer seed treatments compared to wild-type (not treated) from 5 to 9 days after emergence.

FIG. 13a: Pixel area effect size by days after germination for P9998CYXR corn hybrid treated with biological nematicide or biological nematicide+polymer seed treatments compared to wild-type (not treated) from 5 to 9 days after emergence.

FIG. 13b: Pixel area effect size by days after germination for P1395YHRCE (right) corn hybrid treated with biological nematicide or biological nematicide+polymer seed treatments compared to wild-type (not treated) from 5 to 9 days after emergence.

FIG. 14: Average standard deviation difference in hyperspectral wavelength (400-1000 nm) by days after germination for corn hybrid P998CYXR treated with biological nematicide or biological nematicide+polymer seed treatments compared to wild-type (not treated seed) from 5 to 9 days after emergence.

FIG. 14a: Average standard deviation difference in hyperspectral wavelength (400-1000 nm) by days after germination for corn hybrid P998CYXR treated with biological nematicide or biological nematicide+polymer seed treatments compared to wild-type (not treated seed) at 5 days after emergence.

FIG. 14b: Average standard deviation difference in hyperspectral wavelength (400-1000 nm) by days after germination for corn hybrid P998CYXR treated with biological nematicide or biological nematicide+polymer seed treatments compared to wild-type (not treated seed) at 7 days after emergence.

FIG. 14c: Average standard deviation difference in hyperspectral wavelength (400-1000 nm) by days after germination for corn hybrid P998CYXR treated with biological nematicide or biological nematicide+polymer seed treatments compared to wild-type (not treated seed) at 9 days after emergence.

FIG. 15: Average standard deviation difference in hyperspectral wavelength (400-1000 nm) by days after germination for corn hybrid P135YHRCE treated with biological nematicide or biological nematicide+polymer seed treatments compared to wild-type (not treated seed) from 5 to 9 days after emergence.

FIG. 15a: Average standard deviation difference in hyperspectral wavelength (400-1000 nm) by days after germination for corn hybrid P135YHRCE treated with biological nematicide or biological nematicide+polymer seed treatments compared to wild-type (not treated seed) at 5 days after emergence.

FIG. 15b: Average standard deviation difference in hyperspectral wavelength (400-1000 nm) by days after germination for corn hybrid P135YHRCE treated with biological nematicide or biological nematicide+polymer seed treatments compared to wild-type (not treated seed) at 7 days after emergence.

FIG. 15c: Average standard deviation difference in hyperspectral wavelength (400-1000 nm) by days after germination for corn hybrid P135YHRCE treated with biological nematicide or biological nematicide+polymer seed treatments compared to wild-type (not treated seed) at 9 days after emergence.

FIG. 16: Pixel area effect size by days after germination for corn hybrids treated with combinations of fungicide+insecticide+biologicals+polymer seed treatments compared to WT (wild-type not treated control).

FIG. 16a: Pixel area effect size by days after germination for corn hybrid P1138YHR treated with combinations of fungicide+insecticide+biologicals+polymer seed treatments compared to WT (wild-type not treated control) from 7 to 10 days after emergence.

FIG. 16b: Pixel area effect size by days after germination for corn hybrid P1395YHRCE treated with combinations of fungicide+insecticide+biologicals+polymer seed treatments compared to WT (wild-type not treated control) from 7 to 10 days after emergence.

FIG. 17: Average standard deviation difference in hyperspectral wavelength (400-1000 nm) by days after germination for corn hybrid P1138YHR treated with treated with combinations of fungicide+insecticide+biologicals+polymer seed treatments compared to WT (wild-type not treated control) from 7 to 10 days after emergence.

FIG. 17a: Average standard deviation difference in hyperspectral wavelength (400-1000 nm) by days after germination for corn hybrid P1138YHR treated with treated with combinations of fungicide+insecticide+biologicals+polymer seed treatments compared to WT (wild-type not treated control) at 7 days after emergence.

FIG. 17b: Average standard deviation difference in hyperspectral wavelength (400-1000 nm) by days after germination for corn hybrid P1138YHR treated with treated with combinations of fungicide+insecticide+biologicals+polymer seed treatments compared to WT (wild-type not treated control) at 9 days after emergence.

FIG. 18: Average standard deviation difference in hyperspectral wavelength (400-1000 nm) by days after germination for corn hybrid P1395YHRCE treated with treated with combinations of fungicide+insecticide+biologicals+polymer seed treatments compared to WT (wild-type not treated control) from 7 to 10 days after emergence.

FIG. 18a: Average standard deviation difference in hyperspectral wavelength (400-1000 nm) by days after germination for corn hybrid P1395YHRCE treated with treated with combinations of fungicide+insecticide+biologicals+polymer seed treatments compared to WT (wild-type not treated control) at 7 days after emergence.

FIG. 18b: Average standard deviation difference in hyperspectral wavelength (400-1000 nm) by days after germination for corn hybrid P1395YHRCE treated with treated with combinations of fungicide+insecticide+biologicals+polymer seed treatments compared to WT (wild-type not treated control) at 9 days after emergence.

FIG. 19: Pixel area effect size by days after germination for soybean variety P31A22X treated with different fungicide seed treatments compared to wild-type (not treated).

FIG. 19a: Pixel area effect size by days after germination for soybean variety P31A22X treated with different fungicide seed treatments compared to wild-type (not treated) from 5 to 11 days after emergence.

FIG. 19b: Pixel area effect size by days after germination for soybean variety P31A22X treated with different fungicide+polymer seed treatments compared to wild-type (not treated) from 5 to 11 days after emergence.

FIG. 20: Average standard deviation difference in hyperspectral wavelength (400-1000 nm) by days after germination for soybean variety P31A22X treated with different fungicide seed treatments compared to wild-type (not treated seed) from 5 to 11 days after emergence.

FIG. 20a: Average standard deviation difference in hyperspectral wavelength (400-1000 nm) by days after germination for soybean variety P31A22X treated with different fungicide seed treatments compared to wild-type (not treated seed) at 5 days after emergence.

FIG. 20b: Average standard deviation difference in hyperspectral wavelength (400-1000 nm) by days after germination for soybean variety P31A22X treated with different fungicide seed treatments compared to wild-type (not treated seed) at 7 days after emergence.

FIG. 20c: Average standard deviation difference in hyperspectral wavelength (400-1000 nm) by days after germination for soybean variety P31A22X treated with different fungicide seed treatments compared to wild-type (not treated seed) at 9 days after emergence.

FIG. 21: Average standard deviation difference in hyperspectral wavelength (400-1000 nm) by days after germination for soybean variety P31A22X treated with different fungicide+polymer seed treatments compared to wild-type (not treated seed) from 5 to 11 days after emergence.

FIG. 21a: Average standard deviation difference in hyperspectral wavelength (400-1000 nm) by days after germination for soybean variety P31A22X treated with different fungicide+polymer seed treatments compared to wild-type (not treated seed) at 5 days after emergence.

FIG. 21b: Average standard deviation difference in hyperspectral wavelength (400-1000 nm) by days after germination for soybean variety P31A22X treated with different fungicide+polymer seed treatments compared to wild-type (not treated seed) at 7 days after emergence.

FIG. 21c: Average standard deviation difference in hyperspectral wavelength (400-1000 nm) by days after germination for soybean variety P31A22X treated with different fungicide+polymer seed treatments compared to wild-type (not treated seed) at 9 days after emergence.

FIG. 22: Pixel area effect size by days after germination for soybean variety P48A60X treated with different fungicide seed treatments compared to wild-type (not treated).

FIG. 22a: Pixel area effect size by days after germination for soybean variety P48A60X treated with different fungicide seed treatments compared to wild-type (not treated) from 5 to 11 days after emergence.

FIG. 22b: Pixel area effect size by days after germination for soybean variety P48A60X treated with different fungicide+polymer seed treatments compared to wild-type (not treated) from 5 to 11 days after emergence.

FIG. 23: Average standard deviation difference in hyperspectral wavelength (400-1000 nm) by days after germination for soybean variety P48A60X treated with different fungicide seed treatments compared to wild-type (not treated seed) from 5 to 11 days after emergence.

FIG. 23a: Average standard deviation difference in hyperspectral wavelength (400-1000 nm) by days after germination for soybean variety P48A60X treated with different fungicide seed treatments compared to wild-type (not treated seed) at 5 days after emergence.

FIG. 23b: Average standard deviation difference in hyperspectral wavelength (400-1000 nm) by days after germination for soybean variety P48A60X treated with different fungicide seed treatments compared to wild-type (not treated seed) at 7 days after emergence.

FIG. 23c: Average standard deviation difference in hyperspectral wavelength (400-1000 nm) by days after germination for soybean variety P48A60X treated with different fungicide seed treatments compared to wild-type (not treated seed) at 9 days after emergence.

FIG. 24: Average standard deviation difference in hyperspectral wavelength (400-1000 nm) by days after germination for soybean variety P48A60X treated with different fungicide+polymer seed treatments compared to wild-type (not treated seed) from 5 to 11 days after emergence.

FIG. 24a: Average standard deviation difference in hyperspectral wavelength (400-1000 nm) by days after germination for soybean variety P48A60X treated with different fungicide+polymer seed treatments compared to wild-type (not treated seed) at 5 days after emergence.

FIG. 24b: Average standard deviation difference in hyperspectral wavelength (400-1000 nm) by days after germination for soybean variety P48A60X treated with different fungicide+polymer seed treatments compared to wild-type (not treated seed) at 7 days after emergence.

FIG. 24c: Average standard deviation difference in hyperspectral wavelength (400-1000 nm) by days after germination for soybean variety P48A60X treated with different fungicide+polymer seed treatments compared to wild-type (not treated seed) at 9 days after emergence.

FIG. 25: Pixel area effect size by days after germination for soybean varieties treated with different insecticide or insecticide+polymer (right) seed treatments compared to wild-type (not treated) from 5 to 10 days after emergence.

FIG. 25a: Pixel area effect size by days after germination for soybean variety P31A22X treated with different insecticide or insecticide+polymer (right) seed treatments compared to wild-type (not treated) from 5 to 10 days after emergence.

FIG. 25b: Pixel area effect size by days after germination for soybean variety P48A60X treated with different insecticide or insecticide+polymer (right) seed treatments compared to wild-type (not treated) from 5 to 10 days after emergence.

FIG. 26: Average standard deviation difference in hyperspectral wavelength (400-1000 nm) by days after germination for soybean variety P31A22X treated with different insecticide or insecticide+polymer seed treatments compared to wild-type (not treated seed) from 5 to 10 days after emergence.

FIG. 26a: Average standard deviation difference in hyperspectral wavelength (400-1000 nm) by days after germination for soybean variety P31A22X treated with different insecticide or insecticide+polymer seed treatments compared to wild-type (not treated seed) at 5 days after emergence.

FIG. 26b: Average standard deviation difference in hyperspectral wavelength (400-1000 nm) by days after germination for soybean variety P31A22X treated with different insecticide or insecticide+polymer seed treatments compared to wild-type (not treated seed) at 7 days after emergence.

FIG. 26c: Average standard deviation difference in hyperspectral wavelength (400-1000 nm) by days after germination for soybean variety P31A22X treated with different insecticide or insecticide+polymer seed treatments compared to wild-type (not treated seed) at 9 days after emergence.

FIG. 27: Average standard deviation difference in hyperspectral wavelength (400-1000 nm) by days after germination for soybean variety P48A60X treated with different insecticide or insecticide+polymer seed treatments compared to wild-type (not treated seed) from 5 to 10 days after emergence.

FIG. 27a: Average standard deviation difference in hyperspectral wavelength (400-1000 nm) by days after germination for soybean variety P48A60X treated with different insecticide or insecticide+polymer seed treatments compared to wild-type (not treated seed) at 5 days after emergence.

FIG. 27b: Average standard deviation difference in hyperspectral wavelength (400-1000 nm) by days after germination for soybean variety P48A60X treated with different insecticide or insecticide+polymer seed treatments compared to wild-type (not treated seed) at 7 days after emergence.

FIG. 27c: Average standard deviation difference in hyperspectral wavelength (400-1000 nm) by days after germination for soybean variety P48A60X treated with different insecticide or insecticide+polymer seed treatments compared to wild-type (not treated seed) at 9 days after emergence.

FIG. 28: Pixel area effect size by days after germination for soybean varieties treated with combinations of fungicide+insecticide+biologicals+polymer seed treatments compared to WT (wild-type not treated control) from 7 to 14 days after emergence.

FIG. 28a: Pixel area effect size by days after germination for soybean variety P31A22X treated with combinations of fungicide+insecticide+biologicals+polymer seed treatments compared to WT (wild-type not treated control) from 7 to 14 days after emergence.

FIG. 28b: Pixel area effect size by days after germination for soybean variety P72A21X treated with combinations of fungicide+insecticide+biologicals+polymer seed treatments compared to WT (wild-type not treated control) from 7 to 14 days after emergence.

FIG. 29: Average standard deviation difference in hyperspectral wavelength (400-1000 nm) by days after germination for soybean variety P31A22X treated with combinations of fungicide+insecticide+biologicals+polymer seed treatments compared to wild-type (not treated seed) from 7 to 14 days after emergence.

FIG. 29a: Average standard deviation difference in hyperspectral wavelength (400-1000 nm) by days after germination for soybean variety P31A22X treated with combinations of fungicide+insecticide+biologicals+polymer seed treatments compared to wild-type (not treated seed) at 7 days after emergence.

FIG. 29b: Average standard deviation difference in hyperspectral wavelength (400-1000 nm) by days after germination for soybean variety P31A22X treated with combinations of fungicide+insecticide+biologicals+polymer seed treatments compared to wild-type (not treated seed) at 9 days after emergence.

FIG. 30: Average standard deviation difference in hyperspectral wavelength (400-1000 nm) by days after germination for soybean variety P72A21X treated with combinations of fungicide+insecticide+biologicals+polymer seed treatments compared to wild-type (not treated seed) from 7 to 14 days after emergence.

FIG. 30a: Average standard deviation difference in hyperspectral wavelength (400-1000 nm) by days after germination for soybean variety P72A21X treated with combinations of fungicide+insecticide+biologicals+polymer seed treatments compared to wild-type (not treated seed) at 7 days after emergence.

FIG. 30b: Average standard deviation difference in hyperspectral wavelength (400-1000 nm) by days after germination for soybean variety P72A21X treated with combinations of fungicide+insecticide+biologicals+polymer seed treatments compared to wild-type (not treated seed) at 9 days after emergence.

### DETAILED DESCRIPTION

For the purpose of promoting an understanding of the principles of the methods disclosed herein, reference will now be made to various aspects of the disclosed methods and specific language will be used to describe the same. It will nevertheless be understood that no limitation of the scope of the claimed methods is thereby intended. Any alterations and further modifications in the described aspects, and any further applications of the principles of the disclosed methods as described herein are contemplated as would normally occur to one skilled in the art to which the claimed method relates.

A high throughput method of obtaining, analyzing and processing data into meaningful results is crucial in any crop cultivation program, especially with respect to seed treatment protocols. Generally, the seeds are treated with one or more appropriate seed treatment protocols, and the effectiveness of the treatment protocols related to a desired outcome, such as plant growth, elimination of pests and pathogens, improving yield etc., needs to be determined in a time-efficient and reliable manner. This includes collection, analysis, and processing of large amounts of data.

In one aspect, the disclosed methods are related to evaluating the efficacy of one or more seed treatment protocols. Seed treatments have emerged as a powerful and efficient treatment method where the seeds are treated with an appropriate treatment and the plants are grown from the treated seeds. Plants grown from the treated seeds manifest the desired outcomes, for example as prevention or elimination of pest and/or pathogen infestations. In this regard, insecticides, fungicides, nematicides and other pesticides have shown excellent results.

Pathogen/pest control and management continue to evolve as these harmful organisms develop resistance to the existing treatments. More effective treatments are continually being developed. As the new treatments emerge, it is important to obtain the effectiveness of such treatments and improve, customize and modify them. Generally, if the data can be obtained before implementing the treatment protocols in a larger scale, significant savings in time and cost can be realized. It also provides a greater certainty with respect to the desired outcome of a given treatment protocol.

Treating a seed with plant treatment and obtaining data related to the treatment using a plant grown from such a seed involve collecting, analyzing, and processing large amounts of data. Often more than one seed treatments need to be compared and analyzed to determine which treatment(s) are suitable for a particular situation (e.g., crop, pest, climate, soil pattern etc.,). Accordingly, a high throughput method capable of obtaining precise data in a time-efficient manner is continually being sought by the farming community.

Seed treatments can be conducted in a field-setting. Often, the field evaluations of a plant treatment protocol are conducted by different individuals at different time intervals by assigning visual ratings to differences in emergence and early vigor of a treated seed in comparison to those of not treated seed. As several variables are involved in these field evaluations, these visual ratings are inconsistent and have a high level of human error. It is not possible to conduct such visual field evaluations in a high throughput manner.

Spectral-based evaluation methods have increased the precision and improved the efficiency. For example, Unmanned Aerial Vehicles (UAV) equipped with cameras and spectral sensors have greatly improved the precision of collecting phenotypic data in field testing (Maes and Steppe, 2019; Milics, 2019; Olson and Anderson, 2021). Several UAV systems have been developed for data collection (Puri et al., 2017; Olson and Anderson, 2021). Techniques for converting a digital image photo into phenotypic data have been developed (Lee and Lee, 2011; Zhao et al., 2018; Daponte et al., 2019; Narayanan et al., 2019; Olson and Anderson, 2021). UAVs have found increasing use in vegetation characterization and mapping, land management, environmental monitoring, and plant phenotype characterizations.

However, UAV evaluation methods are often costly, and often require a rigorous calibration process. The mathematical analysis required to process the imagery data can be expensive and carries a high computational cost.

More importantly, UAV evaluation methods are only applicable in field testing. Field testing conditions take considerable time, and highly dependent on the seasonal conditions.

A greenhouse setting where all variables are being controlled, and where the cultivation of crops can be carried out throughout the year independent of the seasonal conditions is an alternative to a field testing. However, data collection methods used in a field setting, for example UAV-based data collection methods, cannot be adaptable to a greenhouse setting for various reasons. Accordingly, greenhouse data collection and analysis are largely based on visual inspection of the plant response. The observer visually inspects the plant response that is visible, and assigns a suitable rating to the plant response. The visual ratings can be highly subjective and variable, and cannot be used in a high throughput manner.

High throughput evaluation techniques that do not depend on the visual observations greatly reduce the human error, inconsistency, and time and effort. In this regard, discovery of a high throughput method that could detect changes that are not visible to the human eye, precise and efficient is highly warranted. Such a method must also be completely automated, which can be self-programmable or programmable by an end user.

Disclosed herein is a computer-implemented, total reflectance spectral-based high throughput method for evaluating the efficacy of a seed treatment protocol that can be conducted in a greenhouse setting.

Any seed treatment protocol that is known in the art can be used with the disclosed high throughput total reflectance spectral-based evaluation method. The seed treatment can be one or more of a biological or chemical treatment related to pest or pathogen prevention or elimination treatment. In some instances, it is a fungicide treatment. In other instances, it is an insecticide treatment and in certain other instances, it is a nematicide treatment. The seed treatment protocol can also be related to a treatment related to enhance or optimize plant nutrition. For example, in some instances, the plant treatment protocol may comprise plant growth promoters, plant stimulants, other components that can enhance growth, yield, nutrition, health of the plants. The treatments can be used alone or in combinations with each other. For example, fungicides, insecticides, and/or nematicides can be used individually, or in combination with each other. Accordingly, some seed treatments comprise one or more fungicides, one or more insecticides or one or more nematicides, alone or in combination with each other. For example, some seed treatments may comprise one or more fungicides. Others may comprise one or more insecticides. Some others may comprise one or more nematicides. In some case, seed treatments may comprise one or more fungicide, insecticide and/or nematicide and any combination thereof. The actives can be both biological agents and chemical agents. For example, in some instances, a seed treatment comprises a biological nematicide and a synthetic fungicide. In some instances, it can be an amendment, which may act as a component to enhance germination, growth, and vigor (such as a fertilizer or micronutrient) or aid the plant in improving germination and early growth by enabling more efficient nutrient or water uptake. Amendment means any material (a biological material, naturally-occurring substance, synthesized chemical compound and the like) that can be added to the seed and/or soil adjacent to seed to improve seed germination and/or plant growth characteristics.

### Fungicides

Fungicides that can be used with the disclosed methods can be Aldimorph, ampropylfos, ampropylfos-potassium, andoprim, anilazin, azaconazole, azoxystrobin, benalaxyl, benodanil, benomyl, benzamacril, benzamacryl-isobutyl, bialaphos, binapacryl, biphenyl, bitertanol, blasticidin-S, bromuconazole, bupirimate, buthiobate, calcium poly sulfide, capsimycin, captafol, captan, carbendazim, carboxin, carvone, quinomethionate, chlobenthiazon, chlorfenazole, chloroneb, chloropicrin, chlorothalonil, clothianidin, chlozolinate, clozylacon, cufraneb, cymoxanil, cyproconazole, cyprodinil, cyprofuram, debacarb, dichlorophen, diclobutrazol, diclofluanid, diclomezin, dicloran, diethofencarb, difenoconazole, dimethirimol, dimethomorph, diniconazole, diniconazole-M, dinocap, diphenylamine, dipyrithione, ditalimfos, dithianon, dodemorph, dodine, drazoxolon, ediphenphos, epoxiconazole, etaconazole, ethirimol, ethaboxam, etridiazole, famoxadon, fenapanil, fenarimol, fenbuconazole, fenfuram, fenitropan, fenpiclonil, fenpropidin, fenpropimorph, fentin acetate, fentin hydroxide, ferbam, ferimzon, fluazinam, fludioxonil, flumetover, fluoromid, fluquinconazole, fluopyram, fluoroprimidol, flusilazole, flusulfamide, flutolanil, flutriafol, fluxapyroxad, fluoxastrobin, folpet, fosetylaluminium, fosetylsodium, fthalide, fuberidazole, furalaxyl, furametpyr, furcarbonil, furconazole, furconazole-cis, furmecyclox, guazatine, hexachlorobenzene, hexaconazole, hymexazole, imazalil, imibenconazole, imidacloprid, iminoctadine, iminoctadine-albesilate, iminoctadine-triacetate, inpyrfluxam, iodocarb, ipconazole, iprobenfos (IBP), iprodione, irumamycin, isoprothiolane, isovaledione, kasugamycin, kresoxim-methyl, copper preparations such as: copper hydroxide, copper naphthenate, copper oxychloride, copper sulfate, copper oxide, oxine-copper and Bordeaux mixture, mancopper, mancozeb, maneb, meferimzone, mepanipyrim, mepronil, metalaxyl, metconazole, methasul-focarb, methfuroxam, mefenoxam, metiram, metomeclam, metsulfovax, mildiomycin, myclobutanil, myclozolin, nickel dimethyldithiocarbamate, nitrothal-isopropyl, nuarimol, ofurace, oxadixyl, oxamocarb, oxathiapiprolin, oxolinic acid, oxycarboxim, oxy-fenthiin, paclobutrazole, pefurazoate, penconazole, pencycuron, penfluten, phosdiphen, picoxystrobin, pimaricin, piperalin, polyoxine, polyoxorim, probenazole, prochloraz, procymidon, propamocarb, propanosine-sodium, propiconazole, propineb, prothioconazole. pyrazophos, pyrifenox, pyrimethanil, pyroquilon, pyroxyfur, pyraclostrobin, quinconazole, quintozene (PCNB), sulfur and sulfur preparations, sedaxane, tebuconazole, tecloftalam, tecnazene, tetcyclacis, tetraconazole, thiabendazole, thicyofen, thifluzamide, thiophanate-methyl, thiram, tioxymid, tolclofos-methyl, tolylfluanid, triadimefon, triadimenol, triazbutil, triazoxide, trichlamide, triconazole and zoxamide.

### Insecticides/Acaricides/Nematicides

Insecticides that can be used with the disclosed methods can be brofanilide, chlorantraniliprole, clothianidin, cyantraniliprole, imidacloprid, spinosad, spinetoram, sulfoxaflor, tefluthrin, thiamethoxam, triflumexopyrim, abamectin, fluopyram, thiodicarb, and tioxazafin, or biologicals with nematicide properties selected from Bacillus species and Burkholderia species.

### Amendment/Biological or Plant Stimulant

Biological actives that can be used with the disclosed methods can be Acinetobacter species, Arthrobacter species. Bacillus species, Bradyrhizobium species, Buricholderia species, Burkholderia species, Carnobacterium species, Flavobacterium species, Kosakonia species, Methyl bacterium species, Pantoea species, Pasteuria species, Penicillium species, Pseudomonas species, Rahnella species, Rhizobium species, Sphingobium species, Stenotrophomonas species, Trichoderma species, and Variovorax species.

In one key aspect, the disclosed methods herein are used in a greenhouse setting allowing farmers/researchers to collect precise data in a time efficient manner. The method comprises first, subjecting a plurality of seeds to a seed treatment protocol. The disclosed method can be used with any type of seed-bearing crop. In some aspects, without limitation, the crop is alfalfa, barley, canola, cereal, corn, cotton, maize, pulse crops, soybean, sorghum, oilseed rape, sunflower, wheat and/or vegetables.

In some other aspects, the crop is corn. In one aspect, the crop is soybean. In one aspect, the crop is a cereal, such as sorghum, millet, wheat, maize or rice.

According to the methods described herein, some seeds are not treated (Wild-type seeds) and used as controls such that the plant response from a plant grown from a treated seed and a plant grown from a not treated seed can be compared.

The plurality of treated seeds is then allowed to germinate into plants. The Wild-type seeds are subjected to the same growing conditions. The plant response is then measured at a pre-determined time. The pre-determined time is measured as Days After the Emergence (DAE). DAE is defined as between the V0 to V6 growth stages. In some instances, the plant response is measured at 5 DAE, 6 DAE, 7 DAE, 8 DAE, 9 DAE, 10 DAE, 11 DAE, 12 DAE, 13 DAE, or 14 DAE. The plant response can be measured up to 30 DAE, depending on the crop and the growth conditions. In some instances, the plant response is measured at any time between 5 DAE and 14 DAE. In other instances, the plant response is measured at any time between 6 DAE and 14 DAE. In certain instances, the plant response is measured at any time between 7 DAE and 14 DAE. In certain other instances, the plant response is measured at any time between 8 DAE and 14 DAE. As known to a person skilled in the art, the growth stages can vary depending on the type of crop/plant and these measurement timepoints in days correspond with any plant/crop having a vegetative growth stage between V0 and V6. V0 to V6 plant vegetative stages are well-known terms in the field of agriculture. For example, corn and soybean growth stages can be classified as vegetative or reproductive. The corn vegetative stages are based upon the number of visible leaf collars, starting with emergence (VE). Each new leaf with a fully-developed leaf collar is a V stage (for example, V1 has 1 fully-developed leaf with a collar, V2 has 2 fully-developed leaves with collar). The soybean vegetative stages are numbered according to how many fully-developed trifoliate leaves are present, starting at emergence (VE), as unrolled unifoliate (VC), unfolded first trifoliate (V1), unfolded second trifoliate (V2) and the like.

The plant response is measured using a high throughput spectral method. The measurement comprises measuring one or more indication related to the desired outcome of the seed treatment protocol that is manifested in a plant grown from a treated seed. In some instances, the indication is one or more of plant phenotype, plant growth, plant health, or plant vigor. These indications or plant responses can be measured using any objective measurement that correlates with one or more of one or more of plant phenotype, plant growth, plant health, or plant vigor. For example, in some instances, the objective measurement is one or more of pixel area, green index, yellow index, brown index, spectral signature, vegetative index, Normalized Difference Vegetation Index (NDVI), Green biomass Density (GBD), Green Leaf Area Index (GLAI), Absorbed Photosynthetically-Active Radiation (APAR), and Gross Primary Production (GPP).

With respect to the seed treatments that comprise prevention and/or elimination of certain pests and/or pathogens, the plant response of the plants grown from the seeds treated with seed treatment can be anyone of plant phenotype, plant growth, plant health, or plant vigor. For example, if certain pests and/or pathogens are reduced or eliminated, the resulting plants may show indications of good health, growth and/or plant vigor. The plant response can be measured using any of the objective measurements below:
i) Pixel area—total area of the plant as determined by imaging angle in pixels
ii) Green index—intensity of the green signals from each pixel relative to total area
iii) Yellow index—intensity of the yellow signals from each pixel relative to total area
iv) Brown index—intensity of the brown signals from each pixel relative to total area
v) Spectral signature—unique pattern of wavelength reflectance for each experimental unit or area of interest, including but not limited to individual plants, treatments, subset of treatments, subset of plants, or parts of plants such as leaves or stems.
vi) Vegetative index—a measurement of plant material and biomass
vii) Normalized difference vegetation index—a measurement of plant material and biomass using a ratio of the NIR and red wavelengths
viii) Green biomass density—a measurement of plant material and biomass in terms of biomass per unit area
ix) Green leaf area index—a measurement of plant material and biomass in terms of green leaf area per unit of ground area
x) Absorbed photosynthetically active radiation (PAR)—a measurement of plant material and biomass in terms of a ratio between available PAR and PAR absorbed by photosynthesis
xi) Gross primary production—a measurement of plant material and biomass in terms of the amount of carbon fixed during photosynthesis

A spectral method that can obtain the data from the grown plants in combination with an appropriate processing program that can covert the data to meaningful indications of plant phenotype, plant growth, plant health or vigor can be used with the presently disclosed methods.

The disclosed methods herein relate to computer-implemented high throughput spectral methods that allow efficient and precise data collection, analysis and output. In particular, the disclosed methods relate to a high throughput spectral methods that use total reflectance spectral analysis methods. Total reflectance spectral analysis is a method by using interpolated reflectance intensities of every wavelength in a range of wavelengths to identify regions of the spectrum that contribute to overall differences. These regions identified may be as narrow as individual bands or as broad as the entire region detected using the specific sensor or camera used to acquire the image or reflectance spectra.

In some aspects, the total reflectance spectral method is selected from one or more of hyperspectral imaging, multispectral imaging, near infra-red imaging, high resolution photography, thermal imaging, optical spectroscopy, RGB spectral imaging, and chlorophyll fluorescence spectral imaging.

It is critical to choose an appropriate total reflectance spectral method that is suitable for high throughput data collection. Certain modifications must be made to the method of analysis such that identification of regions of the spectrum that contribute to overall differences can be identified reliably.

In a particular aspect, the high throughput total reflectance spectral method is hyperspectral imaging.

### Hyperspectral Imaging

### i) Introduction

Considerable efforts have been dedicated to identifying a suitable method that can diagnose plant diseases before the onset, and before the visible signs appear. Such a method must be precise in detecting specific regions that are affected by a particular disease, and capable of detecting even subtle changes in plants under disease conditions. Hyperspectral imaging has been proposed as a preferable technique to identify disease conditions (Mahlein A-K. disease detecting by imaging sensors-parallels and specific demands for precision agriculture and plant phenotyping. Plant. Dis. 2016; 100:241-51.)

Image analysis involves the use of computational techniques to extract information from digital images. This comprises capturing images and automatic processing of the captured images to produce a dataset of measurements. Hyperspectral imaging is a technologically advanced method of image capturing, which includes cameras capturing more than the usual three bands of colored light found in the traditional digital imaging. Hyperspectral imaging uses high-fidelity color reflectance information over a large range of the light spectrum, and thus useful for identifying subtle changes in the plant growth and development.

The section of the spectrum that is used for hyperspectral imaging of plants ranges from ultraviolet (UV) starting ~250 nm up to short-wave infrared (SWIR) ~2500 nm (cf. with color visible to human eye from 400-700 nm). Hyperspectral images typically contain hundreds of contiguous narrow wavelength bands over a spectral range. This approach produces a dense, information rich color dataset with enough spatial resolution to have many hundreds of data points (pixels) per leaf.

In a hyperspectral spectrometer, there are different means of capturing the image; for example, the operation includes push broom, filter wheel, liquid crystal turntable among others. Hyperspectral imaging can be used in laboratory-based settings as well as field-based setting. The chosen setting has an impact of the hyperspectral image itself. For example, in a field setting, aerial remote sensing is typically used, and the spatial resolution is typically in the range of meters per pixel. As such, the pixels usually contain signatures from more than one material. In this scenario, the pixels must be considered as containing mixed materials (called mixed pixels), and a spectral unmixing process must be applied. A single pixel may contain plant and solid, and algorithms must be used to determine the appropriate mix.

Hyperspectral devices in general do not provide a point-and-click measurement. Instead, the data must be acquired, and the resulting large numerical datasets must be analyzed in order to provide useful information. This can be done by considering only a small number of positions in the wavelength range, looking at changes across conditions at predetermined key points in the spectrum. This approach involves combining two or more wavelengths, commonly known as “indices.”

To interpret the data, a number of such indices have been developed, through either pre-considered biological reasoning (e.g., knowing that a particular wavelength relates to particular cell structure) or due to limitation in the particular wavelengths available from the capture equipment. When applied to plant materials, these indices are known as “vegetation indices.” Many different vegetation indices exist, and each uses a different set of wavelength measurement for describing physiological attributes of vegetation, looking at either general properties of the plant, or at specific parameters of its growth. One of the most popular and widespread metrics is the normalized difference vegetation index (NDVI), which is used for measuring the general health status of crops. It is calculated via a simple ratio of near-IR and visible light. Most of the indices are very specific and only work well with the datasets for which they were designed.

Hyperspectral imaging has been used to detect a particular disease, and to distinguish between different diseases to identify specific pathogens. It has also been used quantify the severity of diseases. Hyperspectral imaging has been used mostly in detecting biotic and abiotic stress in the plants.

One aspect of the high throughput analytical method disclosed herein involves modifying the image collection process such that all plant responses that can be detectable are detected in a reliable manner. In particular, the image collection process needs to capture the spectral details that correlate with the plant response efficiently and in a precise manner. In one aspect, the wavelength range of 250 nm-2500 nm that captures wavelengths beyond the visible spectrum has been used. In another aspect, wavelength range of 400 nm-2500 nm has been used. Multiple cameras that can capture the wavelengths within this range have been used. Accordingly, in one aspect, a specific spectral interpolation is used to enhance spectral resolution in the specified regions to enrich data sets and enable additional calculations and improve statistical models derived from these spectral data. In some specific aspects, wavelength region of 400 nm-2500 nm and a spectral interpolation of 1.0 nm-4.0 nm have been used.

In one particular aspect of the method, multiple cameras have been used. A set of cameras that can measure visible and near IR spectral region (VNIR) of 400 nm-1000 nm, and another set of cameras that can measure short wave IR spectral region (SWIR) of 900-2500 nm have been used. In the VNIR region of 400 nm-1000 nm, increments of 1.0-2.0 nm have been used. In particular embodiments, increments of 1.5 nm have been used. In the SWIR region of 900-2500 nm, increments of 2.0-4.0 have been used. In particular embodiments, increments of 3.5-4.0 nm have been used. In some other embodiments, increments of 3.75 nm have been used. A wide variety of plant physiology and plant responses related thereto have active signals in the VNIR and SWIR ranges. Decreasing the aggregation of signals across this region increases the specificity possible for model building and derivation of underlying physiological processes and accuracy and precision of resultant models and derived features.

### ii) Hyperspectral Imaging Processing

Involves converting the raw, three-dimensional reflectance data matrix collected from a hyperspectral camera (a “hypercube”) into data points that correspond to differences of interest of a particular experiment or set of experiments. Multiple steps are required for image processing, including but not limited to data capture, metadata annotation, model building, image reconstruction, and statistical analysis.

One aspect of the high throughput spectral method is measuring the plant response of a seed treated with a seed treatment. In this respect, the plant response is measured in comparison to a control. In this case, the control is a plant grown from a seed that is not treated with a seed treatment, and grown under the same conditions (called a “Wild-type”). In one particular example, a plant is grown from a seed treated with a seed treatment, and the response to the seed treatment as it is manifested in the plant is measured (X). A plant is grown under the same conditions from a seed that is not treated, and subjected to the same spectral measurements and the plant response is measured (Y). Then the difference between the plant response of a plant grown from a treated seed (X) and that of a plant grown from a not treated seed (Y) is calculated in terms of statistical measurement. The statistical measurement method is selected from the group consisting of Mahalanobis distance, Bhattacharya distance, the Wasserstein metric, or Kolmogorov-Smirnov test. In such statistical measurement methods, if the resulting value of each treatment is more than or equal to a determined significance threshold, then those results are considered for further evaluation. “A determined significance threshold” is a value above which is considered to be indicative of a biologically-relevant significance. This value is dependent on which statistical measurement method is being used.

One particular aspect of the computer-implemented high throughput spectral method disclosed herein comprises the following steps; subjecting a plurality of seeds to a seed treatment protocol and then allowing the plurality of seeds to germinate into plants. The plant response to the seed treatment protocol at a pre-determined growth stage is measured using a reflectance spectral analysis method encompassing the wave length range of 400-2500 nm using reflectance spectra interpolation to derive discrete wavelength measurements between 1-4 nm resolution. The efficacy of the seed treatment protocol is then determined using the measured plant response. In some aspects, the plant response is measured at 400-1000 nm range at 1-2 nm increments and 900-2500 range at 2-4 nm increments. In some other aspects, the plant response is measured at 400-100 nm range at 1.5 nm increments and 900-2500 nm range at 3.75 nm increments. Responses are measured both directly and indirectly, through pixel counting, statistical modeling, supervised learning machine models, and unsupervised learning machine models. Results are also compared accordingly with relevant statistical methods including but not limited to distance analytics (e.g. Euclidian, Mahalanobis), null hypothesis testing, and Bayesian statistics. Often, it is of interest to provide a comparison between a commercially available seed or a plant treatment with a new or modified seed treatment protocol to understand the benefits of such a modified or new seed treatment protocol. Accordingly, in one aspect, a seed is treated with a commercial seed treatment T1 and used as a control. And a seed is treated with a seed treatment protocol T2, for example, modified or a new seed treatment protocol, and a difference between the plant response between a plant grown from a seed treated with a commercial seed treatment protocol (T1) and a plant grown from a seed treated with seed treatment protocol T2 is calculated in terms of a statistical measurement. The statistical measurement method is selected from the group consisting of Mahalanobis distance, Bhattacharya distance, the Wasserstein metric, or Kolmogorov-Smirnov test and wherein resulting value of each treatment is more than or equal to a determined significance threshold.

One such statistical measurement that is used with disclosed methods is Mahalanobis distance. The Mahalanobis distance is a measure of the distance between a datapoint and the distribution of data; quantifying the number of standard deviations the datapoint is away from the mean of the distribution. The larger the value, the further away the seed treatment is from the wild-type not treated control. In some instances, the Mahalanobis distance (log transformation) is at least 3.0. In other instances, the Mahalanobis distance (log transformation) is at least 4.0. In some other specific instances, the Mahalanobis distance (log transformation) is at least 5.0.

A key feature of any evaluation method is being able to evaluate several treatment protocols simultaneously. Such a method must be capable of obtaining a large amount of precise data in a high throughput manner. The disclosed high throughput total reflectance spectral analysis methods gather and process large amounts of precise data. The high throughput spectral methods disclosed herein allows the farmers and researchers to conduct multiple seed treatments and make a determination regarding a particular seed treatment. In this regard, a determination involves conducting one or more seed treatments, and based on the efficacy as determined using the disclosed method, approving one or more treatments for the large-scale use. In some aspects, the determination includes comparing one or more seed treatments with another seed treatment(s). In some other aspects, the determination includes rejection of one or more seed treatments based on the efficacy results obtained using the disclosed methods. The determination also includes an amendment of one or more of seed treatment methods based on the efficacy results. In some instances, the determination includes contrasting or differentiating one or more seed treatments. One or more seed treatments can be conducted, and results can be used to select certain treatments, and either discard or combine certain seed treatments. The currently disclosed methods can also be used to predict an outcome of a seed treatment method or quantification of an outcome of a seed treatment.

One important aspect of the high throughput total spectral analysis methods described herein is the ability of these methods to rapidly screen one or more seed treatment protocols. Currently available methods (for example, visual screening) are not amenable to quickly screen one or more seed treatment protocols. Additionally, the available methods fail to detect responses that are either not visible to a naked eye or require sensitive detection methods. Rapid screening allows the researchers and farmers to make crucial decisions regarding seed treatments before important events occur (e.g., onset of a disease; before the planting season begins etc.,).

Disclosed herein are also methods of obtaining data from a plant grown from a seed treated with one or more of seed treatment. In some instances, the seed treatment is one or more of an amendment, biological, fungicide, an insecticide, or a nematicide. In some instances, the seed treatment is a combination of one or more amendment, biological, fungicide, insecticide or a nematicide. In some instances, the seed treatment is using different rates or amounts of an amendment, biological, fungicide, an insecticide or a nematicide. A seed or a plurality of seeds is treated with a seed treatment as disclosed herein, and data is obtained using the high throughput total reflectance spectral analysis methods. In some aspects, the high throughput total reflectance spectral analysis method is the hyperspectral imaging. The method of obtaining data comprises treating a plurality of seeds with a one or more of seed treatments, and growing plant from the treated seeds. An untreated seed is used as a control, and a plant is grown under identical conditions. The plants are grown, and the plant response is measured at a pre-determined time after the emergence. Preferably, the response is measured 5 days after to 9 days after the emergence. In some instances, the response is measured anytime between 4 days after the emergence and 14 days after the emergence. In some instances, the response is measured anytime between 5 days after the emergence to 30 days after the emergence, corresponding to between the V0 and V6 growth stage.

The data obtained from the disclosed methods can then be used:
a) To differentiate one seed treatment from another seed treatment. The seed treatment comprises one or more of fungicide, insecticide, nematicide, biological active, or any treatment amendment, revision, combination of different seed treatment actives at different rates and the like. In some instances, the data is used to differentiate between one or more formulations, use of different adjuvants and/or formulation auxiliaries;
b) To quantify a plant response of a plant grown from a treated seed. The plant responses vary and differ in type and degree based on the type of seed treatment. The data is used to quantify such a plant response. For example, pixel area can be used to quantify the plant growth in response to different seed treatments;
c) To contrast a seed treatment from another seed treatment. Often researchers and farmers evaluate several seed treatment concepts. The disclosed methods enable comparing several seed treatments based on the efficacy data and making decisions regarding the seed treatments; and
d) Use of anyone or more of the above, a), b) or c) as a screening tool for testing, advancing, discarding, amending or revising a seed treatment.

Disclosed herein is also a computer-implemented method for evaluating the efficacy of a seed treatment, that comprises using hyperspectral imaging to obtain the data from a plant grown for a seed treated with a seed treatment. In some instances, the computer-implemented method is fully-automated and adapted to operate in a greenhouse setting. The computer-implemented method comprises receiving hyperspectral imagery of a plant grown from a seed, processing the hyperspectral imagery using one or more processors to covert the imagery to meaningful information such as pixel area, green index or brown index, and determining the plant response.

The disclosed computer-implemented method herein is used to obtain data related to a plant response of a seed treated with one or more of seed treatments. The seed treatment comprises one or more of an amendment, biological, fungicide, insecticide, nematicide, or combination of different seed treatments, different rates of amendment, biological, fungicide, insecticide, and/or nematicide active components. The data related to the plant response is obtained using hyperspectral analysis and the method comprises, first, receiving hyperspectral imagery of a plant grown from a seed treated with one or more of a seed treatment, and then processing the hyperspectral imagery using a series of image processing algorithm to obtain data that correlates with one or more of a plant phenotype, plant growth, plant health and/or vigor. The data obtained can then be used a) differentiate a seed treatment from another seed treatment b) quantify a plant response of a plant grown from a seed treated with one or more of seed treatments c) contrast one seed treatment with another seed treatment and d) use any of a), b) and/or c) as a screening tool for testing, advancing, discarding, amending or revising one or more seed treatments.

Disclosed herein are also a one or more systems for obtaining data related to a plant response of a plant grown from a seed treated with one or more seed treatments. In some instances, it is a system for obtaining hyperspectral imagery of a plant grown from a seed treated with one or more of a seed treatment and comprising means for obtaining hyperspectral images, means to process the hyperspectral images, and means to read-out the processed data.

In some other instances it is a system for evaluating an efficacy of a seed treatment comprising one or more processors, one or more memory units, one or more non-transitory computer-readable storage media storing one or more computer instructions which, when executed by the one or more processors, cause the one or more processors to perform processing of hyperspectral imagery to obtain data that correlates with one or more of plant phenotype, plant growth, plant health and/or vigor of a plant grown from a seed treated with one or more of amendment, biological, fungicide, insecticide, nematicide seed treatments and/or combinations of different seed treatments, different rates of amendment, biological, fungicide, insecticide and/or nematicide active components. The data obtained from such a system can then be used to make determinations regarding one or more of seed treatments as described throughout this disclosure.

In some other instances it is a system for conducting one or more of the following:
a) To differentiate one seed treatment from another seed treatment. The seed treatment comprises one or more of fungicide, insecticide, nematicide, biological active, or any treatment amendment, revision, combination of different seed treatment actives at different rates and the like. In some instances, the data is used to differentiate between one or more formulations, use of different adjuvants and/or formulation auxiliaries;
b) To quantify a plant response of a plant grown from a treated seed. The plant responses vary and differ in type and degree based on the type of seed treatment. The data is used to quantify such a plant response. For example, pixel area can be used to quantify the plant growth in response to different seed treatments;
c) To contrast a seed treatment from another seed treatment. Often researchers and farmers evaluate several seed treatment concepts. The disclosed methods enable comparing several seed treatments based on the efficacy data and making decisions regarding the seed treatments; and
d) Use of anyone or more of the above, a), b) or c) as a screening tool for testing, advancing, discarding, amending or revising a seed treatment;
   wherein the system comprising one or more processors, one or more memory units, one or more non-transitory computer-readable storage media storing one or more computer instructions which, when executed by the one or more processors, cause the one or more processors to perform processing of hyperspectral imagery encompassing the wavelength range from 250 nm to 2500 nm and using reflectance spectra interpolation of discrete wavelength measurements between 1.0 to 4.0 nm to obtain data that correlates with one or more of plant phenotype, plant growth, plant health and/or vigor of a plant grown from a seed treated with one or more of amendment, biological, fungicide, insecticide, nematicide seed treatments and/or combinations of different seed treatments, different rates of amendment, biological, fungicide, insecticide and/or nematicide active components. The processing also includes the processing of hyperspectral imagery obtained from a plant grown from an untreated seed (wild-type or control). The one or more processors, upon instructions by a processing unit, is capable of comparing, contrasting or distinguishing the plant response of that of a plant grown from the treated seed and a wild-type plant, and produces output in terms of a statistical difference between a plant grown from a treated seed and wild-type plant. In some aspects, such a system can be programmed to run autonomously. For example, such a system can be programmed so that it can be run without any human intervention for a set period of time as desired by the end user.

### EXPERIMENTAL SECTION

### General Procedure:

Experiments were planted in a greenhouse facilities, with two different planting dates of 10 replicates per seed treatment in each planting date. Seed was planted at a standard planting depth of 20 to 25 mm in a standard germination mix similar to 80% peat, 20% perlite, 5 lbs/yd3 lime, 3 lbs/yd3 starter fertilizer, and 0.5 lbs/yd3 wetting agent. The individual seeds were placed in flats of 32 plants each in an 8×4 configuration resulting in a 212 plants/m2 density.

The plants were grown in a glass greenhouse with of a day length of 16 hours and growth conditions of daytime temperature between 21-23 °C, and nighttime temperature between 18-20 °C. Plants were imaged at first visual sight of the plumule using a Hyperspectral Visible and Near InfraRed camera and a short-wave infrared camera. The cameras capture reflectance between 400 nm and 2500 nm and bands were interpolated to 1.5 nm and 4 nm spectral resolution.

Plants grown under normal conditions were imaged daily from 3 days after emergence (DAE) until approximately 9 DAE (V0 to V3 growth stage). Data were processed as below into average reflectance spectra of individual plants and physiologically relevant features. These data were standardized and processed using R (R is a statistical software package and coding language). Custom algorithms were developed to adjust data and remove variability. Data were processed using a modified version of Mahalanobis distance to determine differences between treatments. Resulting distance matrices were log-transformed, with values over 3 considered significantly different.

### Example 1: Corn Fungicide Seed Treatments

### Materials and Methods:

Corn commercial hybrids P9998CYXR and P1395YHR were selected based upon unique genetic background and adaptation to different regions of the United States. Corn hybrid seed were treated with unique fungicide seed treatments (FST) with and without a polymer (Table 1). Seeds were treated as water-based slurries using a Hege 11 laboratory seed treater (Wintersteiger AG, Ried im Innkreis, Austria). Slurries were created by mixing the treatment components in a sufficient volume of tap water to ensure uniform seed coverage. Prepared slurries were pipetted onto the spinning disk of a Hege 11 seed treater in application volumes of 90-130 mL slurry per 80,000 seed. Seeds were circulated in treater bowl for 15-25 seconds to uniformly coat the seed. Treated seed were air dried prior to planting.

TABLE 1
Fungicide seed treatment components used in corn hyperspectral experiments

| | | | | | Rate |
|---|---|---|---|---|---|
| | | | | | (mg |
| Protocol | n | Treatment | Component | Type | ai/seed) |
| Fungicide, | 1 | Wild-type (not treated) | Not treated | | |
| Fungicide + | 2 | Ethaboxam | Ethaboxam | Fungicide | 0.0141 |
| polymer | | | Red Color | Colorant | 0.0552 |
| | 3 | Picoxystrobin 0.005 | Picoxystrobin | Fungicide | 0.005 |
| | | | Red Color | Colorant | 0.0552 |
| | 4 | Picoxystrobin 0.010 | Picoxystrobin | Fungicide | 0.010 |
| | | | Red Color | Colorant | 0.0552 |
| | 5 | Picoxystrobin 0.005 + | Picoxystrobin | Fungicide | 0.005 |
| | | Polymer | Polymer | Polymer | 0.167 |
| | | | Red Color | Colorant | 0.0552 |
| | 6 | Picoxystrobin 0.01 + | Picoxystrobin | Fungicide | 0.01 |
| | | Polymer | Polymer | Polymer | 0.167 |
| | | | Red Color | Colorant | 0.0552 |

TABLE 2
Mahalanobis distance (log transformation) of corn hybrids P9998CYXR and P135YHRCE treated
with fungicide or fungicide + polymer seed treatments and wild-type (not treated seed).

| | | Seed Treatment B | | | | | | |
|---|---|---|---|---|---|---|---|---|
| | | | | | | Picoxy- | | Picoxy- |
| | | Wild-type | | Etha- | Picoxy- | strobin | Picoxy- | strobin |
| | | (not | Etha- | boxam + | strobin | 0.005 mg + | strobin | 0.01 mg + |
| Hybrid | Seed Treatment A | treated) | boxam | polymer | 0.005 mg | polymer | 0.01 mg | polymer |
| P9998CYXR | Wild-type (not treated) | 0 | | | | | | |
| P9998CYXR | Ethaboxam | 4.9801 | 0 | | | | | |
| P9998CYXR | Ethaboxam + polymer | 5.0386 | 0.3829 | 0 | | | | |
| P9998CYXR | Picoxystrobin 0.005 mg | 5.1965 | 3.7174 | 3.7545 | 0 | | | |
| P9998CYXR | Picoxystrobin 0.005 mg + polymer | 4.8022 | 3.5847 | 3.6667 | 0.8251 | 0 | | |
| P9998CYXR | Picoxystrobin 0.01 mg | 3.0864 | 4.0442 | 4.5257 | 3.8605 | 3.8264 | 0 | |
| P9998CYXR | Picoxystrobin 0.01 mg + polymer | 3.3668 | 3.7064 | 3.7595 | 3.8741 | 3.6688 | 1.5994 | 0 |
| P1395YHR | Wild-type (not treated) | 0 | | | | | | |
| P1395YHR | Ethaboxam | 4.4394 | 0 | | | | | |
| P1395YHR | Ethaboxam + polymer | 4.9356 | 1.1992 | 0 | | | | |
| P1395YHR | Picoxystrobin 0.005 mg | 4.7608 | 3.3788 | 3.4799 | 0 | | | |
| P1395YHR | Picoxystrobin 0.005 mg + polymer | 5.044 | 3.7698 | 3.5063 | 1.1395 | 0 | | |
| P1395YHR | Picoxystrobin 0.01 mg | 5.6625 | 3.7522 | 3.8479 | 4.262 | 4.3008 | 0 | |
| P1395YHR | Picoxystrobin 0.01 mg + polymer | 5.5571 | 3.8223 | 3.6914 | 4.151 | 4.1854 | 0.7976 | 0 |

Corn FST experiments were planted in the Corteva Agriscience (Johnston, IA) greenhouse facilities, with two different planting dates of 10 replicates per seed treatment in each planting date. Planting date 1 was 5 Aug. 2020 and planting date 2 was 25 Jan. 2021. Seed was planted at a standard planting depth of 20 to 25 mm in a standard germination mix comprising of 76.49% peat, 16.10% peat, 7.38% vermiculite, 5 lbs/yd3 lime, 3 lbs/yd3 starter fertilizer, and 0.5 lbs/yd3 wetting agent. The individual seeds were placed in flats of 32 plants each in an 8×4 configuration resulting in a 212 plants/m2 density.

The plants were grown in a glass greenhouse with of a day length of 16 hours and growth conditions of daytime temperature at 22.22 °C, and nighttime temperature at 19.44 °C. Plants were imaged at first visual sight of the plumule using a Hyperspectral Visible and Near InfraRed camera and a short-wave infrared camera. The cameras capture reflectance between 400 nm and 2500 nm. Plants grown under normal conditions were imaged daily from 5 days after emergence (DAE) until approximately 9 DAE (V0 to V3 growth stage). Data were processed using proprietary software into average reflectance spectra of individual plants and physiologically relevant features. These data were standardized and processed using R (R is a statistical software package and coding language). Custom algorithms were developed to adjust data and remove variability. Data were processed using a modified version of Mahalanobis distance to determine differences between treatments. Resulting distance matrices were log-transformed, with values over 3 considered significantly different.

### Results and Discussion:

The Mahalanobis distance is a measure of the distance between a datapoint and the distribution of data; quantifying the number of standard deviations the datapoint is away from the mean of the distribution. The larger the value, the further away the seed treatment is from the wild-type not treated control. For the corn fungicide seed treatments across both hybrids tested, the ethaboxam, ethaboxam+polymer, picoxystrobin (0.005 mg, 0.01 mg), and picoxystrobin (0.005 mg, 0.01 mg)+polymer treatments were the most different compared to the wild-type control, with log transformed standard deviations of 3 or higher (Table 2). The ethaboxam treatments and picoxystrobin 0.005 mg treatments were most different from control, with values greater than 4.4 (Table 2). In each case of a paired comparison of a single fungicide with its corresponding fungicide+polymer treatment, there were no significant differences detected across both hybrids. Ethaboxam compared to ethaboxam+polymer, picoxystrobin 0.005 mg compared to picoxystrobin 0.005 mg+polymer, and picoxystrobin 0.01 mg compared to picoxystrobin 0.01 mg+polymer were all 1.6 standard deviations or less (Table 2). Morphological characteristics derived from hyperspectral imaging of total pixel area, green index, yellow index, and brown index were evaluated for the different corn fungicide seed treatments (Table 3). The pixel area was a good indicator of plant size (plant vigor and growth), while the color indices (green, yellow, or brown) were identified to be a potential indicator of plant health. Across the plant ages for both hybrids, the average total pixel area trait had the most consistent magnitude of differences evident from 5 to 9 DAE for the FST concepts evaluated (Table 3).

TABLE 3
Hyperspectral differences¹ of corn hybrids P9998CYXR or P1395YHR treated with fungicide
or fungicide + polymer seed treatments compared to WT (wild-type not treated control)

| | | P9998CYXR | | | | P1395YHR | | | |
|---|---|---|---|---|---|---|---|---|---|
| | | Avg | Avg | Avg | Avg | Avg | Avg | Avg | Avg |
| Plant | | Pixel | Green | Yellow | Brown | Pixel | Green | Yellow | Brown |
| age | Seed Treatment Comparison | Area | Index | Index | Index | Area | Index | Index | Index |
| 5 | Ethaboxam - WT | 1.8 | 3.4 | −0.3 | −2.1 | 2.4 | −7.2 | 1.4 | −4.8 |
| 5 | Ethaboxam polymer - WT | 10.1 | 4.4 | −1.5 | −2.9 | 10.8 | −6.7 | 0 | −1.5 |
| 5 | Picoxystrobin 0.005 mg - WT | 0.4 | 3.2 | −3.3 | −0.3 | 1.7 | −13.3 | −4.8 | 0.6 |
| 5 | Picoxystrobin 0.005 mg | 5.9 | 3.4 | −1.8 | −1.2 | 9.2 | −13.1 | −4.4 | 2.4 |
| | polymer - WT | | | | | | | | |
| 5 | Picoxystrobin 0.01 mg - WT | −1.6 | −2.8 | 3.2 | 5.4 | −2.2 | −17.6 | 2 | 17.2 |
| 5 | Picoxystrobin 0.01 mg | 4.3 | −1 | 2.1 | 3.1 | 4.5 | −16 | 1.2 | 14.3 |
| | polymer - WT | | | | | | | | |
| 6 | Ethaboxam - WT | 5.6 | 0.6 | −3.4 | −5.4 | 1.9 | −1 | 1.3 | −6.5 |
| 6 | Ethaboxam polymer - WT | 8.7 | 1.1 | −3.3 | −6 | 12.4 | 0.3 | 3.2 | −7 |
| 6 | Picoxystrobin 0.005 mg - WT | 2.7 | 0.8 | −6.1 | 0.2 | 2.6 | −0.3 | −2 | −7.8 |
| 6 | Picoxystrobin 0.005 mg | 5.9 | 2.2 | −5.7 | −1.8 | 9.4 | 0 | −1.4 | −7.6 |
| | polymer - WT | | | | | | | | |
| 6 | Picoxystrobin 0.01 mg - WT | −0.2 | −2.5 | −7.9 | 4 | −4.8 | −1.7 | −4.6 | 5.4 |
| 6 | Picoxystrobin 0.01 mg | 5.4 | −1.5 | −6.5 | 1.5 | 1.2 | −1.4 | −4.1 | 4.1 |
| | polymer - WT | | | | | | | | |
| 7 | Ethaboxam - WT | 5.6 | 9.4 | −4.7 | −2.3 | 1 | 6.8 | 2.5 | −15.5 |
| 7 | Ethaboxam polymer - WT | 7.4 | 9.3 | −4.5 | −2.3 | 7.8 | 7.4 | 3.4 | −16.5 |
| 7 | Picoxystrobin 0.005 mg - WT | 2.5 | 2.6 | −9.9 | 4.7 | 1.3 | 6.4 | 0.7 | −14.2 |
| 7 | Picoxystrobin 0.005 mg | 4.9 | 2.6 | −9.3 | 4.4 | 7.1 | 6.4 | 0.7 | −14.1 |
| | polymer - WT | | | | | | | | |
| 7 | Picoxystrobin 0.01 mg - WT | −0.3 | 1.4 | −7.8 | 6.1 | −4.8 | 0.8 | −3.1 | −2.7 |
| 7 | Picoxystrobin 0.01 mg | 4 | 3.3 | −6.3 | 4.3 | 0.6 | 0.8 | −2.7 | −3.3 |
| | polymer - WT | | | | | | | | |
| 8 | Ethaboxam - WT | 8.9 | 581.1 | −22.3 | −125.1 | −0.1 | −59.9 | −181.2 | 304.5 |
| 8 | Ethaboxam polymer - WT | 11.1 | 566.2 | −19.2 | −126.2 | 1.2 | −57.9 | −186.6 | 312.7 |
| 8 | Picoxystrobin 0.005 mg - WT | 6.9 | 402.7 | −10.6 | −131 | 0.4 | 45.7 | −39.8 | 58.4 |
| 8 | Picoxystrobin 0.005 mg | 9.9 | 416 | −3.2 | −145.1 | 0.8 | 43.8 | −34.6 | 55.6 |
| | polymer - WT | | | | | | | | |
| 8 | Picoxystrobin 0.01 mg - WT | 3.9 | 250.6 | 76.9 | 10.2 | −1.2 | −18.3 | −249.5 | 362.7 |
| 8 | Picoxystrobin 0.01 mg | 9.4 | 269.2 | 77.9 | −6.2 | −0.3 | −20.9 | −243.8 | 365.7 |
| | polymer - WT | | | | | | | | |
| 9 | Ethaboxam - WT | 5.4 | 35.1 | −120.6 | −2 | −2 | −21 | −85.5 | 47.8 |
| 9 | Ethaboxam polymer - WT | 5.3 | 36.4 | −132.2 | −2.3 | 1.3 | −24 | −71.9 | 58.6 |
| 9 | Picoxystrobin 0.005 mg - WT | 2.7 | 0.5 | −49.5 | −4.1 | −1.4 | 22.4 | 24.4 | 7.9 |
| 9 | Picoxystrobin 0.005 mg | 4.4 | 0.2 | −68 | −3.1 | 1.7 | 21.8 | 11.5 | 5.7 |
| | polymer - WT | | | | | | | | |
| 9 | Picoxystrobin 0.01 mg - WT | 1.7 | 1.8 | 99.3 | 6.9 | −0.7 | −15.8 | −120.6 | 85.7 |
| 9 | Picoxystrobin 0.01 mg | 5.6 | 3.2 | 100.6 | −0.4 | 0.2 | −16.1 | −112.9 | 89.2 |
| | polymer - WT | | | | | | | | |

¹Positive values indicate better performance compared to WT (wild-type not treated control), negative values indicate worse performance compared to wild-type (not treated)

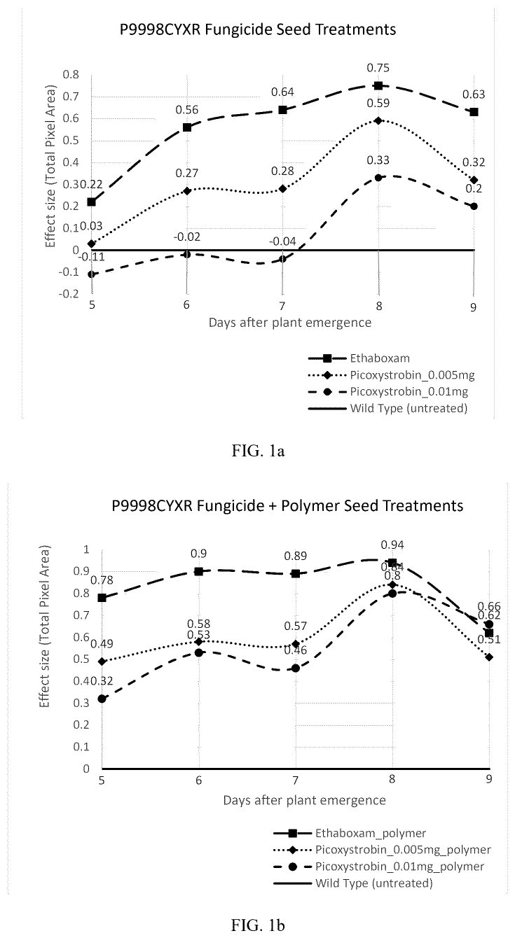

In graphing the difference in average pixel area over time for corn hybrid P9998CYXR treated with fungicide or fungicide+polymer seed treatments, the ethoboxam and ethaboxam+polymer treatments had the highest positive pixel area compared to the wild-type control across all plant growth stages (FIG. 1). There was a rate difference detected, whereby the picoxystrobin 0.005 mg treatment had higher total pixel area compared to the picoxystrobin 0.01 mg treatment for all plant ages (FIG. 1). For each FST on corn hybrid P9998CYXR, the addition of a polymer enhanced the overall total pixel area when compared to the respective FST alone at each plant growth stage (FIG. 1).

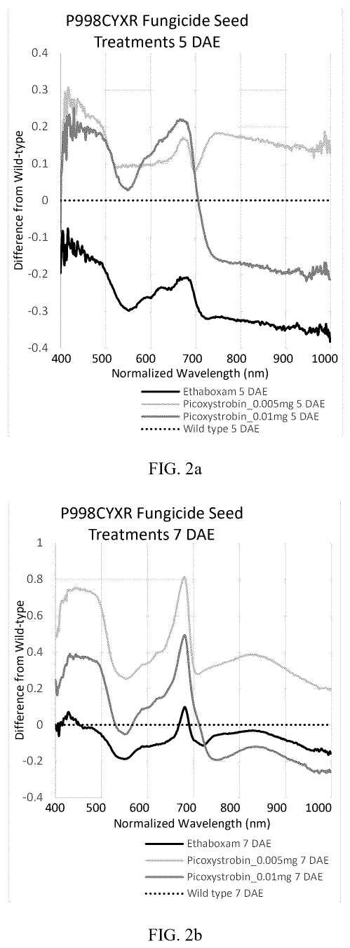

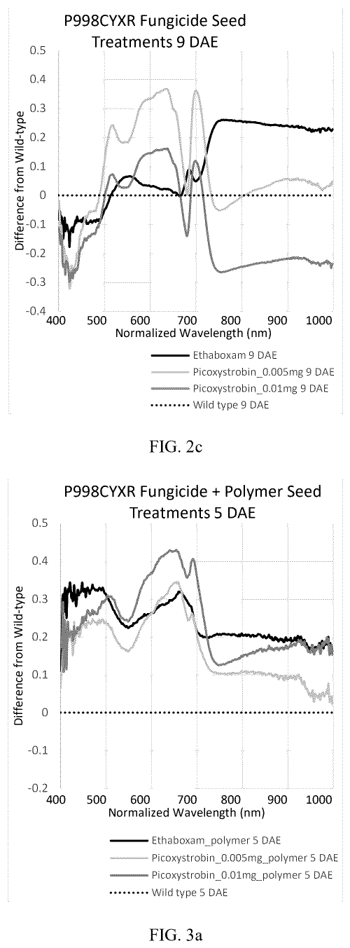

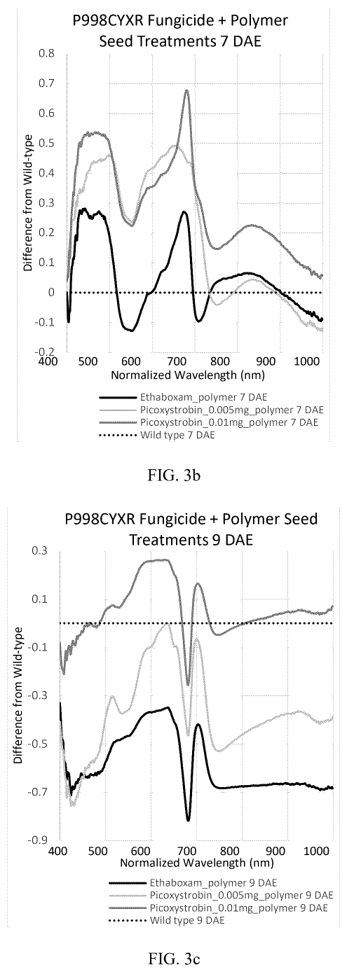

Analyzing the hyperspectal reflectance of individual wavelengths for corn hybrid P999CYXR treated with fungicides, the ethaboxam treatment and picoxystrobin 0.01 mg treatment had similar patterns of reflectance at 5 and 7 DAE, though ethaboxam was consistently lower than WT on both dates (FIG. 2). At 9 DAE, the ethaboxam treatment shows a unique reflectance pattern when compared to both picoxystrobin treatments (FIG. 2). The lower rate of picoxystrobin (0.005 mg) consistently had higher reflectance values, despite the pattern being unique beyond 700 nm at 5 DAE (FIG. 2). With the addition of the polymer to each of the above treatments, ethaboxam still results in lower reflectance values at the later stages, 7 and 9 DAE (FIG. 3). The reflectance pattern of all three treatments is more consistent across all three time points than without polymer, despite some fluctuations at 5 and 7 DAE. Together, these differential patterns between treatments indicate unique signal and resolving power using HS systems to differentiate seed treatment up to 9 DAE (FIGS. 2 and 3).

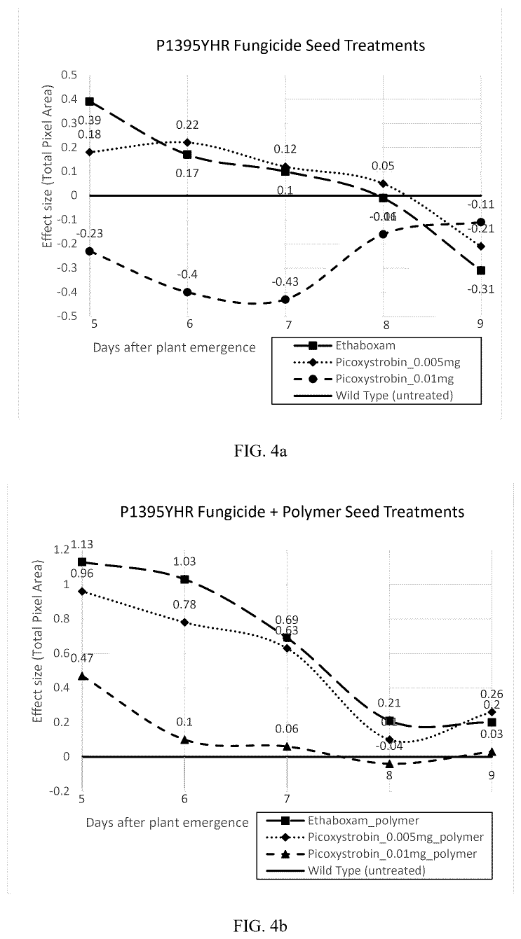

For corn hybrid P1395YHRCE, the ethaboxam treatment had higher pixel area compared to the wild-type control from 5 days to 8 days after emergence (FIG. 4). The ethaboxam+polymer treatment had higher pixel area compared to control across all the growth stages evaluated (FIG. 4). The picoxystrobin 0.005 mg had higher pixel area when compared to picoxystrobin 0.10 mg until 8 days after emergence (FIG. 4). The picoxystrobin 0.005 mg+polymer treatment had higher pixel area compared to picoxystrobin 0.01 mg+polymer across all the growth stages tested. As was evident with corn hybrid P999CYXR, each fungicide+polymer treatment on corn hybrid P1395YHRCE had higher values of total pixel area when compared to the respective non polymer treatment at each respective growth stage (FIGS. 1, 4)

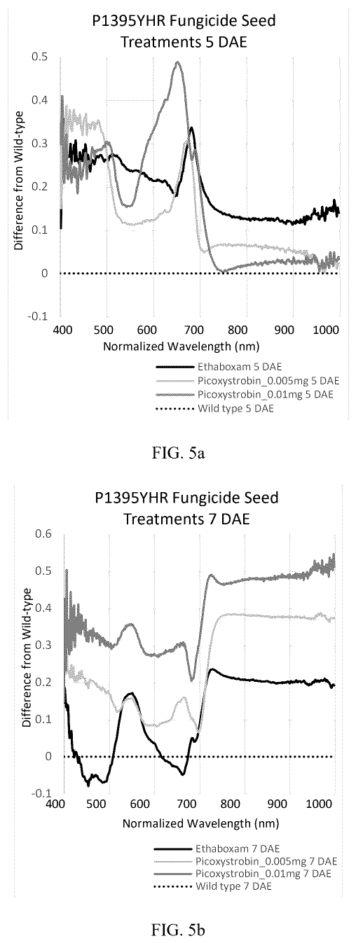

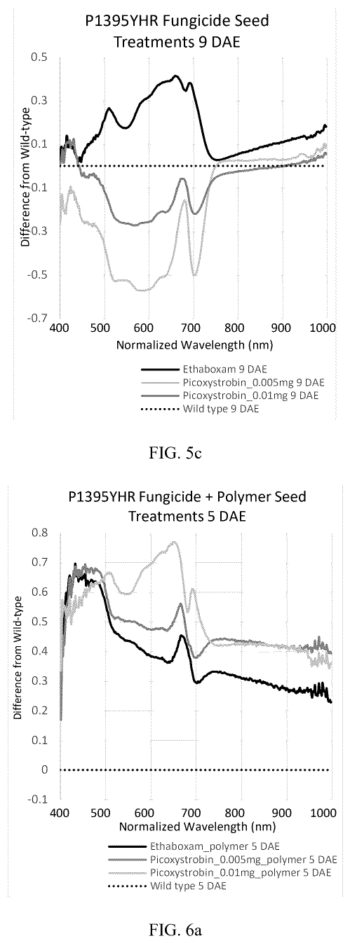

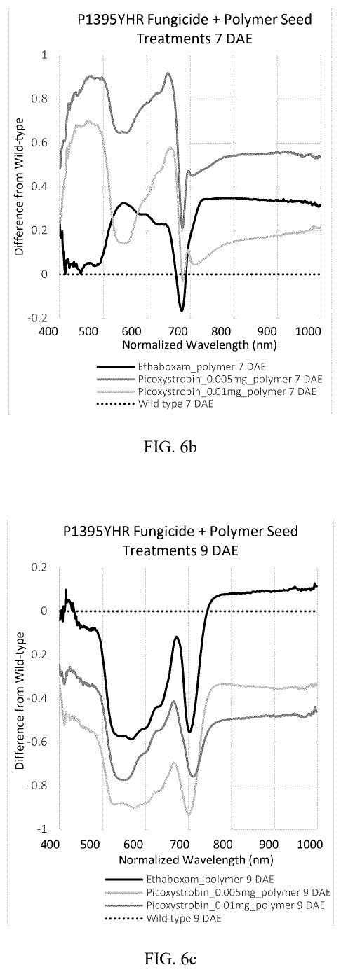

The ethaboxam seed treatment on corn hybrid P1395HRCE in general had positive differences from wild-type across all hyperspectral wavelengths from 5 to 9 DAE (FIG. 5). Spectral reflectance patterns were similar at 7 DAE for all treatments but differed at younger and older stages. At 5 DAE, picoxystrobin at 0.01 mg has a blue shift in reflectance peak, while at 9 DAE, ethaboxam has inverse reflectance as compared to both picoxystrobin treatments (FIG. 5). Similar to corn hybrid P9998, the addition of the polymer to the treatments appears to stabilize the reflectance fingerprint of each treatment, resulting in more uniform, non-overlapping patterns at 9 DAE (FIG. 6).

The data shown above indicate a clear ability to differentiate corn fungicide seed treatments from one another using hyperspectral imaging on seedlings prior to growth stage V4. This differentiation can be seen in morphological data (pixel area, green index, yellow index, brown index) and hyperspectral reflectance signature pattern at different timepoints. Hyperspectral imaging applied in this way is a novel application of the technology, elucidating and quantifying differences between corn fungicide seed treatments and different fungicide treatment rates.

### Example 2. Corn Insecticide Seed Treatments

### Materials and Methods:

Corn commercial hybrids P9998CYXR and P1395YHR were selected based upon unique genetic background and adaptation to different regions of the United States. Corn seed were treated with different insecticide seed treatments (IST) (Table 4). Corn hybrid seed were treated as water-based slurries using a Hege 11 laboratory seed treater (Wintersteiger AG, Ried im Innkreis, Austria). Slurries were created by mixing the treatment components in a sufficient volume of tap water to ensure uniform seed coverage. Prepared slurries were pipetted onto the spinning disk of a Hege 11 seed treater in application volumes of 90-130 mL slurry per 80,000 seed. Seeds were circulated in treater bowl for 15-25 seconds to uniformly coat the seed. Treated seed were air dried prior to planting.

Corn IST experiments were planted in the Corteva Agriscience (Johnston, IA) greenhouse facilities, with two different planting dates of 10 replicates per seed treatment in each planting date. Planting date 1 was 5 Aug. 2020 and planting date 2 was 25 Jan. 2021. Seed was planted at a standard planting depth of 20 to 25 mm in a standard germination mix comprising of 76.49% peat, 16.10% peat, 7.38% vermiculite, 5 lbs/yd³ lime, 3 lbs/yd³ starter fertilizer, and 0.5 lbs/yd³ wetting agent. The individual seeds were placed in flats of 32 plants each in an 8×4 configuration resulting in a 212 plants/m² density.

The plants were grown in a glass greenhouse with of a day length of 16 hours and growth conditions of daytime temperature at 22.22 °C, and nighttime temperature at 19.44 °C. Plants were imaged at first visual sight of the plumule using a Hyperspectral Visible and Near InfraRed camera and a short-wave infrared camera. The cameras capture reflectance between 400 nm and 2500 nm. Plants grown under normal conditions were imaged daily from 5 days after emergence (DAE) until approximately 9 DAE (V0 to V3 growth stage). Data were processed using proprietary software into average reflectance spectra of individual plants and physiologically relevant features. These data were standardized and processed using R. Custom algorithms were developed to adjust data and remove variability. Data were processed using a modified version of Mahalanobis distance to determine differences between treatments. Resulting distance matrices were log-transformed, with values over 3 considered significantly different.

### Example 2. Results and Discussion

The Mahalanobis distance is a measure of the distance between a datapoint and the distribution of data; quantifying the number of standard deviations the datapoint is away from the mean of the distribution. The larger the value, the further away the seed treatment is from the wild-type not treated control. Comparing the log transformation of Mahalanobis distance for corn IST indicated each insecticide or insecticide+polymer treatment was significantly different compared to the wild-type control, with values greater than 3.6 (Table 5). In general, for each hybrid the chlorantraniliprole treatments had higher values compared to the cyantraniliprole treatments, indicating chlorantraniliprole was more different compared to wild-type control (Table 5). For both hybrids, the addition of the polymer was not significant compared to the respective insecticide only treatment, as the values were less than 1.4 standard deviations in each paired comparison (Table 5).

Morphological characteristics derived from hyperspectral imaging of total pixel area, green index, yellow index, and brown index were evaluated for the different corn IST (Table 6). Across the different plant ages, the average total pixel area trait had the most consistent magnitude of differences evident for all IST treatments and both hybrids tested from 5 to 9 DAE (Table 6). The color indices (green, yellow, or brown) did not have a magnitude of differences evident until a large difference was observed at 8 to 9 DAE (Table 6).

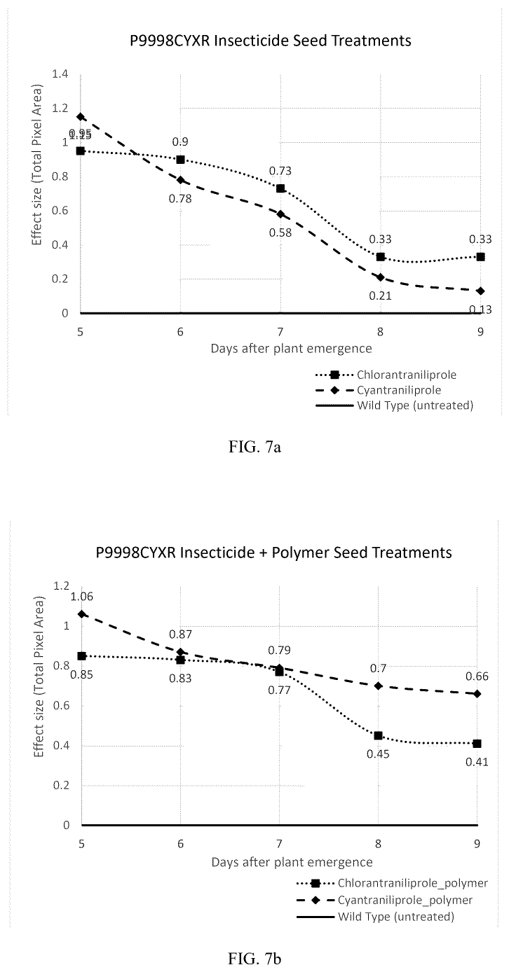

In graphing the pixel area standard deviation from control for corn hybrid P9998CYXR, both the chlorantraniliprole and cyantraniliprole seed treatments (with and without a polymer) had more pixel area compared to the wild-type control from 5 to 9 DAE (FIG. 7). The difference in values was generally highest at 5 days after emergence, with decreasing differences observed until 9 days after plant emergence (FIG. 7). The addition of a polymer resulted in higher pixel area compared to the individual IST by itself for both chlorantraniliprole and cyantraniliprole (FIG. 7).

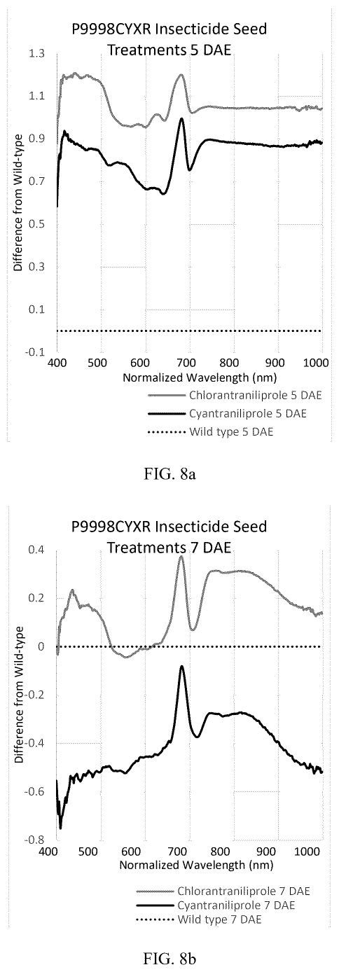

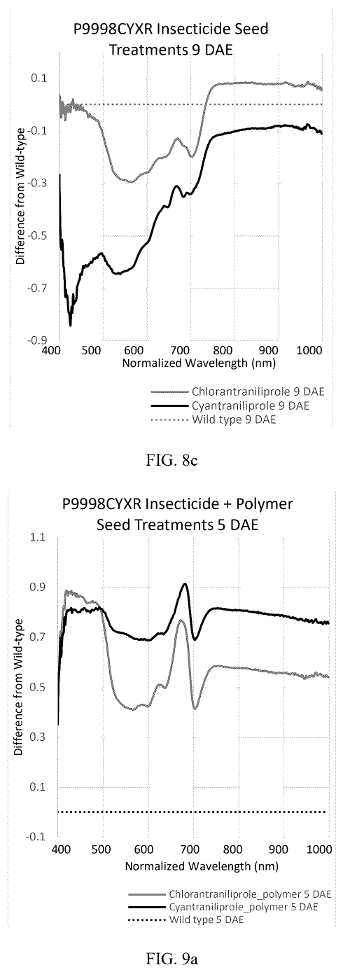

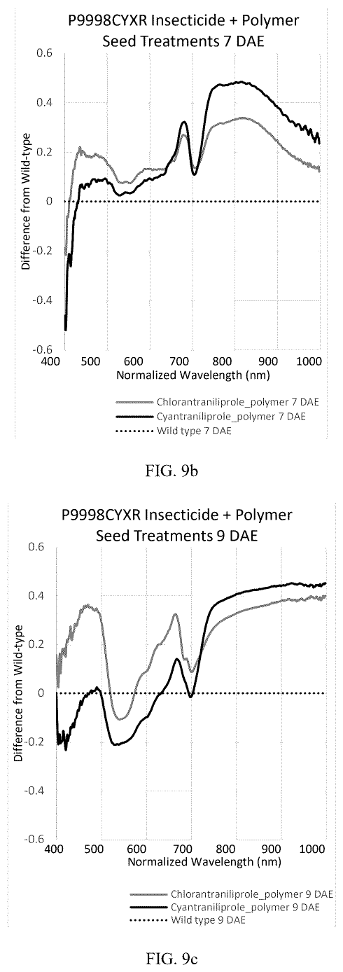

Examining the hyperspectral wavelengths over time for corn hybrid P9998CYXR, both IST had positive differences from wild-type at 5 DAE (FIG. 8). Across the different growth stages and at all wavelengths, the chlorantraniliprole treatment had higher values compared to the cyantraniliprole treatment (FIG. 8). The spectral reflectance fingerprints followed similar trends across all three ages for both IST (FIG. 8). The addition of the polymer appears to reduce the reflectance dynamics of the chlorantraniliprole treatment on hybrid P9998CYXR, while retaining a similar pattern for the cyantraniliprole treatment (FIG. 9).

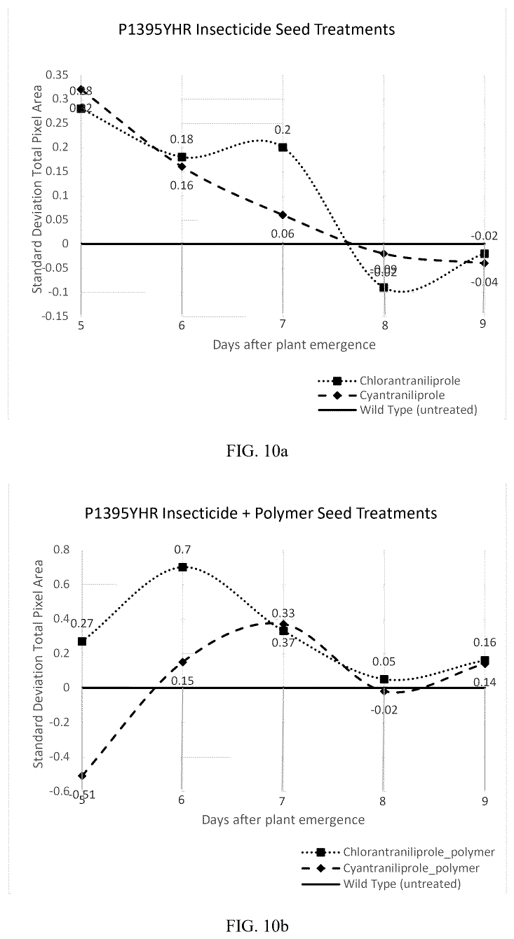

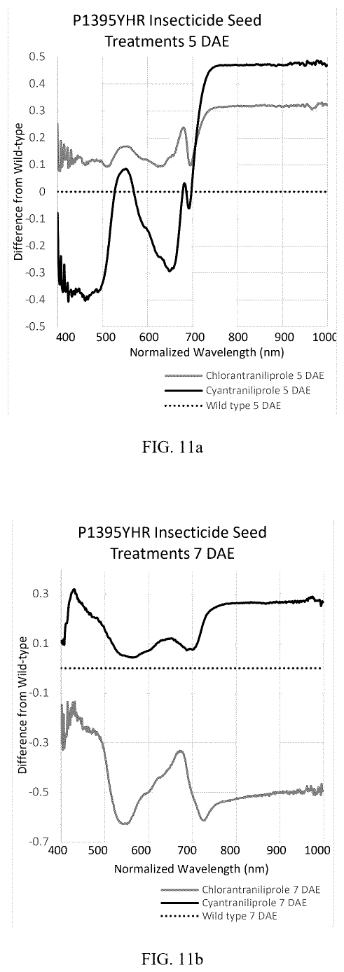

For corn hybrid P1395YHR, average pixel area difference compared to control was variable for the two insecticide seed treatments. Chlorantraniliprole and cyantraniliprole treatments had significantly more pixel area difference compared to control from 5 to 7 days after emergence (FIG. 10). Compared to the chlorantraniliprole only treatment, the addition of a polymer generally increased the pixel area for all growth stages (FIG. 10). The addition of a polymer to the cyantraniliprole treatment did not have a consistent effect on pixel area compared to cyantraniliprole by itself (FIG. 10).

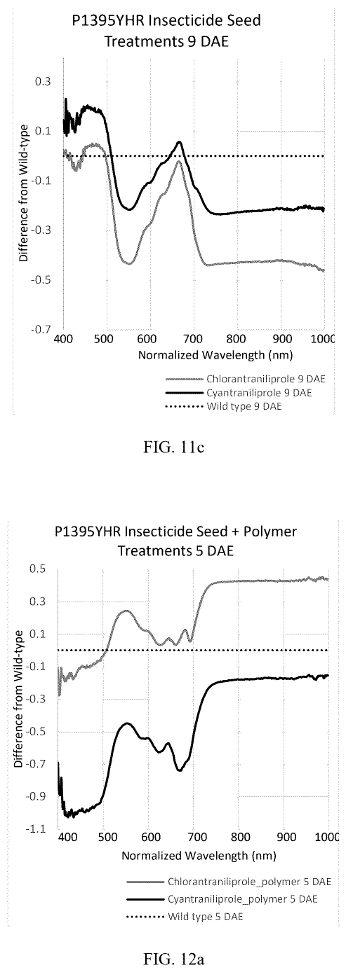

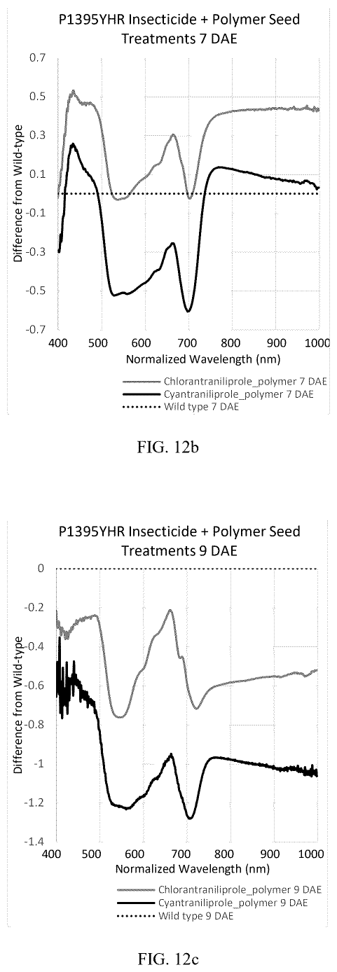

Examining the hyperspectral wavelength differences for corn hybrid P1395YHR, at 5 DAE the cyantraniliprole treatment had higher values above 700 nm compared to chlorantraniliprole (FIG. 11). At both 7 DAE and 9 DAE, the cyantraniliprole treatment had higher values compared to the chlorantraniliprole treatment for all frequencies (FIG. 11). When polymers are added to the insecticides for hybrid P1395YHR, the chlorantraniliprole+polymer treatment had higher values compared to the cyantraniliprole+polymer treatment for all hyperspectral frequencies at all time points graphed (FIG. 12). The reflectance curves for chlorantraniliprole+polymer treatment and the cyantraniliprole+polymer treatment followed similar patterns across time (FIG. 12).

The data indicate a clear ability to differentiate corn insecticide seed treatments from one another using hyperspectral imaging on seedlings. This differentiation can be seen in morphological data (pixel area, green index, yellow index, brown index) and unique hyperspectral reflectance signature pattern at different timepoints. Hyperspectral imaging applied in this way is a novel application of the technology, enabling the elucidation and quantification of differences between corn insecticide seed treatments.

TABLE 4
Insecticide seed treatment components used in corn hyperspectral experiments

| | | | | | Rate |
|---|---|---|---|---|---|
| | | | | | (mg |
| Protocol | n | Treatment | Component | Type | ai/seed) |
| Insecticide, | 1 | Wild-type (not treated) | Not treated | | |
| Insecticide + | 2 | Chlorantraniliprole 0.25 | Chlorantraniliprole | Insecticide | 0.25 |
| polymer | | | Red Color | Colorant | 0.0552 |
| | 3 | Cyantraniliprole 0.75 | Cyantraniliprole | Insecticide | 0.75 |
| | | | Red Color | Colorant | 0.0552 |
| | 4 | Chlorantraniliprole 0.25 + | Chlorantraniliprole | Insecticide | 0.25 |
| | | Polymer | Polymer | Polymer | 0.167 |
| | | | Red Color | Colorant | 0.0552 |
| | 5 | Cyantraniliprole 0.75 + | Cyantraniliprole | Insecticide | 0.75 |
| | | Polymer | Polymer | Polymer | 0.167 |
| | | | Red Color | Colorant | 0.0552 |

TABLE 5
Mahalanobis distance (log transformation) of corn hybrids P9998CYXR and P135YHRCE treated
with insecticide or insecticide + polymer seed treatments and wild-type (not treated seed)

| | | Seed Treatment B | | | | |
|---|---|---|---|---|---|---|
| | | | | Chloran- | | Cyantran- |
| | | Wild-type | Chloran- | traniliprole + | Cyantran- | iliprole + |
| Hybrid | Seed Treatment A | (not treated) | traniliprole | polymer | iliprole | polymer |
| P9998CYXR | Wild-type (not treated) | 0 | | | | |
| P9998CYXR | Chlorantraniliprole | 5.4795 | 0 | | | |
| P9998CYXR | Chlorantraniliprole + polymer | 5.2296 | 0.1014 | 0 | | |
| P9998CYXR | Cyantraniliprole | 5.1798 | 4.4596 | 3.9493 | 0 | |
| P9998CYXR | Cyantraniliprole + polymer | 5.0318 | 3.8143 | 3.3183 | 0.9135 | 0 |
| P1395YHR | Wild-type (not treated) | 0 | | | | |
| P1395YHR | Chlorantraniliprole | 4.0798 | 0 | | | |
| P1395YHR | Chlorantraniliprole + polymer | 4.2752 | 0.0261 | 0 | | |
| P1395YHR | Cyantraniliprole | 4.4467 | 2.7241 | 2.8577 | 0 | |
| P1395YHR | Cyantraniliprole + polymer | 3.5725 | 2.749 | 2.7561 | 1.3987 | 0 |

TABLE 6
Hyperspectral differences¹ of corn hybrids P9998CYXR or P1395YHR treated with insecticide
or insecticide + polymer seed treatments compared to WT (wild-type not treated control)

| | | P9998CYXR | | | | P1395YHR | | | |
|---|---|---|---|---|---|---|---|---|---|
| | | Avg | Avg | Avg | Avg | Avg | Avg | Avg | Avg |
| Plant | Seed Treatment | Pixel | Green | Yellow | Brown | Pixel | Green | Yellow | Brown |
| age | Comparison | Area | Index | Index | Index | Area | Index | Index | Index |
| 5 | Chlorantraniliprole - WT | 24.3 | 74 | −31.3 | −28 | 1.5 | −232.9 | −5.5 | 253.2 |
| 5 | Chlorantraniliprole polymer - WT | 21.9 | 73 | −30.4 | −27.6 | 1.5 | −232.2 | −6.1 | 250.9 |
| 5 | Cyantraniliprole - WT | 29.4 | 10.9 | −68.3 | 13.1 | 1.7 | 72.5 | 51.6 | −354.1 |
| 5 | Cyantraniliprole polymer - WT | 27.1 | 13.7 | −80.4 | 14.3 | −2.8 | 83.9 | 33.9 | −325 |
| 6 | Chlorantraniliprole - WT | 12.7 | 2.4 | 0.4 | −5.2 | 1.1 | −1.2 | −4.5 | −1 |
| 6 | Chlorantraniliprole polymer - WT | 11.7 | 1.7 | 0.3 | −4.9 | 4.1 | −0.4 | −4.2 | −1.9 |
| 6 | Cyantraniliprole - WT | 11.1 | 2.4 | −3.4 | −5 | 1 | 2.7 | −1.7 | −2.7 |
| 6 | Cyantraniliprole polymer - WT | 12.2 | 2 | −3.8 | −3.8 | 0.9 | 4.1 | −2.6 | −4 |
| 7 | Chlorantraniliprole - WT | 9.9 | 3.8 | 5.7 | −4.2 | 1.5 | −0.5 | −16.3 | 7.1 |
| 7 | Chlorantraniliprole polymer - WT | 10.3 | 4.3 | 6.5 | −4.5 | 2.5 | 0.3 | −15.1 | 5.1 |
| 7 | Cyantraniliprole - WT | 7.8 | 11.1 | 4 | −10.3 | 0.5 | 1.4 | 12.8 | 1.1 |
| 7 | Cyantraniliprole polymer - WT | 10.6 | 10.8 | 5.4 | −11.2 | 2.8 | 2 | 10.6 | −0.9 |
| 8 | Chlorantraniliprole - WT | 4.3 | −12.8 | −87.7 | 46.9 | −0.6 | −14.1 | −109.8 | 56.6 |
| 8 | Chlorantraniliprole polymer - WT | 5.8 | −8.3 | −87.1 | 32.4 | 0.4 | −14.1 | −107.1 | 56.3 |
| 8 | Cyantraniliprole - WT | 2.7 | 37.9 | 109.1 | −143.8 | −0.1 | −13.1 | −39 | 58.5 |
| 8 | Cyantraniliprole polymer - WT | 9 | 36.2 | 74.8 | −133.3 | −0.1 | −14.2 | −46.1 | 61.6 |
| 9 | Chlorantraniliprole - WT | 4.2 | 247.5 | −8.9 | −141.1 | −0.2 | 19.7 | −771.2 | 108 |
| 9 | Chlorantraniliprole polymer - WT | 5.3 | 245.8 | −13.9 | −137.9 | 1.2 | 15.1 | −745.4 | 95.6 |
| 9 | Cyantraniliprole - WT | 1.6 | 512.1 | 560.5 | −361.6 | −0.3 | 10.2 | −174.7 | 150.6 |
| 9 | Cyantraniliprole polymer - WT | 8.6 | 462.8 | 549.1 | −353.8 | 1 | 10.3 | −207.6 | 149.6 |

¹Positive values indicate better performance compared to WT (wild-type not treated control), negative values indicate worse performance compared to wild-type (not treated)

### Example 3. Corn Biological Nematicide Seed Treatments

### Materials and Methods:

Corn commercial hybrids P9998CYXR and P1395YHR were selected based upon unique genetic background and adaptation to different regions of the United States. Corn seed were treated with different biological nematicide seed treatments (Table 7). Corn hybrid seed were treated as water-based slurries using a Hege 11 laboratory seed treater (Wintersteiger AG, Ried im Innkreis, Austria). Slurries were created by mixing the treatment components in a sufficient volume of tap water to ensure uniform seed coverage. Prepared slurries were pipetted onto the spinning disk of a Hege 11 seed treater in application volumes of 90-130 mL slurry per 80,000 seed. Seeds were circulated in treater bowl for 15-25 seconds to uniformly coat the seed. Treated seed were air dried prior to planting.

Corn biological nematicide seed treatment experiments were planted in the Corteva Agriscience (Johnston, IA) greenhouse facilities, with two different planting dates of 10 replicates per seed treatment in each planting date. Planting date 1 was 5 Aug. 2020 and planting date 2 was 25 Jan. 2021. Seed was planted at a standard planting depth of 20 to 25 mm in a standard germination mix comprising of 76.49% peat, 16.10% peat, 7.38% vermiculite, 5 lbs/yd³ lime, 3 lbs/yd³ starter fertilizer, and 0.5 lbs/yd³ wetting agent. The individual seeds were placed in flats of 32 plants each in an 8×4 configuration resulting in a 212 plants/m² density.

The plants were grown in a glass greenhouse with of a day length of 16 hours and growth conditions of daytime temperature at 22.22 °C, and nighttime temperature at 19.44 °C. Plants were imaged at first visual sight of the plumule using a Hyperspectral Visible and Near InfraRed camera and a short-wave infrared camera. The cameras capture reflectance between 400 nm and 2500 nm. Plants grown under normal conditions were imaged daily from 5 days after emergence (DAE) until approximately 9 DAE (V0 to V3 growth stage). Data were processed using proprietary software into average reflectance spectra of individual plants and physiologically relevant features. These data were standardized and processed using R. Custom algorithms were developed to adjust data and remove variability. Data were processed using a modified version of Mahalanobis distance to determine differences between treatments. Resulting distance matrices were log-transformed, with values over 3 considered significantly different.

### Results and Discussion:

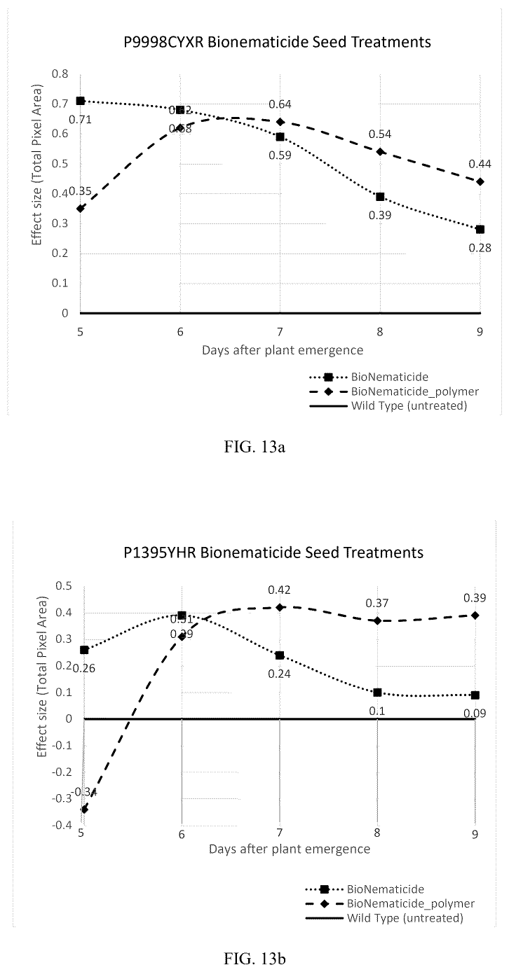

The Mahalanobis distance is a measure of the distance between a datapoint and the distribution of data; quantifying the number of standard deviations the datapoint is away from the mean of the distribution. The larger the value, the further away the seed treatment is from the wild-type not treated control. The log transformation of Mahalanobis distance was significantly different for the biological nematicide treatments (with and without a polymer) on P9998CYXR, with values over 4.1 (Table 8). The biological nematicide treatments (with or without a polymer) were not significantly different compared to the wild-type control for the P1395YHR hybrid (Table 8). The biological nematicide treatment itself was not significantly different compared to the biological nematicide+polymer treatment for both hybrids, with values less than 1 (Table 8). The average standard deviation difference for pixel area data were graphed for seed treatments that had a biological nematicide or biological nematicide+polymer. For hybrid P9998CYXR, the biological nematicide and biological nematicide+polymer treatments were higher for pixel area compared to the wild-type control across all plant stages (FIG. 13). For the P1395YHR hybrid, the biological nematicide treatment had higher pixel area compared to control for all plant stages, while the biological nematicide+polymer treatment was better compared to control at 6 to 9 DAE (FIG. 13).

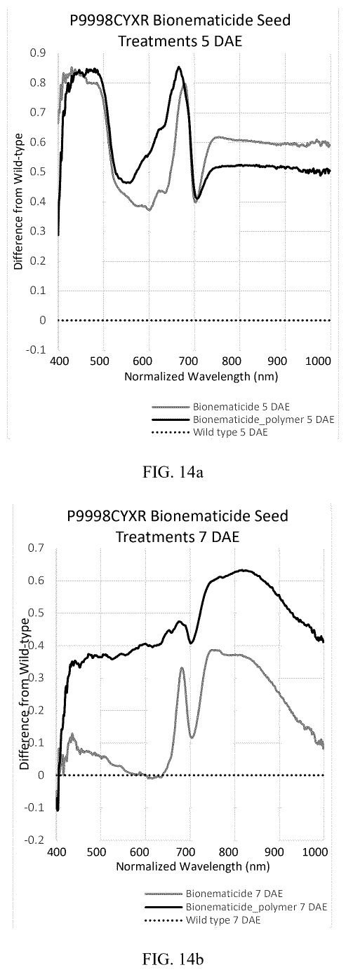

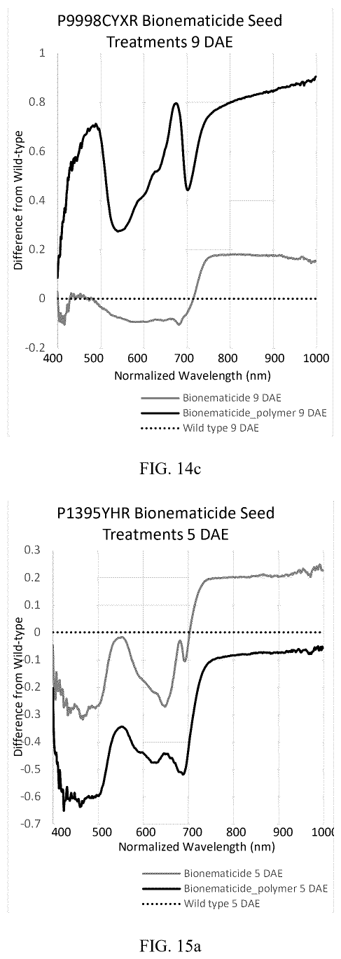

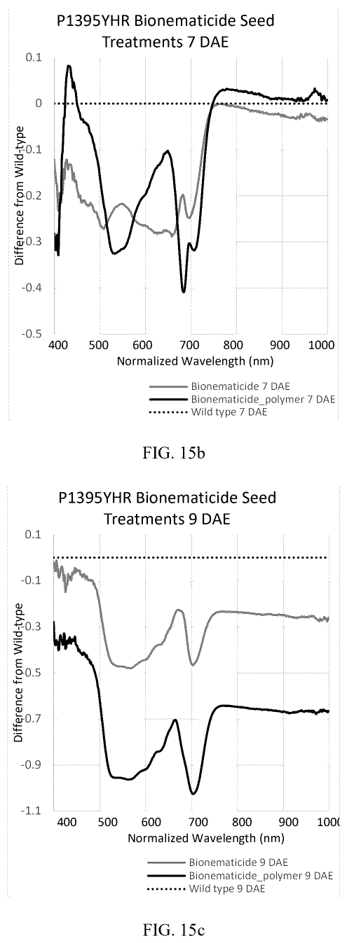

Examining the spectral reflectance patterns for corn hybrid P9998CYXR, the biological nematicide+polymer treatment had higher reflectance values when compared to the biological nematicide treatment only treatment at 7 and 9 DAE (FIG. 14). The P1395YHR hybrid had different results compared to the P9998CYXR hybrid. At 5 and 9 DAE, the biological nematicide treatment had higher values across all hyperspectral frequencies compared to the biological nematicide+polymer treatment (FIG. 15). For both hybrids, the biological nematicide treatment was closer to the wild-type control when compared to the biological nematicide+polymer treatment (FIGS. 14, 15).

The data indicate the potential to differentiate corn biological nematicide seed treatments with and without a polymer using hyperspectral imaging on seedlings. This differentiation can be seen in morphological data (pixel area, green index, yellow index, brown index) and hyperspectral reflectance signature pattern at different timepoints.

TABLE 7
Biological nematicide seed treatment components
used in corn hyperspectral experiments

| | | | | | Rate |
|---|---|---|---|---|---|
| | | | | | (mg |
| Protocol | n | Treatment | Component | Type | ai/seed) |
| Biological | 1 | Wild-type (not treated) | Not treated | | |
| nematicide, | 2 | Biological nematicide | B. amyloliquefaciens | Nematicide | 0.044 |
| Biological | | | Red Color | Colorant | 0.0552 |
| nematicide + | 3 | Biological nematicide + | B. amyloliquefaciens | Nematicide | 0.044 |
| polymer | | polymer | Polymer | Polymer | 0.167 |
| | | | Red Color | Colorant | 0.0552 |

TABLE 8
Mahalanobis distance (log transformation of standard deviations)
comparison of corn hybrids P9998CYXR and P135YHRCE treated with
biological nematicide or biological nematicide + polymer
seed treatments compared to wild-type (WT, not treated seed).

| | | Seed Treatment B | | |
|---|---|---|---|---|
| | | | | Biological |
| | | Wild-type | Biological | nematicide + |
| Genotype | Seed Treatment A | (not treated) | nematicide | polymer |
| P9998CYXR | Wild-type (not treated) | 0 | | |
| P9998CYXR | Biological nematicide | 4.7304 | 0 | |
| P9998CYXR | Biological nematicide + polymer | 4.1442 | 0.9145 | 0 |
| P1395YHR | Wild-type (not treated) | 0 | | |
| P1395YHR | Biological nematicide | 2.5552 | 0 | |
| P1395YHR | Biological nematicide + polymer | 2.5601 | 0.9669 | 0 |

### Example 4. Combination of Fungicides+Insecticides+Biologicals+Polymer Seed Treatments on Corn

### Materials and Methods:

Commercial hybrids P1138YHR and P1395YHR were selected based upon unique genetic background and adaptation to different regions of the United States. The seed treatments evaluated were combinations of different biological, fungicide, insecticide, nematicide and/or polymer seed treatment components (Table 9). Corn hybrid seed were treated as water-based slurries using a Hege 11 laboratory seed treater (Wintersteiger AG, Ried im Innkreis, Austria). Slurries were created by mixing the treatment components in a sufficient volume of tap water to ensure uniform seed coverage. Prepared slurries were pipetted onto the spinning disk of a Hege 11 seed treater in application volumes of 90-130 mL slurry per 80,000 seed for corn, and per 140,000 seeds for soybean. Seeds were circulated in treater bowl for 15-25 seconds to uniformly coat the seed. Treated seed were air dried prior to planting.

Sets of corn experiments were planted on 2 Feb. 2019 in the Corteva Agriscience (Johnston, IA) greenhouse facilities, with 10 replicates per seed treatment. Seed was planted at a standard planting depth of 20 to 25 mm in a standard germination mix comprising of 76.49% peat, 16.10% peat, 7.38% vermiculite, 5 lbs/yd³ lime, 3 lbs/yd³ starter fertilizer, and 0.5 lbs/yd³ wetting agent. The individual seeds were placed in flats of 32 plants each in an 8×4 configuration resulting in a 212 plants/m² density.

The plants were grown from planting in a glass greenhouse with growing conditions of a day temperature at 22.22 °C, a night temperature at 19.44 °C and a day length of 16 hours. Plants were imaged at first visual sight of the plumule using a Hyperspectral Visible and Near InfraRed camera and a short-wave infrared camera. The cameras capture reflectance between 400 nm and 2500 nm. Plants grown under normal conditions were imaged daily from 5 days after emergence (DAE) until approximately 12 DAE (V0 to V4 growth stage). Data were processed using proprietary software into average reflectance spectra of individual plants and physiologically relevant features. These data were standardized and processed using R. Custom algorithms were developed to adjust data and remove variability. Data were processed using a modified version of Mahalanobis distance to determine differences between treatments. Resulting distance matrices were log-transformed, with values over 3 considered significantly different.

### Example 4. Results and Discussion

The Mahalanobis distance is a measure of the distance between a datapoint and the distribution of data; quantifying the number of standard deviations the datapoint is away from the mean of the distribution. The larger the value, the further away the seed treatment is from the wild-type not treated control. The FST+IST+Polymer, FST+IST+Biological+Polymer and FST+IST+Biological nematicide seed treatments were all significantly different compared to the wild-type in both hybrids, with values ranging from 3.9 to 6.5 (Table 10). The FST+IST+Polymer treatment was significantly different compared to the FST+IST+Biological nematicide treatment in both hybrids, with values ranging from 3.6 to 3.8 (Table 10). In addition, the FST+IST+Biological nematicide was significantly different compared to the FST+IST+Biological+Polymer treatment in both hybrids (Table 10).

Morphological characteristics derived from hyperspectral imaging of total pixel area, green index, yellow index, and brown index were evaluated for different corn seed treatment combinations (Table 11). Across the different plant ages, the average total pixel area trait was selected for further elucidation of seedling vigor and early plant growth from 7 to 10 DAE (approximately V1 to V4 growth stage).

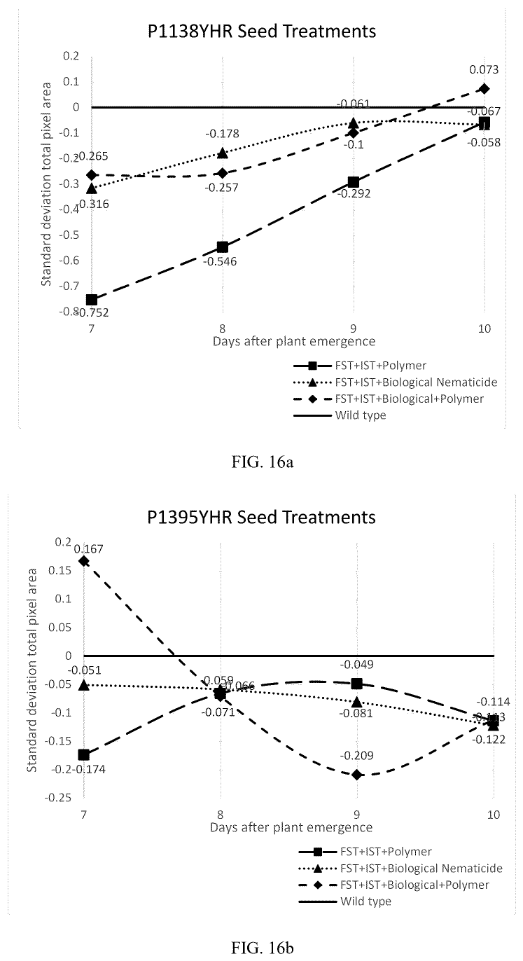

In graphing the difference in average pixel area over time for both corn hybrids P1138YHR and P1395YHR the FST+IST+Polymer treatment had the most deviation compared to control at 7 DAE and had a smaller difference at 10 DAE (FIG. 16). For hybrid P1138YHR, the FST+IST+Biological+Polymer treatment and the FST+IST+Biological nematicide treatment had higher pixel area compared to the FST+IST+Polymer treatment across all plant stages evaluated (FIG. 16). For hybrid P1395YHR the FST+IST+Biological+Polymer treatment had higher pixel area compared to control at 7 DAE, then was less than control from 8 to 10 DAE.

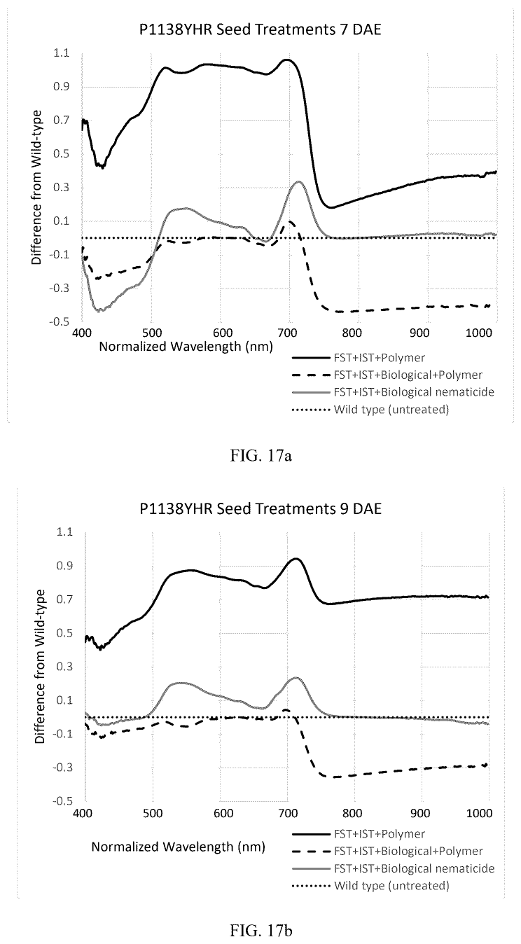

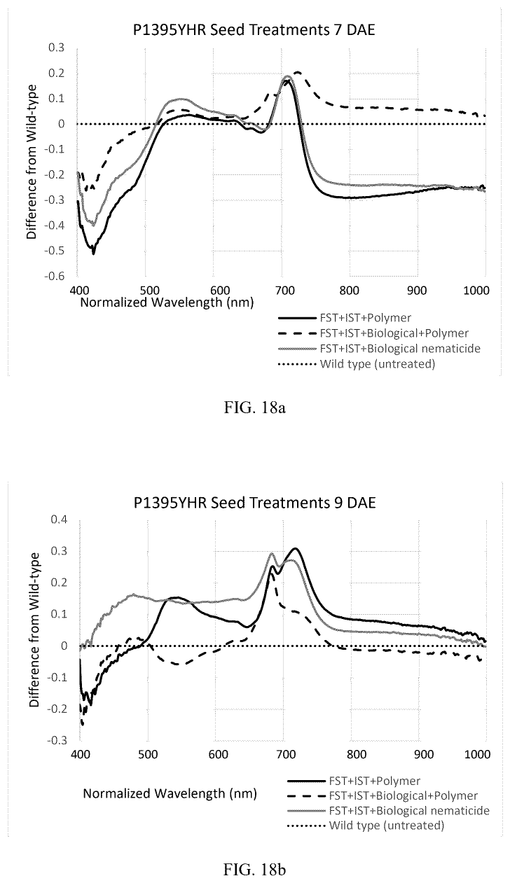

Plotting the spectral reflectance curves provides a unique fingerprint for each corn seed treatment mixture of different active ingredients. For hybrid P1138YHR, the hyperspectral wavelength patterns are similar across the three seed treatments of FST+IST+Polymer, FST+IST+Biological+Polymer, and FST+IST+Biological nematicide, relative to not treated wild-type control (FIG. 17). These similar patterns hold across 7 and 9 DAE. For hybrid P1395YHR at 7 DAE, the FST+IST+Biological+Polymer treatment had a unique pattern of reflectance, appearing to have broader peaks in the 450 nm and 725 nm regions (FIG. 18). At 9 DAE, all three treatments have regions of the spectra with unique behavior, notably the 400-500 nm region for FST+IST+Polymer and FST+IST+Biological nematicide (FIG. 18), the curved regions between 500-600 nm for all three treatments, and the different peak patterns from about 650-750 nm for FST+IST+Biological+Polymer (FIG. 18). These observations, taken together, indicate the ability for hyperspectral imaging systems to detect unique growth behaviors of reflectance spectra that can differentiate mixtures of corn seed treatment fungicides, insecticides, biologicals from one another over time.

TABLE 9
Corn seed treatments with different fungicide, insecticide, biological nematicide,
and/or polymer seed treatment mixtures used in hyperspectral experiments

| n | Treatment | Component | Type | Rate (mg ai/seed) |
|---|---|---|---|---|
| 1 | Wild-type (not treated) | Not treated | | |
| 2 | FST + IST + Polymer | Fludioxonil | Fungicide mix | 0.064 |
| | | Mefenoxam | | |
| | | Thiabendazole | | |
| | | Azoxystrobin | | |
| | | Ethaboxam | | |
| | | Clothianidin | Insecticide | 0.25 |
| | | Chlorantraniliprole | Insecticide | 0.25 |
| | | Polymer | Polymer | 0.65 |
| | | Red Color | Colorant | 0.0552 |
| 3 | FST + IST + Biological + | Fludioxonil | Fungicide mix | 0.064 |
| | Polymer | Mefenoxam | | |
| | | Thiabendazole | | |
| | | Azoxystobin | | |
| | | Ethaboxam | | |
| | | Clothianidin | Insecticide | 0.25 |
| | | Chlorantraniliprole | Insecticide | 0.25 |
| | | Biological + Polymer | Biological + Polymer | 0.419 |
| | | Red Color | Colorant | 0.0552 |
| 4 | FST + IST + Biological | Fludioxonil | Fungicide mix | 0.064 |
| | nematicide | Mefenoxam | | |
| | | Thiabendazole | | |
| | | Azoxystobin | | |
| | | Ethaboxam | | |
| | | Clothianidin | Insecticide | 0.25 |
| | | Chlorantraniliprole | Insecticide | 0.25 |
| | | Biological nematicide | Biological nematicide | 0.044 |
| | | Red Color | Colorant | 0.0552 |

TABLE 10
Mahalanobis distance (log transformation) of corn hybrids P1138YHR and P1395YHR
treated with combinations of fungicide (FST) + insecticide (IST) +
biological and/or polymer seed treatment mixtures and wild-type (WT, not treated seed)

| | | Wild-type | FST + IST + | Biological + | Biological |
|---|---|---|---|---|---|
| Hybrid | Seed Treatment A | (not treated) | Polymer | Polymer | nematicide |
| P1138YHR | Wild-type (not treated) | 0 | | | |
| P1138YHR | FST + IST + Polymer | 3.9077 | 0 | | |
| P1138YHR | FST + IST + Biological + Polymer | 4.8813 | 2.8326 | 0 | |
| P1138YHR | FST + IST + Biological nematicide | 6.0959 | 3.7782 | 3.1956 | 0 |
| P1395YHR | Wild-type (not treated) | 0 | | | |
| P1395YHR | FST + IST + Polymer | 6.5244 | 0 | | |
| P1395YHR | FST + IST + Biological + Polymer | 5.5774 | 3.5123 | 0 | |
| P1395YHR | FST + IST + Biological nematicide | 4.1441 | 3.6027 | 3.5154 | 0 |

FST = fungicide seed treatment, IST = insecticide seed treatment

TABLE 11
Hyperspectral differences¹ of corn hybrids P1138YHR or P1395YHR treated with combinations of fungicide (FST) +
insecticide (IST) + biological and/or polymer seed treatment mixtures compared to WT (wild-type not treated control)

| | | P1138YHR | | | | P1395YHR | | | |
|---|---|---|---|---|---|---|---|---|---|
| | | Avg | Avg | Avg | Avg | Avg | Avg | Avg | Avg |
| Plant | | Pixel | Green | Yellow | Brown | Pixel | Green | Yellow | Brown |
| age | Seed Treatment Comparison | Area | Index | Index | Index | Area | Index | Index | Index |
| 7 | (FST + IST + Polymer) - WT | −0.752 | −0.848 | 0.753 | −0.58 | −0.174 | −0.624 | 1.311 | −0.793 |
| 7 | (FST + IST + Biological + Polymer) - WT | −0.265 | −0.358 | 0.834 | −0.173 | 0.167 | −0.366 | 0.173 | −0.596 |
| 7 | (FST + IST + Biological nematicide) - WT | −0.316 | −0.295 | 1.27 | −0.306 | −0.051 | −1.223 | −0.135 | −1.049 |
| 8 | (FST + IST + Polymer) - WT | −0.546 | −1.176 | 0.984 | −1.12 | −0.066 | −0.702 | 0.031 | −0.859 |
| 8 | (FST + IST + Biological + Polymer) - WT | −0.257 | −0.571 | −0.115 | −0.766 | −0.071 | −1.047 | 1.516 | −1.177 |
| 8 | (FST + IST + Biological nematicide) - WT | −0.178 | −0.691 | 1.256 | −1.006 | −0.059 | −0.611 | 0.124 | −0.468 |
| 9 | (FST + IST + Polymer) - WT | −0.292 | −0.344 | −1.071 | 0.033 | −0.049 | −0.64 | −0.048 | −0.895 |
| 9 | (FST + IST + Biological + Polymer) - WT | −0.100 | −0.214 | 0.269 | −0.468 | −0.209 | −1.285 | 1.492 | −1.556 |
| 9 | (FST + IST + Biological nematicide) - WT | −0.061 | −1.054 | 1.484 | −1.322 | −0.081 | −0.471 | −0.098 | −0.44 |
| 10 | (FST + IST + Polymer) - WT | −0.058 | −0.801 | 0.179 | −0.959 | −0.114 | −0.189 | −0.671 | −0.411 |
| 10 | (FST + IST + Biological + Polymer) - WT | 0.073 | 0.146 | 0.127 | −0.05 | −0.113 | −1.267 | 1.296 | −0.922 |
| 10 | (FST + IST + Biological nematicide) - WT | −0.067 | −1.409 | 1.66 | −1.651 | −0.122 | −0.252 | 0.064 | 0.431 |

### Example 5. Soybean Fungicide Seed Treatments

### Materials and Methods:

Soybean varieties P31A22X and P48A60X were selected based upon unique genetic background and adaptation to northern or southern regions of the United States. Soybean seed were treated with different fungicide seed treatments (FST) (Table 12) as water-based slurries using a Hege 11 laboratory seed treater (Wintersteiger AG, Ried im Innkreis, Austria). Slurries were created by mixing the treatment components in a sufficient volume of tap water to ensure uniform seed coverage. Prepared slurries were pipetted onto the spinning disk of a Hege 11 seed treater in application volumes of 90-130 mL slurry per 140,000 seeds for soybean. Seeds were circulated in treater bowl for 15-25 seconds to uniformly coat the seed. Treated seed were air dried prior to planting.

Soybean FST experiments were planted in the Corteva Agriscience (Johnston, IA) greenhouse facilities, with two different planting dates of 10 replicates per seed treatment in each planting date. Planting date 1 was 26 Aug. 2020 and planting date 2 was 22 Feb. 2020. Seed was planted at a standard planting depth of 20 to 25 mm in a standard germination mix comprising of 76.49% peat, 16.10% peat, 7.38% vermiculite, 5 lbs/yd³ lime, 3 lbs/yd³ starter fertilizer, and 0.5 lbs/yd³ wetting agent. The individual seeds were placed in flats of 32 plants each in an 8×4 configuration resulting in a 212 plants/m² density.

The plants were grown in a glass greenhouse with of a day length of 16 hours and growth conditions of daytime temperature at 22.22 °C, and nighttime temperature at 19.44 °C. Plants were imaged at first visual sight of the plumule using a Hyperspectral Visible and Near InfraRed camera and a short-wave infrared camera. The cameras capture reflectance between 400 nm and 2500 nm. Plants grown under normal conditions were imaged daily from 5 days after emergence (DAE) until approximately 12 DAE (V0 to V4 growth stage). Data were processed using proprietary software into average reflectance spectra of individual plants and physiologically relevant features. These data were standardized and processed using R. Custom algorithms were developed to adjust data and remove variability. Data were processed using a modified version of Mahalanobis distance to determine differences between treatments. Resulting distance matrices were log-transformed, with values over 3 considered significantly different.

### Results and Discussion:

The Mahalanobis distance is a measure of the distance between a datapoint and the distribution of data; quantifying the number of standard deviations the datapoint is away from the mean of the distribution. The larger the value, the further away the fungicide seed treatment is from the wild-type not treated control. The log transformation of Mahalanobis distance revealed fluopyram and fluopyram+polymer were two FST that were the most different compared to the wild-type control with values greater than 3.2 for both varieties (Table 13). The oxathiapiprolin and picoxystrobin (with and without polymer) were not different compared to wild-type control, with values less than 2.8 for both varieties (Table 13). The fluopyram treatment was different compared to all other FST (with and without polymer), with values greater than 3 for all paired comparisons (Table 13). The fluopyram+polymer treatment was different (values greater than 3) compared to all the treatments except picoxystrobin+polymer on the P48A60X variety (Table 13). For both varieties, a single FST compared to the same FST with a polymer was not significantly different, with values less than 1 (Table 13).

For visible hyperspectral characteristics, the average pixel area trait had the most magnitude of differences among all the plant ages tested for both P31A22X and P48A60X (Table 14). The green, yellow, and brown indices were different for treatments at ages 5 and 10-11, but not much variability was detected from ages 6 to 9 DAE (Table 14). Fluopyram and fluopyram+polymer treatments were consistently lower compared to the other FST (with and without a polymer) across all plant ages for both soybean varieties (Table 14).

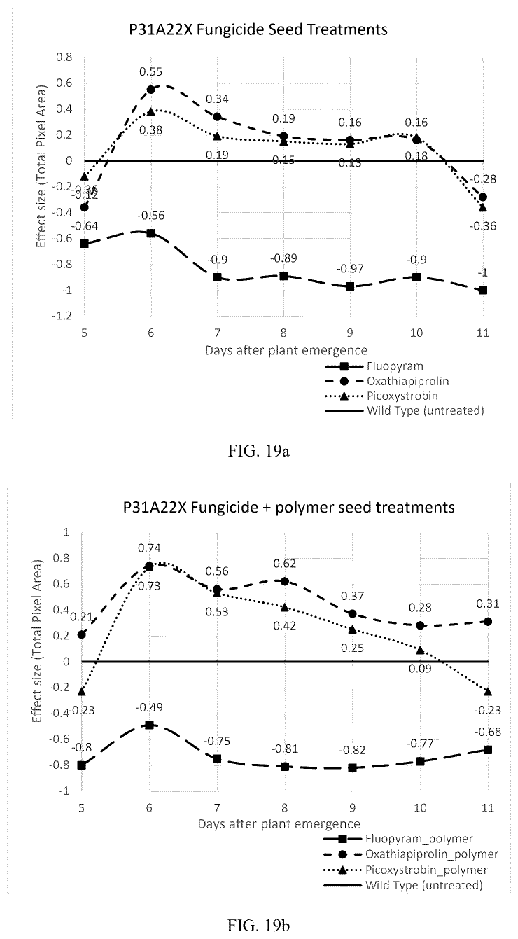

For the P31A22X variety, when average pixel area data were graphed, the fluopyram and fluopyram+polymer treatments were both lower than wild-type control for all growth stages (FIG. 19). The oxathiapiprolin, picoxystrobin, and picoxystrobin+polymer treatments started less than control at 5 DAE, were higher than control from 6 to 10 DAE, and less than control 11 DAE (FIG. 19). For each FST, adding a polymer increased the pixel area values compared to the corresponding individual fungicide for each plant growth stage (FIG. 19).

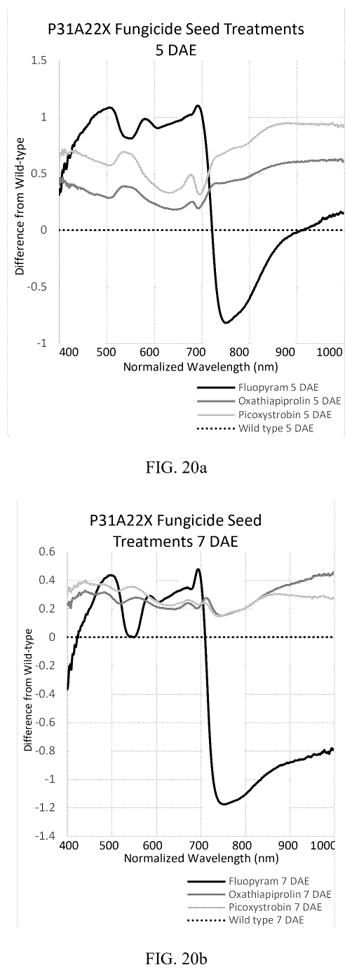

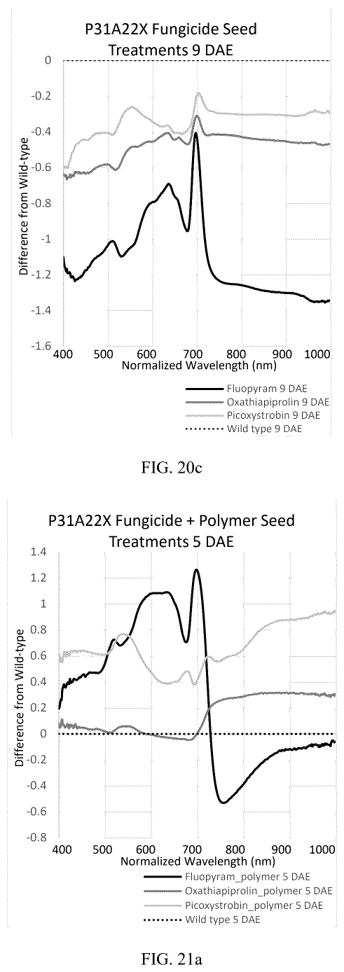

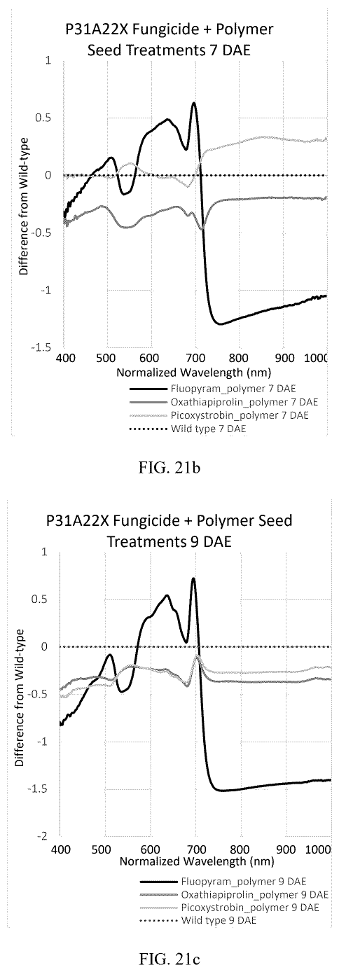

The hyperspectral differences between a FST and control were graphed for soybean variety P31A22X (FIG. 20). Oxathiapiprolin and picoxystrobin treatments generally followed the same spectral reflectance patterns across ages, while fluopyram had a distinct and very unique pattern (FIG. 20). The strong cross in relative reflectance at 700 nm to 750 nm is diminished in the latest stage (9 DAE), though still present despite all experimental treatment conditions having values lower than WT (FIG. 20). These observed trends remained when a polymer was added to these fungicides, with fluopyram+polymer remaining unique in terms of spectral reflectance pattern compared to oxathiapiprolin+polymer or picoxystrobin+polymer (FIG. 21).

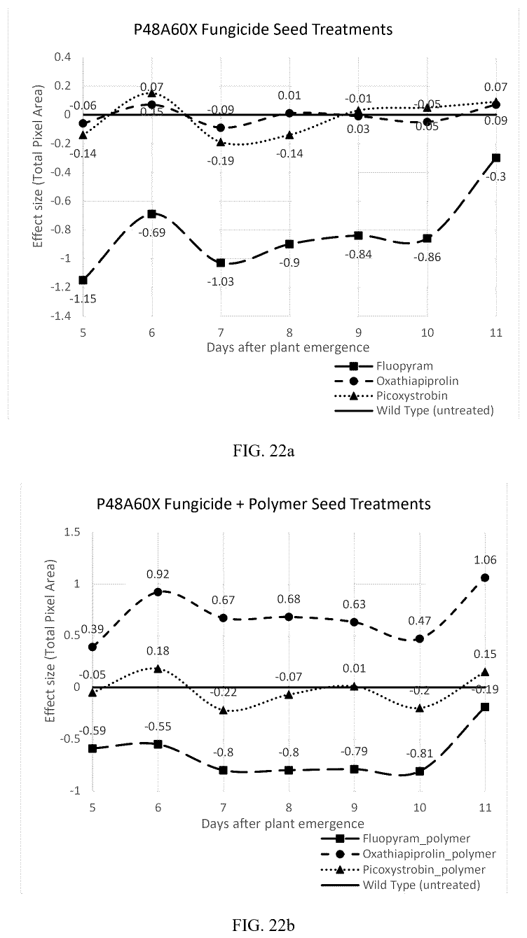

The average pixel area for fluopyram and fluopyram+polymer treatments were less than the wild-type not treated control for the P48A60X variety (FIG. 22). The oxathiapiprolin+polymer treatment had more pixel area compared to the wild-type control for all the growth stages tested (FIG. 22). The addition of the polymer generally increased the pixel area at every timepoint for each FST when compared to the fungicide alone (FIG. 22).

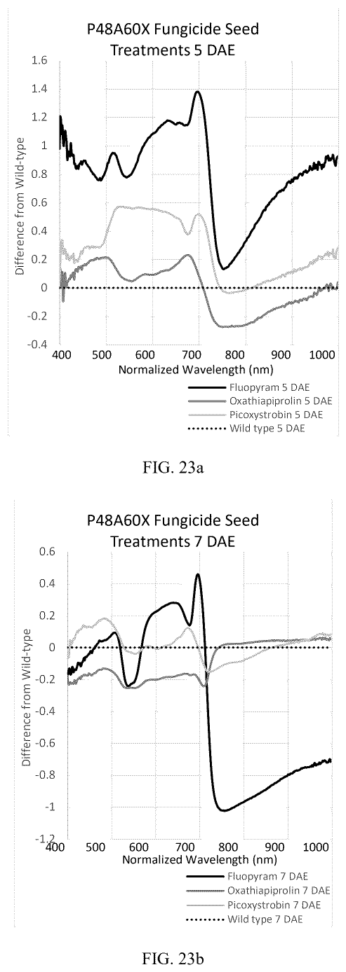

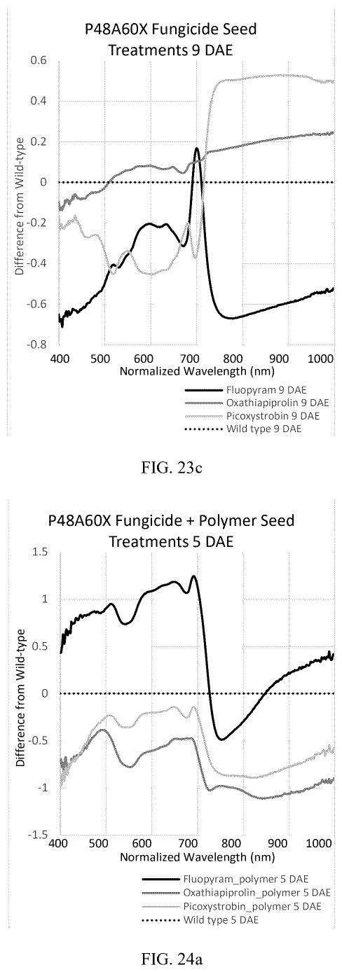

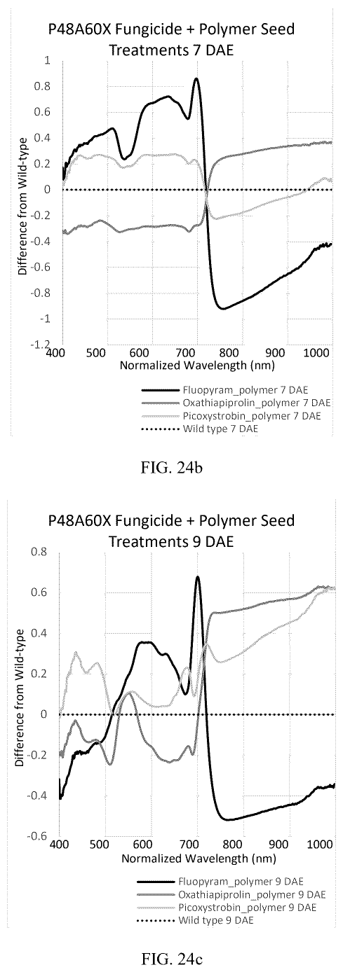

For soybean variety P48A60X, the fluopyram treatment had a spectral reflectance pattern similar to the pattern observed for soybean variety P31A22X (FIGS. 20, 23; 21, 24). This pattern is distinct from the other two fungicide treatments and is hallmarked by the strong relative decrease in reflectance in the 700 nm to 750 nm range. Across both varieties, the picoxystrobin seed treatment in general had higher positive difference values compared to the oxathiapiprolin seed treatment (FIGS. 20, 23). When a polymer was added, picoxystrobin+polymer consistently had higher pixel values compared to oxathiapiprolin+polymer at all wavelengths for P31A22X, and generally for wavelengths greater than 700 nm for P48A60X across all growth stages (FIGS. 21, 24).

The data indicate the potential to differentiate soybean fungicide seed treatments with and without a polymer using hyperspectral imaging on seedlings. This differentiation can be seen in morphological data (pixel area, green index, yellow index, brown index) and unique hyperspectral reflectance signature patterns for each treatment at different timepoints.

TABLE 12
Seed treatment components used in soybean hyperspectral experiments

| Protocol | n | Treatment | Component | Type | Rate (mg ai/seed) |
|---|---|---|---|---|---|
| Fungicide, | 1 | Wild-type (not treated) | Not treated | | |
| Fungicide + | 2 | Oxathiapiprolin | Oxathiapiprolin | Fungicide | 0.012 |
| polymer | | | Green Color | Colorant | 0.02 |
| | 3 | Picoxystrobin | Picoxystrobin | Fungicide | 0.011 |
| | | | Green Color | Colorant | 0.02 |
| | 4 | Fluopyram 0.095 | Fluopyram | Fungicide | 0.095 |
| | | | Green Color | Colorant | 0.02 |
| | 5 | Oxathiapiprolin + polymer | Oxathiapiprolin | Fungicide | 0.010 |
| | | | Polymer | Polymer | 0.04 |
| | | | Green Color | Colorant | 0.02 |
| | 6 | Picoxystrobin + polymer | Picoxystrobin | Fungicide | 0.011 |
| | | | Polymer | Polymer | 0.04 |
| | | | Green Color | Colorant | 0.02 |
| | 7 | Fluopyram 0.095 + polymer | Fluopyram | Fungicide | 0.095 |
| | | | Polymer | Polymer | 0.04 |
| | | | Green Color | Colorant | 0.02 |

TABLE 13
Mahalanobis distance (log transformation of standard deviations) of soybean varieties P31A22X or P48A60X
treated with fungicide or fungicide + polymer seed treatments compared to wild-type (WT, not treated seed)

| | | Seed Treatment B | | | | | | |
|---|---|---|---|---|---|---|---|---|
| | | Wild-type | | | | Oxathia- | | Picoxy- |
| | | (not | | Fluopyram + | Oxathia- | piprolin + | Picoxy- | strobin + |
| Genotype | Seed Treatment A | treated) | Fluopyram | polymer | piprolin | polymer | strobin | polymer |
| P31A22X | Wild-type (not treated) | 0 | | | | | | |
| P31A22X | Fluopyram | 5.3003 | 0 | | | | | |
| P31A22X | Fluopyram + polymer | 5.2217 | −0.9166 | 0 | | | | |
| P31A22X | Oxathiapiprolin | 2.7617 | 5.1472 | 5.1034 | 0 | | | |
| P31A22X | Oxathiapiprolin + | 2.3342 | 4.3260 | 4.4123 | 0.5955 | 0 | | |
| | polymer | | | | | | | |
| P31A22X | Picoxystrobin | 1.5506 | 4.6447 | 4.7345 | 1.5661 | 2.1036 | 0 | |
| P31A22X | Picoxystrobin + | 1.7975 | 4.8028 | 4.8364 | 1.4342 | 1.6736 | −0.6039 | 0 |
| | polymer | | | | | | | |
| P48A60X | Wild-type (not treated) | 0 | | | | | | |
| P48A60X | Fluopyram | 3.6903 | 0 | | | | | |
| P48A60X | Fluopyram + polymer | 3.2552 | 0.3496 | 0 | | | | |
| P48A60X | Oxathiapiprolin | 0.9124 | 3.3808 | 3.1927 | 0 | | | |
| P48A60X | Oxathiapiprolin + | 2.3371 | 3.4450 | 3.0389 | 1.3184 | 0 | | |
| | polymer | | | | | | | |
| P48A60X | Picoxystrobin | 2.7752 | 3.3135 | 3.2452 | 2.7152 | 2.7318 | 0 | |
| P48A60X | Picoxystrobin + | 2.6840 | 3.0217 | 2.9195 | 2.5357 | 2.5226 | 0.3672 | 0 |
| | polymer | | | | | | | |

TABLE 14
Hyperspectral differences¹ of soybean varieties P31A22X or P48A60X treated with fungicide
or fungicide + polymer seed treatments compared to WT (wild-type not treated control)

| | | P31A22X | | | | P48A60X | | | |
|---|---|---|---|---|---|---|---|---|---|
| | | Avg | Avg | Avg | Avg | Avg | Avg | Avg | Avg |
| Plant | | Pixel | Green | Yellow | Brown | Pixel | Green | Yellow | Brown |
| age | Label | Area | Index | Index | Index | Area | Index | Index | Index |
| 5 | Fluopyram - WT | −4.6 | −46.7 | 25.8 | 158 | −8.1 | −197 | 0.8 | 1029.2 |
| 5 | Fluopyram polymer - WT | −5.8 | −48.3 | 60.7 | 161 | −4.2 | −239.3 | 1.5 | 1274.9 |
| 5 | Oxathiapiprolin - WT | −3.2 | −15.6 | 25.6 | −2.1 | −1.9 | −128.8 | −83.7 | 258 |
| 5 | Oxathiapiprolin polymer - WT | 0.7 | 16.1 | 77.4 | −39.7 | 2.8 | −139.6 | −91.6 | 245 |
| 5 | Picoxystrobin - WT | −1.2 | −9.9 | 336.9 | 24.4 | −3.2 | −319.1 | 6.2 | 440.4 |
| 5 | Picoxystrobin polymer - WT | −2.4 | −10.8 | 326.8 | 26.7 | −2.7 | −318.5 | 5.9 | 438.8 |
| 6 | Fluopyram - WT | −5.9 | −16.6 | 10.4 | 21 | −5.1 | −13.3 | 4.7 | 16.2 |
| 6 | Fluopyram polymer - WT | −5.1 | −16.1 | 10.1 | 20.3 | −4.1 | −13.3 | 4.5 | 15.7 |
| 6 | Oxathiapiprolin - WT | 5.5 | −4.1 | −2.6 | 3.7 | 0.1 | −0.8 | 0.8 | −1.3 |
| 6 | Oxathiapiprolin polymer - WT | 6.7 | −3.9 | −1.5 | 3 | 5.6 | −1.5 | 1.5 | −1.2 |
| 6 | Picoxystrobin - WT | 3.9 | −8.8 | −3.8 | 9.5 | 1 | −7.2 | 1 | 4.6 |
| 6 | Picoxystrobin polymer - WT | 7.3 | −7.7 | −4.5 | 8.4 | 0.7 | −3.8 | 0 | 0.3 |
| 7 | Fluopyram - WT | −9.3 | −30 | −2 | 24.6 | −9.3 | −8.1 | 37.7 | 8.7 |
| 7 | Fluopyram polymer - WT | −7.7 | −29.6 | 2.2 | 24.7 | −7.2 | −7.9 | 42.7 | 8.3 |
| 7 | Oxathiapiprolin - WT | 3.1 | −3.9 | −3.3 | 4.6 | −1.3 | −2 | −0.6 | 2.1 |
| 7 | Oxathiapiprolin polymer - WT | 4.9 | −5.6 | −3.2 | 5.4 | 5.7 | −1.8 | 1.2 | 1.8 |
| 7 | Picoxystrobin - WT | 1.9 | −8.4 | −2.7 | 6.4 | −1.7 | 2.8 | 8 | −3 |
| 7 | Picoxystrobin polymer - WT | 4.9 | −9.1 | −3.2 | 6.5 | −2.7 | 2.3 | 6.7 | −2.6 |
| 8 | Fluopyram - WT | −9.3 | −42 | 2.7 | 30.7 | −7.8 | −6.1 | 33.3 | 6.4 |
| 8 | Fluopyram polymer - WT | −8.4 | −41.3 | 3 | 31 | −7 | −5 | 33.5 | 5.3 |
| 8 | Oxathiapiprolin - WT | 1.8 | −7.5 | −3.2 | 9.1 | −0.2 | −4.4 | −7.6 | 4.5 |
| 8 | Oxathiapiprolin polymer - WT | 5.7 | −6.8 | −2.6 | 7.5 | 5.9 | −3.4 | −5.4 | 3.5 |
| 8 | Picoxystrobin - WT | 1.5 | 4.6 | 0.1 | 2.1 | −1.2 | 4.2 | 3.1 | −4.3 |
| 8 | Picoxystrobin polymer - WT | 4.3 | 3.9 | −0.1 | 2.3 | −1.3 | 3.2 | 1.2 | −3.2 |
| 9 | Fluopyram - WT | −9.6 | −9.3 | 1.4 | 13.5 | −7.1 | −0.2 | 10.6 | 0.3 |
| 9 | Fluopyram polymer - WT | −8.1 | −9.6 | 1.9 | 13.3 | −6.7 | 0.8 | 11.4 | −0.1 |
| 9 | Oxathiapiprolin - WT | 1.5 | −3.2 | −1.4 | 5.7 | −0.1 | −8.7 | −6.4 | 8.1 |
| 9 | Oxathiapiprolin polymer - WT | 3.3 | −3.3 | −1.4 | 4.9 | 5.3 | −4.5 | −5.1 | 5 |
| 9 | Picoxystrobin - WT | 1.3 | 7.8 | 2 | 2.4 | 0.1 | 7.4 | −6 | −4.1 |
| 9 | Picoxystrobin polymer - WT | 2.5 | 8.4 | 1.5 | 2.5 | −0.5 | 5.7 | −8 | −2.8 |
| 10 | Fluopyram - WT | −8.1 | −12.3 | 6.9 | 4.1 | −7.7 | 4.8 | 25.5 | −11.8 |
| 10 | Fluopyram polymer - WT | −6.9 | −11.1 | 6.8 | 3.9 | −7.3 | 5.5 | 24.1 | −11.5 |
| 10 | Oxathiapiprolin - WT | 1.2 | −6.4 | −2 | 5.9 | −0.4 | −0.9 | 3.7 | −7.4 |
| 10 | Oxathiapiprolin polymer - WT | 2.5 | −8.1 | −0.3 | 6.1 | 4.3 | −0.2 | 3.4 | −6.5 |
| 10 | Picoxystrobin - WT | 1.4 | 19.7 | 6.4 | −1.7 | 0.2 | 10.8 | 3.3 | −15.8 |
| 10 | Picoxystrobin polymer - WT | 0.8 | 17.5 | 6.8 | −1 | −2.3 | 9.9 | 0.3 | −13.2 |
| 11 | Fluopyram - WT | −7.1 | −37.8 | 167.4 | −26.5 | −2.7 | −6.6 | 13.9 | −42.6 |
| 11 | Fluopyram polymer - WT | −4.8 | −33.2 | 176.3 | −30.9 | −1.7 | −6.6 | 21.7 | −43.2 |
| 11 | Oxathiapiprolin - WT | −2 | −32.8 | 25.5 | 0 | 0.6 | −14.1 | −21.8 | −24.9 |
| 11 | Oxathiapiprolin polymer - WT | 2.2 | −24.5 | 23.2 | 1.6 | 9.3 | −7.6 | −20.9 | −16.9 |
| 11 | Picoxystrobin - WT | −2.5 | 48.6 | 105.2 | −13.1 | 0.5 | 20.8 | −28.6 | −44.1 |
| 11 | Picoxystrobin polymer - WT | −1.7 | 42.6 | 106.5 | −11 | 1 | 16.8 | −72.6 | −20.3 |

¹Positive values indicate better performance compared to WT (wild-type not treated control), negative values indicate worse performance compared to WT.

### Example 6. Soybean Insecticide Seed Treatments

### Materials and Methods:

Soybean varieties P31A22X and P48A60X were selected based upon unique genetic background and adaptation to northern or southern regions of the United States. Soybean seed were treated with different insecticide seed treatments (IST) (Table 15) as water-based slurries using a Hege 11 laboratory seed treater (Wintersteiger AG, Ried im Innkreis, Austria). Slurries were created by mixing the treatment components in a sufficient volume of tap water to ensure uniform seed coverage. Prepared slurries were pipetted onto the spinning disk of a Hege 11 seed treater in application volumes of 90-130 mL slurry per 140,000 seeds for soybean. Seeds were circulated in treater bowl for 15-25 seconds to uniformly coat the seed. Treated seed were air dried prior to planting.

Soybean IST hyperspectral experiments were planted in the Corteva Agriscience (Johnston, IA) greenhouse facilities, with two different planting dates of 10 replicates per seed treatment in each planting date. Planting date 1 was 16 Sep. 2020 and planting date 2 was 1 Feb. 2020. Seed was planted at a standard planting depth of 20 to 25 mm in a standard germination mix comprising of 76.49% peat, 16.10% peat, 7.38% vermiculite, 5 lbs/yd³ lime, 3 lbs/yd³ starter fertilizer, and 0.5 lbs/yd³ wetting agent. The individual seeds were placed in flats of 32 plants each in an 8×4 configuration resulting in a 212 plants/m² density.

The plants were grown in a glass greenhouse with of a day length of 16 hours and growth conditions of daytime temperature at 22.22 °C, and nighttime temperature at 19.44 °C. Plants were imaged at first visual sight of the plumule using a Hyperspectral Visible and Near InfraRed camera and a short-wave infrared camera. The cameras capture reflectance between 400 nm and 2500 nm. Plants grown under normal conditions were imaged daily from 5 days after emergence (DAE) until approximately 10 DAE (V0 to V3 growth stage). Data were processed using proprietary software into average reflectance spectra of individual plants and physiologically relevant features. These data were standardized and processed using R. Custom algorithms were developed to adjust data and remove variability. Data were processed using a modified version of Mahalanobis distance to determine differences between treatments. Resulting distance matrices were log-transformed, with values over 3 considered significantly different.

### Results and Discussion:

The Mahalanobis distance is a measure of the distance between a datapoint and the distribution of data; quantifying the number of standard deviations the datapoint is away from the mean of the distribution. The larger the value, the further away the seed treatment is from the wild-type not treated control. For cyantraniliprole and cyantraniliprole+polymer seed treatments, the log transformation of Mahalanobis distance was significantly different compared to the wild-type control, with values greater than 3.2 for both varieties (Table 16). The cyantraniliprole treatment was not different compared to cyantraniliprole+polymer for both varieties, with values less than 1.4 (Table 16).

Evaluating the visible hyperspectral differences, the average pixel area had the most magnitude of differences between treatments for both varieties tested from 5 to 10 DAE (Table 17). The cyantraniliprole+polymer treatment had higher average pixel area values compared to cyantraniliprole along for all plant growth stages across both varieties (Table 17). The green, yellow, and brown indices had differences detected at 5 and 10 DAE, and minimal differences at 6-9 DAE (Table 17).

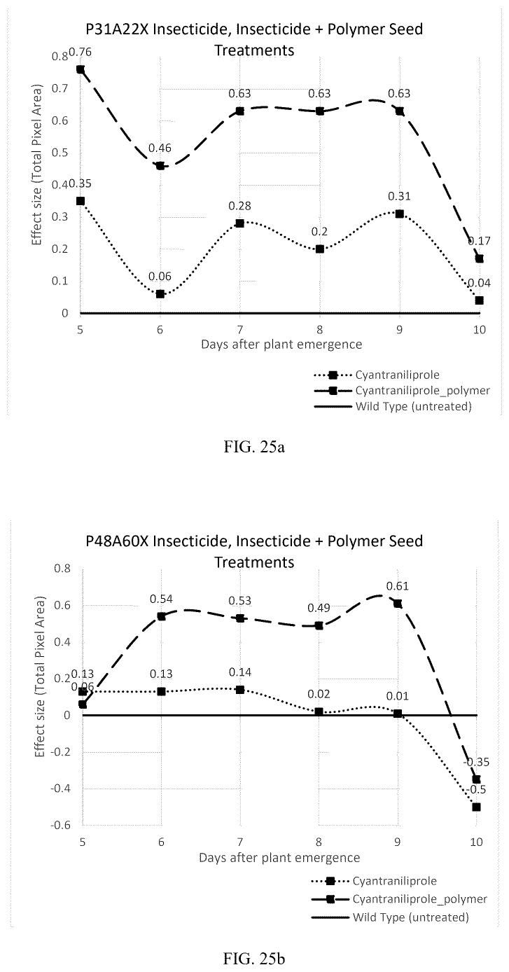

When the average pixel area data were graphed, the cyantraniliprole and cyantraniliprole+polymer treatments had better performance compared to wild-type control across all plant growth stages for soybean variety P31A22X (FIG. 25). For the P48A60X variety, cyantraniliprole and cyantraniliprole+polymer had greater pixel area compared to the wild-type control from 5 days to 9 DAE and less pixel area at 10 DAE (FIG. 25). For both the P31A22X and P48A60X varieties, the addition of the polymer to cyantraniliprole increased the pixel area values when compared to cyantraniliprole alone treatment (FIG. 25).

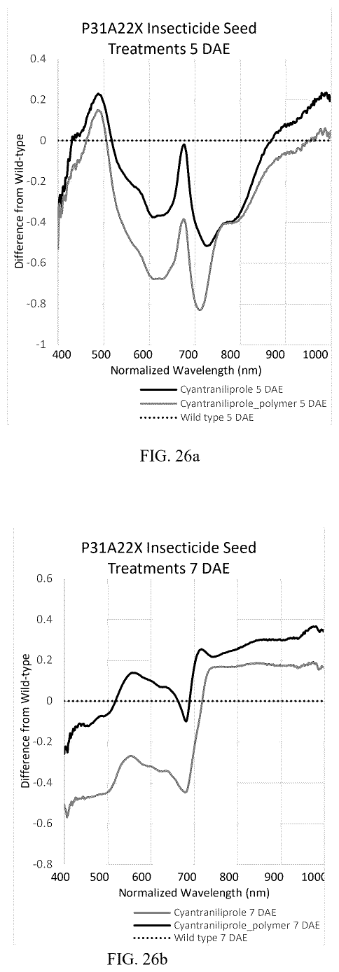

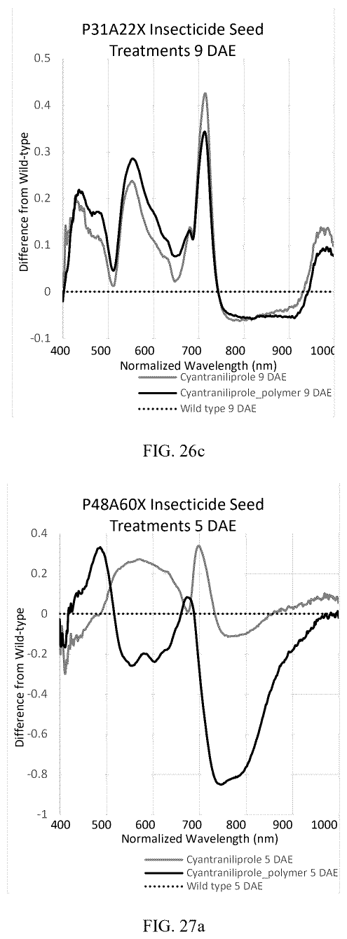

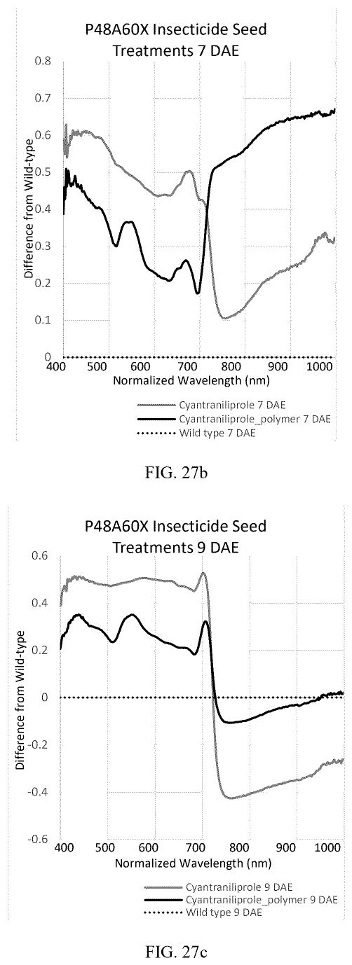

For soybean variety P31A22X, presence or absence of the polymer had little impact on the overall spectral patterns, particularly at 9 DAE (FIG. 26). However, soybean variety P48A60X had more variable performance when the cyantraniliprole and cyantraniliprole+polymer treatments are compared. At 5 DAE, the cyantraniliprole treatment generally had higher values compared to the cyantraniliprole+polymer treatment (FIG. 26). At 7 and 9 DAE, at frequencies less than 760 nm, the cyantraniliprole treatment had higher values compared to the cyantranilprole+polymer treatment (FIG. 26). The cyantraniliprole+polymer treatment had higher values compared to the cyantraniliprole treatment for frequencies greater than 760 nm at both 7 and 9 DAE (FIG. 26).

The data indicate the potential to differentiate soybean insecticide seed treatments with and without a polymer using hyperspectral imaging on early seedling growth. This differentiation can be seen in morphological data (pixel area, green index, yellow index, brown index) and hyperspectral reflectance signature pattern at different timepoints.

TABLE 15
Insecticide seed treatment components used in soybean hyperspectral evaluations

| Protocol | n | Treatment | Component | Type | Rate (mg ai/seed) |
|---|---|---|---|---|---|
| Insecticide, | 1 | Wild-type (not treated) | Not treated | | |
| Insecticide + | 2 | Cyantraniliprole | Cyantraniliprole | Insecticide | 0.075 |
| polymer | | | Green Color | Colorant | 0.02 |
| | 3 | Polymer | Polymer | Polymer | 0.04 |
| | | | Green Color | Colorant | 0.02 |
| | 4 | Cyantraniliprole + polymer | Cyantraniliprole | Insecticide | 0.075 |
| | | | Polymer | Polymer | 0.04 |
| | | | Green Color | Colorant | 0.02 |

TABLE 16
Mahalanobis distance (log transformation of standard deviations) of soybean
varieties P31A22X or P48A60X treated with insecticide or insecticide +
polymer seed treatments compared to WT (wild-type, not treated)

| | | Seed Treatment B | | |
|---|---|---|---|---|
| | | Wild-type | | Cyantraniliprole + |
| Genotype | Seed Treatment A | (not treated) | Cyantraniliprole | polymer |
| P31A22X | Wild-type (not treated) | 0 | | |
| P31A22X | Cyantraniliprole | 4.1478 | 0 | |
| P31A22X | Cyantraniliprole + polymer | 3.4843 | 0.0303 | 0 |
| P48A60X | Wild-type (not treated) | 0 | | |
| P48A60X | Cyantraniliprole | 3.1962 | 0 | |
| P48A60X | Cyantraniliprole + polymer | 3.2753 | 1.3524 | 0 |

TABLE 17
Hyperspectral differences¹ of soybean varieties P31A22X or P48A60X treated with insecticide
or insecticide + polymer seed treatments compared to WT (wild-type not treated control)

| | | P31A22X | | | | P48A60X | | | |
|---|---|---|---|---|---|---|---|---|---|
| | | Avg | Avg | Avg | Avg | Avg | Avg | Avg | Avg |
| Plant | | Pixel | Green | Yellow | Brown | Pixel | Green | Yellow | Brown |
| age | Label | Area | Index | Index | Index | Area | Index | Index | Index |
| 5 | Cyantraniliprole - WT | 1.7 | 37.8 | −745.1 | −142.7 | −0.5 | −35.9 | −96.6 | 115.2 |
| 5 | Cyantraniliprole polymer - WT | 3.7 | 34.8 | −736.3 | −134.9 | −0.2 | −41.3 | −88.5 | 132.6 |
| 6 | Cyantraniliprole - WT | 0 | 1.6 | −6.1 | −2.5 | 0.9 | 5 | 6 | −4.1 |
| 6 | Cyantraniliprole polymer - WT | 3.8 | 1.7 | −4.4 | −0.4 | 4.4 | 5.7 | 6.4 | −5.3 |
| 7 | Cyantraniliprole - WT | 2.3 | 3.7 | −7.6 | −1.5 | 0.7 | −2.5 | −1.9 | 5 |
| 7 | Cyantraniliprole polymer - WT | 6 | 1.5 | −9.5 | 0.5 | 4.3 | −5.8 | −3.3 | 8 |
| 8 | Cyantraniliprole - WT | 1.9 | 4.2 | −2.4 | −0.8 | 0.2 | −3.7 | −2.3 | 5.4 |
| 8 | Cyantraniliprole polymer - WT | 5.9 | 3.5 | 0.8 | −1.4 | 4.2 | −2.3 | −1.8 | 3 |
| 9 | Cyantraniliprole - WT | 3.2 | 3.4 | −4.3 | −1.1 | 0.1 | −5.6 | −1.6 | 7.2 |
| 9 | Cyantraniliprole polymer - WT | 6.4 | 4.8 | −1.8 | −4.7 | 5.7 | −2.3 | −2.2 | 7.2 |
| 10 | Cyantraniliprole - WT | 0.2 | 10.5 | 14.6 | −121.3 | −6.2 | −1.4 | 24.7 | 51.9 |
| 10 | Cyantraniliprole polymer - WT | 0.8 | 12.2 | 15.8 | −117.1 | −2.6 | 6.3 | 11.3 | 69.9 |

¹Positive values indicate better performance compared to WT (wild-type not treated control), negative values indicate worse performance compared to WT.

### Example 7. Combination of Fungicides+Insecticides+Biologicals+Polymer Seed Treatment Mixtures on Soybean

### Materials and Methods:

Soybean varieties P31A22X and P72A21X were selected based upon unique genetic background and adaptation to northern or southern regions of the United States. Soybean seed were treated with different combinations of fungicides, insecticides, biological nematicides, mixtures with or without a polymer (Table 18). Seed treatments were water-based slurries using a Hege 11 laboratory seed treater (Wintersteiger AG, Ried im Innkreis, Austria). Slurries were created by mixing the treatment components in a sufficient volume of tap water to ensure uniform seed coverage. Prepared slurries were pipetted onto the spinning disk of a Hege 11 seed treater in application volumes of 90-130 mL slurry per 140,000 seeds for soybean. Seeds were circulated in treater bowl for 15-25 seconds to uniformly coat the seed. Treated seed were air dried prior to planting.

Soybean hyperspectral experiments were planted in the Corteva Agriscience (Johnston, IA) greenhouse facilities, with two different planting dates of 10 replicates per seed treatment in each planting date. Planting date 1 was 2 Feb. 2019 and planting date 2 was 19 Feb. 2019. Seed was planted at a standard planting depth of 20 to 25 mm in a standard germination mix comprising of 76.49% peat, 16.10% peat, 7.38% vermiculite, 5 lbs/yd³ lime, 3 lbs/yd³ starter fertilizer, and 0.5 lbs/yd³ wetting agent. The individual seeds were placed in flats of 32 plants each in an 8×4 configuration resulting in a 212 plants/m² density.

The plants were grown in a glass greenhouse with of a day length of 16 hours and growth conditions of daytime temperature at 22.22 °C, and nighttime temperature at 19.44 °C. Plants were imaged at first visual sight of the plumule using a Hyperspectral Visible and Near InfraRed camera and a short-wave infrared camera. The cameras capture reflectance between 400 nm and 2500 nm. Plants grown under normal conditions were imaged daily from 5 days after emergence (DAE) until approximately 14 DAE (V0 to V5 growth stage). Data were processed using proprietary software into average reflectance spectra of individual plants and physiologically relevant features. These data were standardized and processed using R. Custom algorithms were developed to adjust data and remove variability. Data were processed using a modified version of Mahalanobis distance to determine differences between treatments. Resulting distance matrices were log-transformed, with values over 3 considered significantly different.

### Results and Discussion:

The log transformation of Mahalanobis distance for soybeans treated with fluopyram (FLU+Polymer or FLU+FST+IST+Biological+Polymer) were significantly different compared to wild-type control for both varieties tested, with values ranging from 3.7 to 6.2 (Table 19). The FST+IST+Biological+Polymer treatment was significantly different compared to control only on P31A22X, while the FST+IST+Biological nematicide was significantly different compared to the control only on P72A21X (Table 19). In comparing treatment differences across both varieties, both treatments with fluopyram were significantly different compared to the FST+IST+Polymer and the FST+IST+Biological+Polymer treatments (Table 19). The FST+IST+Biological nematicide treatment was significantly different compared to the FST+IST+Polymer treatment for both P31A22X and P72A21X (Table 19).

For visible hyperspectral characteristics, the average pixel area trait had the most differences to contrast for all seed treatments among all plant ages tested for both P31A22X and P72A21X (Table 20). The green, yellow, and brown indices were different for treatments at different timepoints but did not have the magnitude of difference evident as compared to the total pixel area calculations (Table 20).

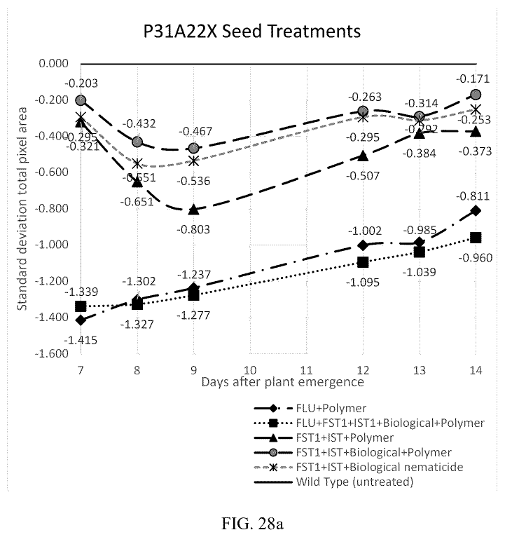

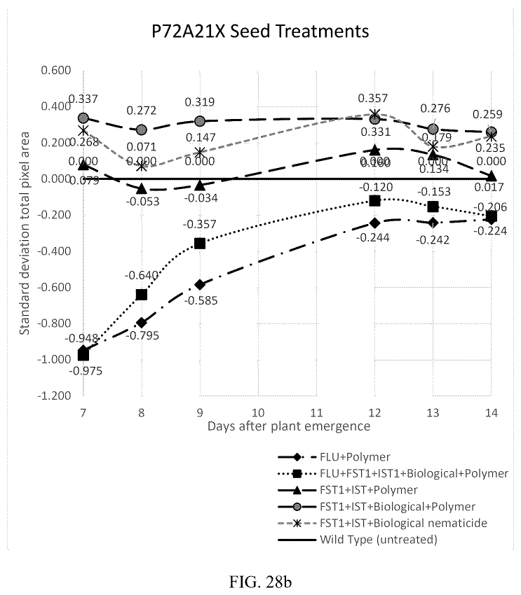

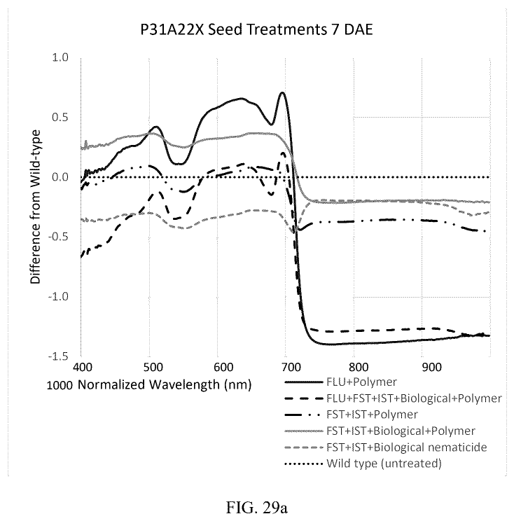

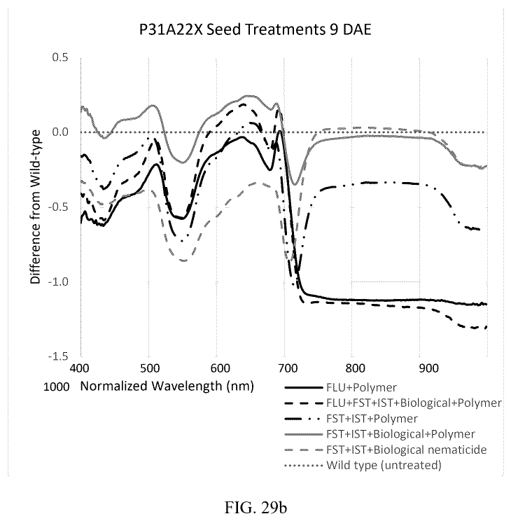

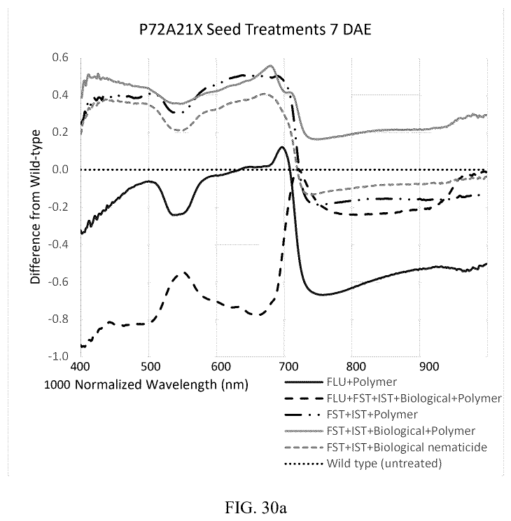

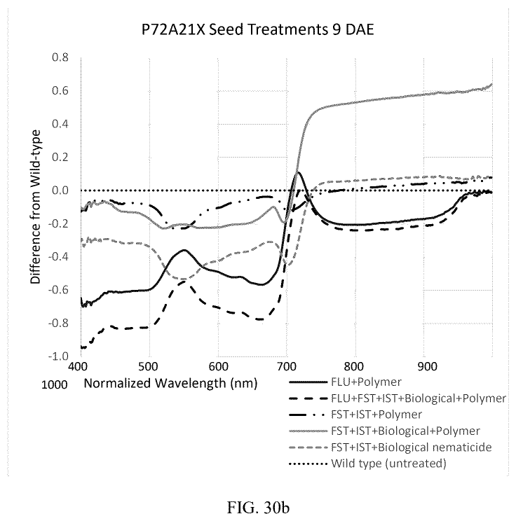

When the pixel area data were graphed, both fluopyram treatments had the lowest difference across all plant ages compared to the wild-type control and the other seed treatments for both P31A22X and P72A21X (FIG. 28). In general, the FST+IST+Biological+Polymer treatment had the best performance of all the treatments tested across all plant stages for both varieties (FIG. 28). The FST+IST+Biological nematicide treatment was the second-best performing treatment across all growth stages for both varieties (FIG. 28). Both the FST+IST+Biological+Polymer treatment and the FST+IST+Biological nematicide treatments had better overall performance at each plant stage for both varieties when compared to the FST+IST+Polymer treatment (FIG. 28). Different combinations of seed treatment mixtures can result in unique patterns in spectral reflectance, as detected by hyperspectral imaging. Observing the patterns for P31A22X, both seed treatments containing fluopyram have an identifiable dip in the mid-500 nm region at 7 DAE that becomes uniform across all treatments at the later age of 9 DAE (FIG. 29). At both plant ages, the behavior at wavelengths larger than 700 nm is divergent across treatments, with the seed treatments containing fluopyram diverging from the others (FIG. 29). For P72A21X, at 7 DAE, the seed treatments that do not contain fluopyram are similar in the visible region from 400-700 nm (FIG. 30). The FST+IST+Biological+Polymer retains higher reflectance compared to the other seed treatment mixtures (FIG. 30). The seed treatments containing fluopyram have opposite patterns at their mid-500 nm region which was not the case in P31A22X (FIG. 30). Likewise, the two seed treatments with fluopyram have different reflectance beyond the 700 nm wavelength. At 9 DAE, the main distinction is between the seed treatments containing fluopyram and the seed treatments that do not contain fluopyram (FIG. 30).

FST+IST+Biological+Polymer retains the highest reflectance beyond 700 nm, while the mid-500 nm region for the seed treatment containing fluopyram are moving in the same direction (FIG. 30).

The data indicate the potential to differentiate combinations of fungicide+insecticide+biological soybean seed treatment mixtures with or without a polymer using hyperspectral imaging on young plants from the V0 to V5 growth stage. This differentiation can be seen in morphological data (pixel area, green index, yellow index, brown index) and hyperspectral reflectance signature pattern at different timepoints.

TABLE 19
Combinations of fungicide + insecticide + biological components with
and without a polymer as seed treatment mixtures used in soybean hyperspectral experiments

| | | | | Rate (mg |
|---|---|---|---|---|
| n | Treatment | Component | Type | ai/seed) |
| 1 | Wild-type (not treated) | Not treated | | |
| 2 | FST + IST + Polymer | Oxathiapiprolin | Fungicide | 0.012 |
| | | Metalaxyl | Fungicide | 0.0065 |
| | | Penfluten | Fungicide | 0.0041 |
| | | Prothioconazole | Fungicide | 0.0082 |
| | | Imidacloprid | Insecticide | 0.075 |
| | | Polymer | Polymer | 0.04 |
| | | Green Color | Colorant | 0.02 |
| 3 | FST + IST + Biological + | Oxathiapiprolin | Fungicide | 0.012 |
| | Polymer | Metalaxyl | Fungicide | 0.0065 |
| | | Penfluten | Fungicide | 0.0041 |
| | | Prothioconazole | Fungicide | 0.0082 |
| | | Imidacloprid | Insecticide | 0.075 |
| | | Biological + Polymer | Biological + Polymer | |
| | | Green Color | Colorant | 0.02 |
| 4 | FST + IST + Biological | Oxathiapiprolin | Fungicide | 0.012 |
| | nematicide | Metalaxyl | Fungicide | 0.0065 |
| | | Penfluten | Fungicide | 0.0041 |
| | | Prothioconazole | Fungicide | 0.0082 |
| | | Imidacloprid | Insecticide | 0.075 |
| | | Biological nematicide | Biological nematicide | |
| | | Green Color | Colorant | 0.02 |
| 5 | FLU + FST + IST + Polymer | Fluopyram | Fungicide | 0.095 |
| | | Oxathiapiprolin | Fungicide | 0.012 |
| | | Metalaxyl | Fungicide | 0.0065 |
| | | Penfluten | Fungicide | 0.0041 |
| | | Prothioconazole | Fungicide | 0.0082 |
| | | Imidacloprid | Insecticide | 0.075 |
| | | Polymer | Polymer | 0.04 |
| | | Green Color | Colorant | 0.02 |
| 6 | FLU + FST + IST + Biological + | Fluopyram | Fungicide | 0.095 |
| | Polymer | Oxathiapiprolin | Fungicide | 0.012 |
| | | Metalaxyl | Fungicide | 0.0065 |
| | | Penfluten | Fungicide | 0.0041 |
| | | Prothioconazole | Fungicide | 0.0082 |
| | | Imidacloprid | Insecticide | 0.075 |
| | | Polymer | Polymer | 0.04 |
| | | Polymer | Polymer | 0.04 |
| | | Green Color | Colorant | 0.02 |

TABLE 20
Mahalanobis distance (log transformation of standard deviations) of soybean varieties P31A22X
or P72A21X treated with combinations of fungicide + insecticide + biological components
with and without a polymer as seed treatment mixtures and wild-type (WT, not treated seed)

| | | | | | | FLU + FST + | |
|---|---|---|---|---|---|---|---|
| | | | FST + - | FST + IST + | | IST + | FST + IST + |
| | | Wild-type | IST + | Biological + | FLU + | Biological + | Biological |
| Genotype | Seed Treatment A | (not treated) | Polymer | Polymer | Polymer | Polymer | nematicide |
| P31A22X | Wild-type (not treated) | 0 | | | | | |
| P31A22X | FST + IST + Polymer | 1.6 | 0 | | | | |
| P31A22X | FST + IST + Biological + Polymer | 3 | 2.0023 | 0 | | | |
| P31A22X | FLU¹ + Polymer | 3.7 | 3.1452 | 3.4078 | 0 | | |
| P31A22X | FLU + FST + IST + Biological + | 6.2 | 4.1335 | 3.182 | 1.9738 | 0 | |
| | Polymer | | | | | | |
| P31A22X | FST + IST + Biological nematicide | 2.4 | 1.3484 | 3.0305 | 3.2735 | 4.1143 | 0 |
| P72A21X | Wild-type (not treated) | 0 | | | | | |
| P72A21X | FST + IST + Polymer | 3.1 | 0 | | | | |
| P72A21X | FST + IST + Biological + Polymer | 2.7 | 2.2066 | 0 | | | |
| P72A21X | FLU + Polymer | 4.9 | 3.3023 | 3.102 | 0 | | |
| P72A21X | FLU + FST + IST + Biological + | 3.7 | 3.1575 | 3.1454 | 2.6094 | 0 | |
| | Polymer | | | | | | |
| P72A21X | FST + IST + Biological nematicide | 3.1 | 2.6164 | 3.0882 | 3.6123 | 3.1249 | 0 |

¹FLU = fluopyram, FST = fungicide seed treatment, IST = insecticide seed treatment

TABLE 21
Hyperspectral differences¹ of soybean varieties P31A22X or P48A60X treated with combinations of fungicide + insecticide +
biological components with and without a polymer as seed treatment mixtures compared to WT (wild-type not treated control).

| | | P31A22X | | | | P72A21X | | | |
|---|---|---|---|---|---|---|---|---|---|
| | | Avg | Avg | Avg | Avg | Avg | Avg | Avg | Avg |
| Plant | | Pixel | Green | Yellow | Brown | Pixel | Green | Yellow | Brown |
| age | Seed Treatment - WT Comparison | Area | Index | Index | Index | Area | Index | Index | Index |
| 7 | (FST + IST + Polymer) - WT | −0.321 | −1.147 | 1.425 | −1.093 | 0.079 | −0.509 | −0.665 | −0.740 |
| 7 | (FST + IST + Biological + Polymer) - WT | −0.203 | −0.829 | 1.342 | −1.064 | 0.337 | −0.618 | 0.703 | −1.034 |
| 7 | (FST + IST + Biological nematicide) - WT | −1.415 | −1.199 | −0.284 | −1.117 | −0.948 | −1.538 | −1.724 | −1.495 |
| 7 | (FLU + Polymer) - WT | −1.339 | −1.016 | −1.191 | −0.721 | −0.975 | −1.225 | 0.228 | −0.967 |
| 7 | (FLU + FST + IST + Biological + Polymer) - WT | −0.295 | −0.951 | 0.190 | −0.924 | 0.268 | −0.459 | 1.018 | −0.706 |
| 8 | (FST + IST + Polymer) - WT | −0.651 | 0.325 | 1.354 | −1.230 | −0.053 | 1.371 | −0.189 | −0.293 |
| 8 | (FST + IST + Biological + Polymer) - WT | −0.432 | 1.222 | 1.798 | −0.532 | 0.272 | −0.105 | −0.231 | 0.412 |
| 8 | (FST + IST + Biological nematicide) - WT | −1.302 | 0.341 | −0.030 | −0.413 | −0.795 | −0.944 | −0.857 | −0.604 |
| 8 | (FLU + FST + IST + Polymer) - WT | −1.327 | 1.571 | −0.712 | 1.051 | −0.640 | −0.578 | −0.026 | 0.097 |
| 8 | (FLU + FST + IST + Biological + Polymer) - WT | −0.551 | 1.118 | 0.727 | 0.914 | 0.071 | 0.899 | 2.071 | −2.218 |
| 9 | (FST + IST + Polymer) - WT | −0.803 | 0.612 | 0.213 | −1.230 | −0.034 | 0.964 | 0.005 | −0.392 |
| 9 | (FST + IST + Biological + Polymer) - WT | −0.467 | 1.521 | 0.872 | −0.379 | 0.319 | −0.424 | −0.347 | −0.960 |
| 9 | (FST + IST + Biological nematicide) - WT | −1.237 | −0.291 | −1.376 | 0.121 | −0.585 | −0.224 | −1.160 | 0.155 |
| 9 | (FLU + FST + IST + Polymer) - WT | −1.277 | 1.137 | −0.956 | −0.126 | −0.357 | 0.101 | −1.353 | 0.578 |
| 9 | (FLU + FST + IST + Biological + Polymer) - WT | −0.536 | 0.717 | 0.281 | 0.940 | 0.147 | 1.218 | 0.888 | −1.879 |
| 12 | (FST + IST + Polymer) - WT | −0.507 | 0.830 | −0.811 | 0.717 | 0.160 | 1.301 | −0.646 | 0.764 |
| 12 | (FST + IST + Biological + Polymer) - WT | −0.263 | 1.438 | −0.742 | 1.217 | 0.331 | 1.492 | −0.813 | 0.388 |
| 12 | (FST + IST + Biological nematicide) - WT | −1.002 | −0.959 | −1.838 | 1.510 | −0.244 | −0.070 | −1.740 | 1.865 |
| 12 | (FLU + FST + IST + Polymer) - WT | −1.095 | −0.962 | −1.100 | 1.340 | −0.120 | 0.029 | −1.064 | 1.127 |
| 12 | (FLU + FST + IST + Biological + Polymer) - WT | −0.295 | 1.046 | −0.594 | 0.779 | 0.357 | 0.822 | −0.230 | −0.227 |
| 13 | (FST + IST + Polymer) - WT | −0.384 | 0.363 | −0.794 | 0.705 | 0.134 | 0.264 | −0.945 | 0.598 |
| 13 | (FST + IST + Biological + Polymer) - WT | −0.292 | 0.838 | −0.693 | 0.992 | 0.276 | 1.582 | −0.592 | −0.007 |
| 13 | (FST + IST + Biological nematicide) - WT | −0.985 | −1.759 | −1.898 | 1.430 | −0.242 | −0.678 | −1.710 | 2.008 |
| 13 | (FLU + FST + IST + Polymer) - WT | −1.039 | −1.199 | −1.157 | 1.653 | −0.153 | −0.668 | −1.123 | 1.027 |
| 13 | (FLU + FST + IST + Biological + Polymer) - WT | −0.314 | 0.738 | −0.456 | 0.538 | 0.179 | 0.438 | −0.201 | −0.712 |
| 14 | (FST + IST + Polymer) - WT | −0.373 | −1.429 | −0.681 | 0.468 | 0.017 | 0.579 | −0.709 | 0.252 |
| 14 | (FST + IST + Biological + Polymer) - WT | −0.171 | −0.014 | −0.661 | 0.776 | 0.259 | 1.811 | −0.698 | 0.126 |
| 14 | (FST + IST + Biological nematicide) - WT | −0.811 | −1.107 | −1.878 | 1.789 | −0.224 | −1.356 | −1.923 | 1.948 |
| 14 | (FLU + FST + IST + Polymer) - WT | −0.960 | −1.339 | −1.188 | 1.480 | −0.206 | −0.696 | −0.916 | 0.712 |
| 14 | (FLU + FST + IST + Biological + Polymer) - WT | −0.253 | 0.467 | −0.440 | 0.460 | 0.235 | 0.498 | 0.107 | −0.828 |

FLU = fluopyram, FST = fungicide seed treatment, IST = insecticide seed treatment
¹Positive values indicate better performance compared to WT (wild-type not treated control), negative values indicate worse performance compared to WT.

1. A computer-implemented high throughput total reflectance spectral method for evaluating efficacy of a seed treatment protocol in a greenhouse comprising:
a) subjecting a plurality of seeds to a seed treatment protocol;
b) allowing the plurality of seeds to germinate into plants;
c) measuring plant response to the seed treatment protocol at a pre-determined growth stage using a high throughput total reflectance spectral analysis method encompassing the wavelength range from 250 nm to 2500 nm and using reflectance spectra interpolation of discrete wavelength measurements between 1.0 to 4.0 nm; and
d) determining the efficacy of the seed treatment protocol based upon the plant response in step c).

2. The computer-implemented high throughput total reflectance spectral analysis method of claim 1, wherein step c) comprising:
c1) measuring the plant response at a wave length range of 400-1000 nm at 1.0-2.0 nm increments, preferably at 1.25-1.75 nm increments, most preferably at 1.50 nm increments; and
c2) measuring the plant response at a wave length range of 900-2500 nm at 2.0-4.0 nm increments, preferably at 2.5-4.0 nm increments, most preferably at 3.75 nm increments.

3. The computer-implemented high throughput total spectral analysis method of any of the preceding claims, wherein the high throughput total spectral analysis method is hyperspectral image data analysis.

4. The computer-implemented high throughput total spectral analysis method of any of the preceding claims, wherein the seed treatment protocol is selected from the group consisting of biological and/or chemical a) amendments b) biologicals c) biostimulants, d) fungicides e) insecticides f) nematicides g) plant growth promoters h) plant growth stimulants and/or combinations thereof.

5. The computer-implemented high throughput total spectral analysis methods of claim 4, wherein,
a) the fungicide is selected from the group consisting of azoxystrobin, difenconazole, ethaboxam, fludioxonil, fluopyram, fluxapyroxad, fluoxastrobin, ipconazole, inpyrfluxam, myclobutanil, oxathiapiprolin, penfluten, picoxystrobin, prothioconazole, pyraclostrobin, mefenoxam, metalaxyl, sedaxane, tebuconazole, thiabendazole, triconazole, zoxamide or mixtures thereof;
b) the insecticide is selected from the group consisting of brofanilide, chlorantraniliprole, clothianidin, cyantraniliprole, imidacloprid, spinosad, spinetoram, sulfoxaflor, tefluthrin, thiamethoxam, triflumexopyrim or mixtures thereof;
c) the nematicide is selected from the group consisting of abamectin, fluopyram, thiodicarb, and tioxazafin, or biologicals with nematicide properties selected from Bacillus species or Burkholderia species;
d) the amendment, biological or plant stimulant component is selected from Acinetobacter species, Arthrobacter species. Bacillus species, Bradyrhizobium species, Buricholderia species, Burkholderia species, Carnobaterium species, Flavobacterium species, Kosakonia species, Methyl bacterium species, Pantoea species, Pasteuria species, Penicillium species, Pseudomonas species, Rahnella species, Rhizobium species, Sphingobium species, Stenotrophomonas species, Trichoderma species, or Variovorax species.

6. The computer-implemented high throughput total spectral analysis method of claim 1, wherein in step c) plants are evaluated at time intervals between the V0 to V6 growth stage.

7. The computer-implemented high throughput total spectral analysis method of any of the preceding claims, wherein images are captured by a hyperspectral camera and data analysis system, analyzed by statistical software and converted into the plant response, wherein the plant response is measured by using at least one objective measurement that correlates with plant phenotype, increased or decreased plant size, plant growth, plant health and/or plant vigor, wherein the at least one objective measurement is selected from the group consisting of total pixel area, green pixel area, percent green pixel, yellow pixel area, percent yellow pixel, brown pixel area, percent brown pixel, green index, yellow index, brown index, spectral signature, spectral reflectance, vegetative index, Normalized Difference Vegetation Index (NDVI), Green biomass Density (GBD), Green Leaf Area Index (GLAI), Absorbed Photosynthetically-Active Radiation (APAR), Gross Primary Production (GPP), and water slope.

8. The computer-implemented high throughput total spectral analysis method of any of the preceding claims further comprising using a seed not treated with a seed treatment protocol as a control, and measuring the difference between the plant response of a plant grown from a treated seed (X) and a plant grown from an not treated seed (Y), and calculating the difference between X and Y in terms of a statistical measurement, wherein the statistical measurement is selected from the group consisting of Mahalanobis distance, Bhattacharya distance, the Wasserstein metric, and Kolmogorov-Smirnov test and wherein resulting value of each treatment is more than or equal to a determined significance threshold.

9. The computer-implemented high throughput total spectral analysis method of any of the preceding claims further comprising treating a first seed with a commercial seed treatment protocol T1 and treating a second seed with seed treatment protocol T2, and measuring the difference between the plant response of a plant grown from the second seed treated with seed treatment T2 (A) and a plant grown from the first seed treated with a commercial seed treatment T1 (B), and calculating the difference between A and B in terms of a statistical measurement, wherein the statistical measurement is selected from the group consisting of Mahalanobis distance, Bhattacharya distance, the Wasserstein metric, or Kolmogorov-Smirnov test and wherein the resulting value of each treatment is more than or equal to a determined significance threshold.

10. The computer-implemented high throughput total spectral analysis method of any of the preceding claims, wherein the plurality of seeds is selected from the group of crops selected from the group consisting of alfalfa, barley, canola, cotton, maize, pulse crops, soybean, sorghum sunflower, wheat, and vegetables.

11. The computer-implemented high throughput total spectral analysis method of any of the preceding claims further comprising making a determination regarding the seed treatment protocol, wherein the determination is one or more of:
i) approval,
ii) comparison,
iii) rejection,
iv) amendment,
v) contrast,
vi) combination,
vii) differentiation,
viii) prediction of an outcome, or
ix) quantification of an outcome,
of the seed treatment protocol.

12. Use of the computer-implemented high throughput total spectral analysis method of claims 1-11 for rapid screening of one or more seed treatment protocols.

13. Method for obtaining data related to a plant response of a plant grown from a seed treated with one or more amendment, biological, biostimulant, fungicide, insecticide, or nematicide seed treatments and/or combinations of different seed treatments, different rates of amendment, biological, biostimulant, fungicide, insecticide and/or nematicide active components using the computer-implemented total spectral analysis methods in any of the preceding claims.

14. The method of claim 14, wherein the high throughput total spectral analysis method comprises obtaining the data using hyperspectral imaging data analysis of seedlings grown from a treated seed in a greenhouse setting at any time from V0 to V6 growth stages.

15. The method of claims 14-15, further comprising using the obtained data to;
a) differentiate a seed treatment comprising one or more of amendment, biostimulant, biological, fungicide, insecticide, and/or nematicide seed treatment including any treatment amendments combination of seed treatment actives at different rates, use of different formulations, adjuvants, formulation auxiliaries;
b) quantification the plant response of a plant grown from a seed treated with one or more amendment, biostimulant, biological, fungicide, insecticide, and/or nematicide seed treatments and/or combinations of different seed treatments, different rates of amendment, biostimulant, biological, fungicide, insecticide, and/or nematicide active components;
c) contrast a seed treatment comprising one or more of amendment, biostimulant, biological, fungicide, insecticide, and/or nematicide seed treatment including any treatment amendments, combination of seed treatment actives at different rates, and use of different formulations, adjuvants, and formulation auxiliaries;
d) use any of a), b) and/or c) as a screening tool for testing, advancing or discarding a seed treatment comprising one or more of amendment, biostimulant, biological, fungicide, insecticide, and/or nematicide seed treatment including any treatment amendments, combination of seed treatment actives at different rates, and use of different formulations, adjuvants, formulation auxiliaries.

16. A computer-implemented method for evaluating an efficacy of a seed treatment comprising:
a) receiving hyperspectral image and total reflectance data of a plant grown from a seed treated with one or more of a seed treatment protocol using hyperspectral imaging analysis method encompassing wavelength range of 400-2500 nm and using reflectance spectra interpolation of discrete wavelength measurements between 1.0 to 4.0 nm; and
b) determining plant response to the seed treatment protocol using hyperspectral image and total reflectance data analysis.

17. A computer-implemented method for obtaining data related to a plant response of a plant grown from a seed treated with one or more amendment, biostimulant, biological, fungicide, insecticide, and/or nematicide seed treatments and/or combinations of different seed treatments, different rates of amendment, biostimulant, biological, fungicide, insecticide, and/or nematicide active components using hyperspectral imaging analysis method, the method comprising:
a) receiving total reflectance of a plant from a seed treated with one or more of a seed treatment protocol using a hyperspectral camera and imaging system encompassing the wavelength range from 400 nm to 2500 nm and using reflectance spectra interpolation of discrete wavelength measurements between 1.0 to 4.0 nm; and
b) processing the reflectance data with data analysis statistical software to correlate total reflectance with one or more of plant phenotype, plant growth, plant health and/or vigor.

18. The method of claim 17, further comprising using the obtained data to;
a) differentiate a seed treatment comprising one or more of amendment, biostimulant, biological, fungicide, insecticide, and/or nematicide seed treatment including any treatment combination of seed treatment actives at different rates, use of different formulations, adjuvants, formulation auxiliaries;
b) quantification the plant response of a plant grown from a seed treated with one or more amendment, biostimulant, biological, fungicide, insecticide, and/or nematicide seed treatment and/or combinations of different seed treatments, different rates of amendment, biostimulant, biological, fungicide, insecticide, and/or nematicide active components;
c) contrast a seed treatment comprising one or more of amendment, biostimulant, biological, fungicide, insecticide, and/or nematicide seed treatment seed treatment including any treatment combination of seed treatment actives at different rates, and use of different formulations, adjuvants, formulation auxiliaries; or
d) use any of a), b) and/or c) as a screening tool for testing, advancing or discarding a seed treatment comprising one or more of amendment, biostimulant, biological, fungicide, insecticide, and/or nematicide seed treatment including any treatment combination of seed treatment actives at different rates, and use of different formulations, adjuvants, formulation auxiliaries.

19. A system for evaluating an efficacy of a seed treatment comprising;
one or more processors, one or more memory units, one or more non-transitory computer-readable storage media storing one or more computer instructions which, when executed by the one or more processors, cause the one or more processors to perform:
processing of total reflectance using a hyperspectral camera and imaging platform encompassing the wavelength range from 400 nm to 2500 nm and using reflectance spectra interpolation of discrete wavelength measurements between 1.0 to 4.0 nm to obtain data that correlates with one or more of plant phenotype, plant growth, plant health and/or vigor of a plant grown from a seed treated with one or more of amendment, biostimulant, biological, fungicide, insecticide, and/or nematicide seed treatment and/or combinations of different seed treatments, different rates of amendment, biostimulant, biological, fungicide, insecticide, and/or nematicide active components.
